(19) United States
(12) Patent Application Publication
(10) Pub. No.: US 2009/0180989 A1
(43) Pub. Date: Jul. 16, 2009

(54) Compositions and methods for delivering nucleotide sequences to vertebrates

(75) Inventor: Alex J. Harvey, Athens, GA (US)

Correspondence Address: Synageva BioPharma Corp., 111 RIVERBEND ROAD, ATHENS, GA, 30605, US

(73) Assignee: Synageva BioPharma Corp.

(21) Appl. No.: 12/380,475

(22) Filed: Feb. 27, 2009

Related U.S. Application Data
(63) Continuation-in-part of application No. 11/542,093, filed on Oct. 3, 2006, now Pat. No. 7,524,626.
(60) Provisional application No. 60/723,659, filed on Oct. 5, 2005.
(60) Provisional application No. 61/197,555, filed on Oct. 28, 2008.

(51) Int. Cl. A61K 48/00; C12N 15/867; C12P 21/00; A61K 35/76

(52) U.S. Cl. 424/93.2; 435/456; 800/4

(57) ABSTRACT

The invention includes methods of producing viral particles which include introducing into avian cells a nucleotide sequence encoding a replication deficient retroviral vector and introducing into the avian cells nucleotide sequence encoding products required for replication of the replication deficient retroviral vector, harvesting the viral particles and can include administering the viral particles to vertebrate cells.

### RELATED APPLICATION INFORMATION

This application claims the benefit of U.S. provisional application No. 61/197,555, filed Oct. 28, 2008, the disclosure of which is incorporated in its entirety herein by reference, and is a continuation-in-part of U.S. patent application Ser. No. 11/542,093, filed Oct. 3, 2006, the disclosure of which is incorporated in its entirety herein by reference, which claims the benefit of U.S. provisional application No. 60/723,659, filed Oct. 5, 2005, the disclosure of which is incorporated in its entirety herein by reference.

### BACKGROUND

This invention is directed to the production of viral particles from retroviruses which are capable of transducing cells, for example, avian cells, including germ cells. In particular, replication deficient retroviral vector particles can be produced in accordance with the invention.

Replication deficient retroviruses are particularly useful in recombinant methodologies such as gene therapy procedures and in the production of transgenic animals, for example, transgenic avians. One particularly useful transgenic animal that can be produced using replication deficient retroviruses is the transgenic chicken.

The production of an avian egg begins with formation of a large yolk in the ovary of the hen with the unfertilized ovum formed on the yolk sac. After ovulation, the yolk and ovum pass into the infundibulum of the oviduct where it is fertilized, if sperm are present, and then moves into the magnum of the oviduct which is lined with tubular gland cells. These cells secrete the egg-white proteins, including ovalbumin, ovomucoid, ovoinhibitor, conalbumin, ovomucin and lysozyme, into the lumen of the magnum where they are deposited onto the avian embryo and yolk. Researchers have been successful in producing transgenic avians in which the tubular gland cells produce the exogenous protein and secrete it into the oviduct lumen along with the egg white protein for packaging into an egg. See, for example, Harvey et al, Nature Biotechnology (2002) vol 20, p 396-399, the disclosure of which is incorporated in its entirety herein by reference and U.S. Pat. No. 6,730,822, issued May 4, 2004, the disclosure of which is incorporated in its entirety herein by reference. This system offers outstanding potential as a protein bioreactor because of the high levels of protein production, proper folding and useful post-translation modifications of the target protein, the ease of product recovery, and the shorter developmental period of chickens compared to other animal species used for heterologous gene expression. Significantly, retrovrial production in transgenic animals such as chickens can be limited by the size of the insert allowed by the retrovirus. For example, inserts contained in the retroviruses can be limited to 2 to 3 kb. Production of integration competent virus is inhibited when insert size constraints are exceeded. Important methods used to produce transgenic avians such as chickens using retroviruses involve the introduction of replication deficient yet integration competent retroviral particles into embryonic cells.

Replication deficient retroviral vectors lack certain genes required for successful reproduction of the virus. Traditionally, to produce replication deficient retroviral vectors, nucleotide sequences encoding replication deficient retroviruses have been transfected into cells which stably produce the gene products required for replication of the replication deficient retrovirus. That is, certain nucleotide sequences required for the replication of the retrovirus are missing from the retrovirus but are present in the genome of the cell in which the viral particles are produced. One system that has been used to produce replication deficient ALV retroviruses involves the use of Senta cells and Isolde cells (Cosset et al (1993) Virology vol 195, p 385-395). The process involves first transfecting nucleotide sequences encoding the replication deficient retrovirus into the Senta cells which stably produce the gag, pol and envE proteins. Viral titer obtained in the Senta cells is typically <1000/ml. To increase the titer, the viral particles produced in the Senta cells are used to transduce Isolde cells which stably produce the gag, pol and envA proteins. The retrovirus produced in this manner can contain a neomycin resistance gene which allows for selection of Isolde clones or single colonies, some of which will produce particles at high titers >10,000/ml. In spite of the production of useable amount of viral particles being produced, the titers are still relatively low using this procedure. In addition, the process is laborious and time consuming, taking typically about three months.

What is needed are new methods of producing viral particles which require less time and less labor and allow for the insertion of larger nucleotide sequences in the recipient genome and result in high titers.

### SUMMARY

A retrovirus production system has been developed and is described herein in which replication deficient retroviral particles can be produced using a minimal amount of labor, can be produced in as little as 2 days, can yield titers typically ten fold or more greater than that obtained by conventional methods, can provide for a substantial increase in the size of nucleotide insert that can be introduced into the retroviral vector by deletion of nucleotide sequence from the retroviral vector including as many as three major structural genes, for example, gag (typically about 2000 nucleotides), pol (typically about 2300 nucleotides) and/or env (typically about 1500 nucleotides) protein genes. In one embodiment, a nucleotide sequence encoding a replication deficient retrovirus or retroviral vector is introduced into a cell such as a fibroblast cell along with nucleotide sequence that provides for replication of the replication deficient retrovirus or retroviral vector, in particular, nucleotide sequences encoding two or more of the gag, pol and env proteins are introduced into the cell. In one particularly useful embodiment, nucleotide sequences encoding all three of the gag, pol and env proteins are required for replication of the replication deficient viral vector and are introduced into the cell.

In one embodiment, methods of the invention include introducing, for example, transfecting (e.g., a transient transfection) into a cell a nucleotide sequence encoding a retroviral vector wherein the retroviral vector is replication deficient (e.g., a single nucleotide sequence containing a polynucleotide encoding a replication deficient retrovirus); introducing, for example, transfecting into the cell nucleotide sequences that are transcriptionally and/or translationally functional in the cell wherein the nucleotide sequences encode products required for replication of the replication deficient virus such as nucleotide sequences which encode gag, pol and/or env proteins; and harvesting viral particles.

In one particularly useful embodiment of the invention, each nucleotide sequence introduced into the cell (i.e., nucleotide sequence(s) encoding the replication deficient retroviral vector and nucleotide sequence(s) encoding products required for replication of the replication deficient virus) is introduced in a transient manner. That is the nucleotide sequences are not expected to replicate in the cell and are not expected to integrate in the cellular genome. For example, the nucleotides sequences can be introduced in the cell contained in one or more bacterial plasmid vectors. The invention also contemplates, the nucleotide sequence(s) encoding products required for replication of the replication deficient virus being introduced into the cell in a transient manner and the nucleotide sequence(s) encoding the retroviral vector being introduced into the cell in a manner which provides for stable integration of the nucleotide sequence(s) into the genome of the cell. Methods are well known in the art that provide for stable integration of desired nucleotide sequences in the genome of cells, for example, cells of cell lines. For example, it is known in the art that replication deficient retroviral vectors can be stably integrated in a cellular genome.

The nucleotide sequence(s) encoding products required for replication of the replication deficient virus may be introduced into the cell before introduction of the nucleotide sequence(s) encoding the retroviral vector; the nucleotide sequence(s) encoding products required for replication of the replication deficient virus may be introduced into the cell at about the same time as the introduction of the nucleotide sequence(s) encoding the retroviral vector; or the nucleotide sequence(s) encoding products required for replication of the replication deficient virus may be introduced into the cell after introduction of the nucleotide sequence(s) encoding the retroviral vector.

In one embodiment, nucleotide sequences that encode products that provide for replication of the replication deficient retroviral vector are contained in one or more plasmids, for example, one plasmid for each nucleotide sequence. In certain useful embodiments, the replication deficient retroviral vector is contained in a plasmid. When nucleotide sequences are contained in a plasmid in accordance with the invention, those sequences will typically be introduced transiently into the cell.

Certain cells and cell lines that can be very useful in the present invention are avian cells (e.g., avian fibroblast cells) and avian cell lines (e.g., avian fibroblast cell lines) obtained from avians such as, chicken, turkey, duck, goose, quail, pheasants, parrots, finches, hawks, crows and ratites including ostrich, emu and cassowary. In one particularly useful embodiment, a chicken fibroblast cell line is used. However, the invention is not limited to the use of fibroblast cells and specifically contemplates any useful cell lines such as mouse cell lines, human cell lines, hamster cell lines such as CHO cells and chicken cell lines such as LMH, LMH2a cells.

In one particularly useful embodiment, the nucleotide sequence (e.g., DNA, RNA) encoding a replication deficient retroviral vector encodes a retroviral vector based upon an avian retrovirus. Examples of avian retroviruses include, without limitation, Avian Leukemia/Leukosis Viruses (ALV), for example, and without limitation, RAV-0, RAV-1, RAV-2; Avian Sarcoma Viruses (ASV); Avian Sarcoma/Acute Leukemia Viruses (ASLV) including, without limitation, Rous Sarcoma Virus (RSV); Fujinami Sarcoma Viruses (FSV); Avian Myeloblastosis Viruses (AMV); Avian Erythroblastosis Viruses (AEV); Avian Myelocytomatosis Viruses (MCV), for example, and without limitation, MC29; Reticuloendotheliosis Viruses (REV), for example, and without limitation, Spleen Necrosis Virus (SNV). The invention also contemplates that the nucleotide sequence encoding a replication deficient retroviral vector can encode any useful retroviral vector, including, without limitation, retroviral vectors based upon Murine Leukemia Viruses (MLV); Molony Murine Sarcoma Viruses (MMSV); Moloney Murine Leukemia Viruses (MMLV); and lentiviruses (e.g., human immunodeficiency virus (HIV), feline immunodeficiency virus (FIV), bovine immunodeficiency virus (BIV) and simian immunodeficiency virus (SIV).

In one particularly useful embodiment, the nucleotide sequence(s) (e.g., DNA, RNA) that encodes the products required for replication of the replication deficient retrovirus is nucleotide sequence obtained or derived from the genome of an avian retrovirus. Examples of avian retroviruses which can provide such nucleotide sequences include, without limitation, Avian Leukemia/Leukosis Viruses (ALV), for example, and without limitation, RAV-0, RAV-1, RAV-2; Avian Sarcoma Viruses (ASV); Avian Sarcoma/Acute Leukemia Viruses (ASLV) including, without limitation, Rous Sarcoma Virus (RSV); Fujinami Sarcoma Viruses (FSV); Avian Myeloblastosis Viruses (AMV); Avian Erythroblastosis Viruses (AEV); Avian Myelocytomatosis Viruses (MCV), for example, and without limitation, MC29; Reticuloendotheliosis Viruses (REV), for example, and without limitation, Spleen Necrosis Virus (SNV). The invention also contemplates the nucleotide sequence encoding a product required for replication of the replication deficient virus being nucleotide sequence obtained or derived from the genome of any useful retrovirus, including, without limitation, Murine Leukemia Viruses (MLV); Molony Murine Sarcoma Viruses (MMSV); Moloney Murine Leukemia Viruses (MMLV); and lentiviruses (e.g., human immunodeficiency virus (HIV), feline immunodeficiency virus (FIV), bovine immunodeficiency virus (BIV) and simian immunodeficiency virus (SIV).

Included in one specific aspect of the invention are methods of producing a viral particle which comprise introducing (e.g., transfecting) into a fibroblast cell line nucleotide sequences required for replication of the replication defective retroviral vector, for example, nucleotide sequences encoding gag, pol and env proteins wherein the gag, pol and env protein coding sequences are under the control of a promoter that is functional in the fibroblast cell line; introducing (e.g., transfecting) into the fibroblast cell line a nucleotide sequence encoding a replication deficient retroviral vector; and harvesting the viral particles.

In one embodiment, the gag, pol and env protein coding sequences required for replication of the replication defective retroviral vector are contained in one or more plasmids. For example, the gag, pol and env protein coding sequences may all be contained in one plasmid or each may be contained in a separate plasmid. In another example, two of the gag, pol and env protein coding sequences (e.g., gag and pol) may be present on one plasmid and the third may be present on another plasmid (e.g., the env).

In one aspect, the nucleotide sequence encoding the retroviral vector is a provirus. That is, the nucleotide sequence encoding the retroviral vector is DNA that has been integrated into a host cell genome. In one embodiment, the nucleotide sequence encoding the retroviral vector is present in a plasmid.

In one particularly useful embodiment, the gag, pol and env protein encoding nucleotide sequences are from an avian retrovirus. Examples of avian retroviruses from which the gag, pol and env protein encoding nucleotide sequences may be obtained include, without limitation, Avian Leukemia/Leukosis Viruses (ALV), for example, and without limitation, RAV-0, RAV-1, RAV-2; Avian Sarcoma Viruses (ASV); Avian Sarcoma/Acute Leukemia Viruses (ASLV) including, without limitation, Rous Sarcoma Virus (RSV); Fujinami Sarcoma Viruses (FSV); Avian Myeloblastosis Viruses (AMV); Avian Erythroblastosis Viruses (AEV); Avian Myelocytomatosis Viruses (MCV), for example, and without limitation, MC29; Reticuloendotheliosis Viruses (REV), for example, and without limitation, Spleen Necrosis Virus (SNV). It is also contemplated that the gag, pol and env protein encoding nucleotide sequences required for replication of the replication defective retroviral vector can be derived or obtained from any useful retroviral vector, including, without limitation, retroviral vectors based upon Murine Leukemia Viruses (MLV); Molony Murine Sarcoma Viruses (MMSV); Moloney Murine Leukemia Viruses (MMLV); and lentiviruses (e.g., human immunodeficiency virus (HIV), feline immunodeficiency virus (FIV), bovine immunodeficiency virus (BIV) and simian immunodeficiency virus (SIV).

In certain embodiments, the nucleotide sequences required for replication of the replication defective retroviral vector may not all be from the same virus. For example, a gag protein may be from the Avian Leukosis Virus (ALV), a pol protein may be from the Molony Murine Sarcoma Virus (MMSV), and an env protein may be from the Avian Erythroblastosis Viruses (AEV). In another example, a gag protein may be from the Molony Murine Sarcoma Virus (MMSV) an env protein may be from the Avian Leukosis Virus (ALV). These are only examples provided for illustrative purposes and the invention is not limited thereto.

Though specific embodiments of the invention require three nucleotide sequences for replication of the replication defective retroviral vector, for example, sequences encoding invention the gag, pol and env proteins, the invention is not limited thereto. For example, only one or two nucleotide sequence may be required to provide products necessary for replication of the replication defective retroviral vector.

In one aspect, the invention is directed to methods of producing transgenic avians. The methods typically include harvesting viral particles produced as disclosed herein and introducing the harvested retroviral particles into avian embryo cells such as early stage embryos, for example, stage I to stage XII embryos, and thereafter obtaining a hatched chick derived from the embryo cells.

Protein produced in transgenic avians of the invention, for example, exogenous protein deposited in eggs laid by the transgenic bird (e.g., deposited in the egg white) can be isolated by employing standard purification methods well known in the art.

The invention also relates to the delivery of one or more therapeutic polypeptides to a vertebrate (e.g., avian, mammal, amphibian, reptile, human) host by administration of a replication-deficient, self-inactivating avian leucosis virus (ALV) transgenesis vector which may be produced in accordance with the invention.

In one embodiment, the invention is directed to a method of transiently introducing into a cell (e.g., an avian cell) a replication deficient retroviral vector (e.g., an avian retroviral vector) which contains a nucleotide sequence encoding a therapeutic protein. The vector is transiently introduced as are nucleotide sequences which encode products required for replication of the replication deficient retroviral vector (e.g., gag, pol and env proteins).

Typically, the invention includes harvesting the viral particles. Harvesting in this context can mean at least partially separating the viral particles from the cells in which the viral particles were produced. Harvesting can also include removing at least some of the medium (e.g., concentrating the viral particles). Virus particles can be produced in any suitable cell, including but not limited to one or a combination of the following: a fibroblast cell; an avian cell; a chicken cell; a DF-1 cell.

The method may include introducing the replication deficient retroviral vectors contained in the viral particles into the vertebrate cells by, for example, and without limitation, exposing the viral particles to vertebrate cells.

In one embodiment, the replication deficient retroviral vectors contain a coding sequence for a heterologous protein, for example, and without limitation, a therapeutic protein. Examples of such therapeutic proteins include human proteins, immunoglobulins, enzymes, fusion proteins, cytokines and others such as those disclosed herein.

The methods typically include introducing into the cell in which the viral particles are produced one or more nucleotide sequence under the control of a promoter that is functional in the cell wherein the nucleotide sequence(s) encode one or more products required for replication of the replication deficient retroviral vector (e.g., two or three of gag, pol and env).

In one embodiment where a transgenic avian such as a transgenic chicken is to be obtained in accordance with the invention, the harvested particles are introduced into avian blastodermal cells, for example, avian blastodemal cells which may be contained in a fertilized hard shell egg (e.g., a stage VI to stage XII egg).

In one embodiment, a transgenic avian (e.g., chicken, turkey, quail) is obtained which develops from the blastodermal cells which produces a heterologous protein (e.g., therapeutic protein) encoded by the coding sequence. The invention also contemplates the heterologous protein being deposited in eggs laid by such transgenic avian and further can include isolating the protein from the egg using standard techniques well know in the art.

In one embodiment, the invention is directed to administering to a vertebrate cell in vivo a replication deficient retroviral vector comprising a nucleotide sequence encoding a therapeutic polypeptide, wherein the avian retroviral vector is produced in DF-1 cells. In one embodiment, the retroviral vector integrates into the genome of a vertebrate target cell and expresses a nucleotide sequence encoding a therapeutic protein.

In methods of the invention it is contemplated that the retroviral vector of the viral particle made in accordance with the invention can be introduced into any vertebrate where such introduction would be useful, including, but not limited to avians (e.g., chickens). The introduction into such vertebrate cells can be accomplished by any useful method such as, without limitation, exposing the virus particles made in accordance with the invention to the vertebrate cells. In one embodiment, the vertebrate cells are transduced with the viral particles. In one embodiment, the vertebrate cells into which the retroviral vector is introduced are embryonic cells. In another example, the vertebrate cells into which the retroviral vector is introduced are somatic cells.

In one embodiment, the retroviral vector has a 5′ LTR and a 3′ LTR at least one of which is transcriptionally inactive upon integration into the vertebrate target cell. In one embodiment, the retrovirus contains a coding sequence for an exogenous protein (e.g., therapeutic protein) operably linked to a promoter. Examples of useful promoter types include promoters which are one or more of the following: constitutive promoter; tissue specific promoter; inducible promoter.

In one embodiment, the invention includes improved ALV-based expression vectors. For example, NLB was modified such that the LTRs would be self-inactivating or SIN. Of the 3′ LTR, 273 bp was deleted, which includes the enhancer and CAAT box of the U3 region (FIG. 4D). Because the U3 region at the 3′ end of the retroviral sequence serves as a template for a new U3 region present at the 5′ end of an integrated provirus, the inactivated 3′LTR is essentially copied to the 5′ LTR, thus inactivating the 5′ LTR. The neomycin resistance (neo) gene of pNLB, which serves as a means to titer retroviral particle preparations, is driven by the 5′ LTR promoter (FIG. 4A). Inactivation of the 5′ LTR would render the neo gene inactive, therefore, it was removed providing additional space for insert nucleic acid. Accordingly, the invention includes the use of retroviral vector that do not include a selectable cassette.

The new vector, termed pALV-SIN for ALV self-inactivation vector, is shown in FIG. 4C. Downstream of the 5′ LTR is the original packaging signal (ψ), partial gag and env coding sequences and the ALV constitutive transport element (CTE), all of which were part of pNLB. The CTE mediates the export of unspliced RNAs to the cytoplasm, thus facilitating the packaging of intact retroviral RNA (Yang and Cullen (1999) RNA 5(12): 1645-55. Any of the restriction sites between BamHI and NruI as shown in FIG. 4C could be used for insertion of DNA fragments without affecting the titer of the vector. Use of BpuAI, which resides 3′ of W, to clone in transgenes yielded which were not able to efficiently transduce chicken embryonic cells, suggesting that sequences between BpuAI and the 5′ BamHI site are required for function of the vector.

Certain references which may assist in applying the present invention, the disclosures of which are incorporated herein in their entirety by reference, include: Burns, J. C., T. Friedmann, et al. (1993). “Vesicular stomatitis virus G glycoprotein pseudotyped retroviral vectors: concentration to very high titer and efficient gene transfer into mammalian and nonmammalian cell” Proc Natl Acad Sci USA 90(17): 8033-7; Chen, C. M., D. M. Smith, et al. (1999). “Production and design of more effective avian replication-incompetent retroviral vectors.” Dev Biol 214(2): 370-84; Cosset et al (1991) “Improvements of Avian Leukosis Virus (ALV)-Based Retrovirus Vectors by Using Different cis-Acting Sequences from ALVs” J. of Virology 65(6): 3388-3394; Schaefer-Klein, J., I. Givol, et al. (1998). “The EV-O-derived cell line DF-1 supports the efficient replication of avian leukosis-sarcoma viruses and vectors.” Virology 248(2): 305-11; U.S. Pat. No. 6,096,534, issued Aug. 1, 2000; U.S. Pat. No. 5,672,485, issued Sep. 30, 1997; U.S. Pat. No. 5,985,642, issued Nov. 16, 1999; and U.S. Pat. No. 5,879,924, issued Mar. 9, 1999.

Any combination of features described herein is included within the scope of the present invention provided that the features included in any such combination are not mutually inconsistent. Such combinations will be apparent based on this specification and upon the knowledge of one of ordinary skill in the art.

### BRIEF DESCRIPTION OF THE FIGURES

FIG. 1 shows a map of pNLB-CMV-EPO (SEQ ID NO: 18) which contains the replication deficient pNLB vector coding sequence containing an expression cassette comprising a CMV promoter and an erythropoietin coding sequence (EPO 166 amino acids). In accordance with one aspect of the invention, the EPO coding sequence of pNLB-CMV-EPO can be substituted for a coding sequence desired for use in accordance with the invention. In addition, the CMV-EPO cassette of pNLB-CMV-EPO can be substituted for a promoter-coding sequence cassette desired for use in accordance with the invention.

FIG. 2 shows the helper pCMV-gagpol plasmid, the sequence of which is shown in SEQ ID NO: 17.

FIG. 3 shows the helper plasmid pVSV-G, the sequence of which is shown in SEQ ID NO: 16.

FIG. 4 shows schematic maps of vectors which can be useful in accordance with the invention. (A) The map of pNLB-CMV is shown. LTR, long terminal repeat; gag, partial coding sequence (CDS) of gag; SD, splice donor site; neo, neomycin or G418 resistance gene; SA, splice acceptor site; CMV, cytomegalovirus promoter; POI, CDS of protein of interest to be expressed; env, partial CDS of env; CTE, cytoplasmic transport element (Yang and Cullen (1999) RNA 5(12): 1645-55; and Paca, et al. (2000) J Virol 74(20): 9507-14). Bent arrows indicate potential transcription start sites. (B) Maps of the helper vectors used to package ALV-based vectors during transient transfections are shown. gag, complete CDS of the ALV gag protein; pol, complete CDS of the ALV pol protein (also shown in FIG. 2 and SEQ ID NO: 17); UTR, 3′ untranslated region of the bovine growth hormone gene; intron; intron of the rabbit β-globin gene; VSV G; G protein of the vesicular stomatitis virus (also shown in FIG. 3 and in SEQ ID NO 16); and alternative envelope protein encoding vector-envA, complete CDS of the ALV-A envelope protein which is well known in the art. (C) Map of pALV-SIN is shown. The restriction sites shown allowed cloning in of inserts except BpuAI which, when used, resulted in a low titer. Typically, the Nru-BamHI (BamHI site contained in the gag gene) fragment is removed and an expression cassette (coding sequence plus expression elements, e.g., promoter) is inserted at the site. It is expected that additional sequence can be removed between the gag BamHI site and the BpuA site without negatively effecting virus titer providing additional space to accommodate a larger cassette insert. A, ALV packaging signal; gag, partial coding sequence (CDS) of gag; env, partial CDS of env; SIN LTR, self-inactivating LTR. Maps are not drawn to scale. SEQ ID NO: 15 shows the sequence of pALV-SIN with a cassette sequence represented by a series of Ns. The sequence of pALV-SIN is shown in SEQ ID NO: 15. It is believed that in addition to the removal of the gag, pol and env genes, nucleotide sequence spanning from about 561 to 797 and/or 919 to 1130 of the vector as shown in SEQ ID NO: 15 can be removed to provide more room for insert DNA with having minimal or no negative effect on the vector as used in accordance with the present invention. (D) The structure of the ALV LTR is shown. The positions of the enhancer, CAAT box, TATA sequence and polyadenylation (pA) sequence are denoted by the solid horizontal line. The predicted transcription start site is indicated by the bent arrow. The U3, R and U5 regions of the intact ALV LTR as well as the sequence included in the SIN LTR of pALV-SIN are denoted by the boxes.

FIG. 5 shows a map of the expression vector pALV-SIN-4.2-Lys-IFNa-2B (SEQ ID NO: 19) which can be employed in accordance with the invention.

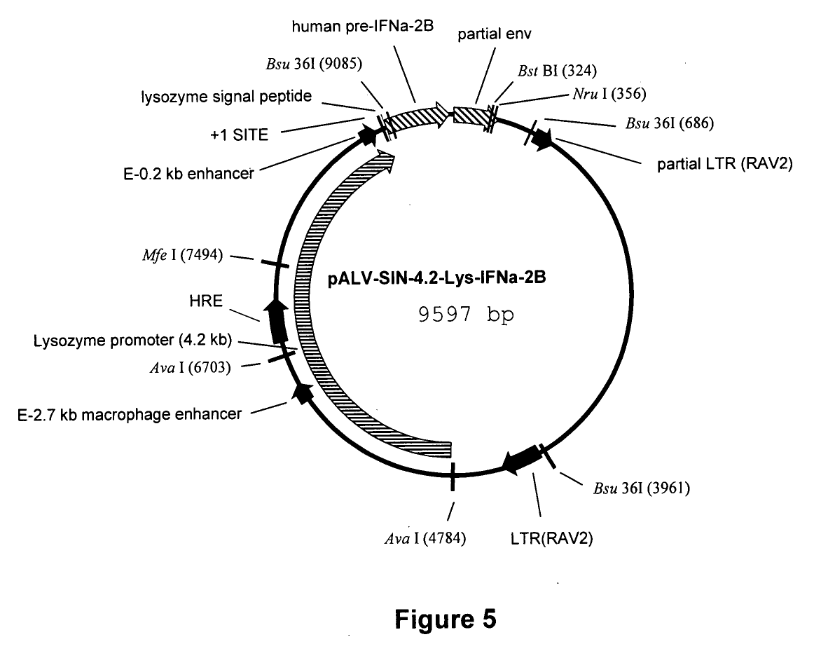

FIG. 6 shows a map of the expression vector pTombak (SEQ ID NO: 20) which contains a G-CSF coding sequence.

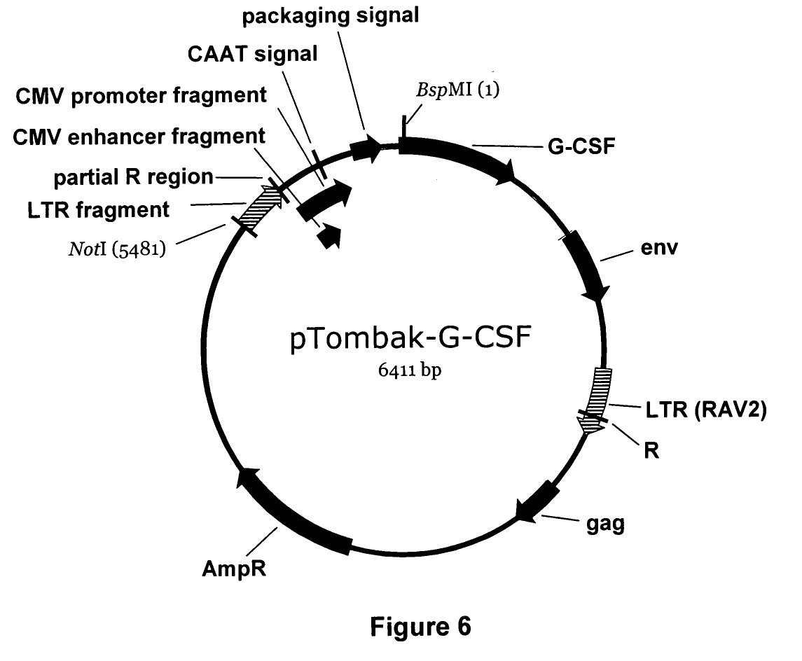

FIG. 7 shows the helper vector pCMV-gag-pol-SRD (SEQ ID NO: 21). SRD stands for Schmidt-Ruppin D stain of the Rous sarcoma virus. Bovie GH stands for bovine growth hormone.

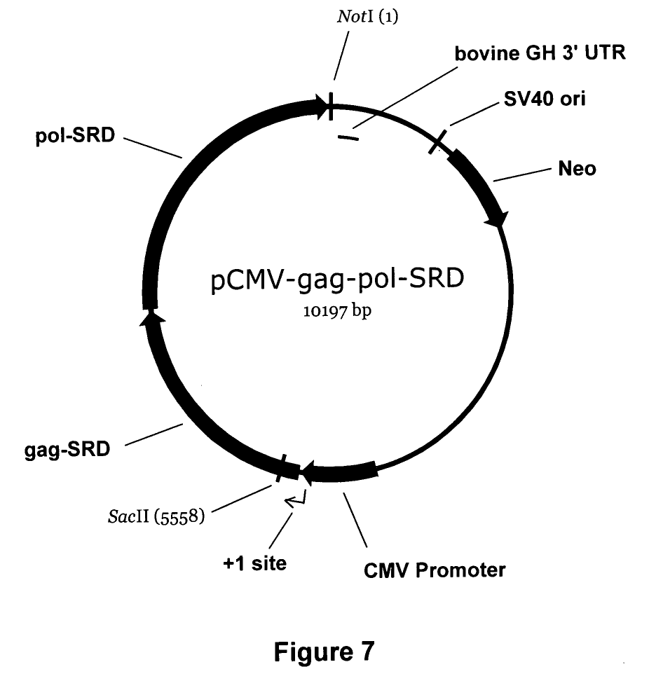

FIG. 8 shows a map of the helper vector pCMV-gag-pol-PRC (SEQ ID NO: 22). PRC stands for the Prague C strain of the Rous sarcoma virus.

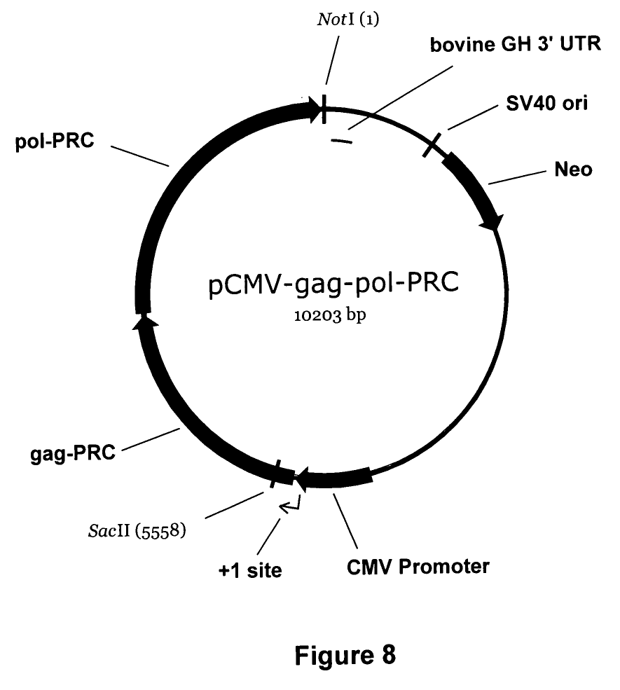

FIG. 9 shows a map of the helper vector pCMV-gag-SRD-pol-PRC (SEQ ID NO 23).

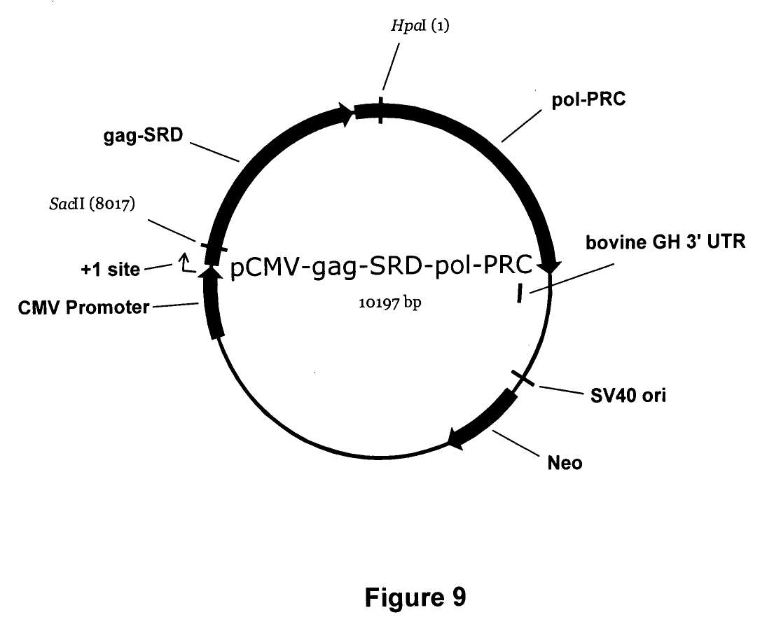

FIG. 10 shows a map of the helper vector p407-gag-SRD-pol-PRC (SEQ ID NO: 24). The CMV promoter fragment and LTR fragment form a fusion promoter.

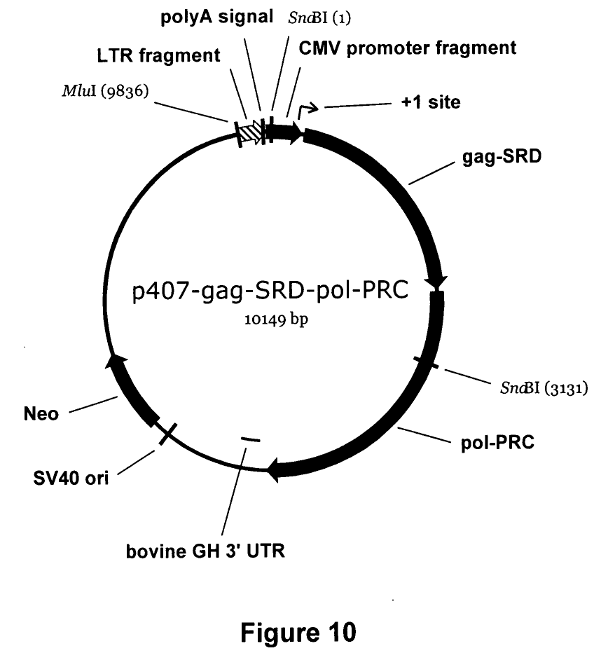

FIG. 11 shows a map of helper vector pLTRrev-CMV-gag-SRD-pol-PRC (SEQ ID NO: 25).

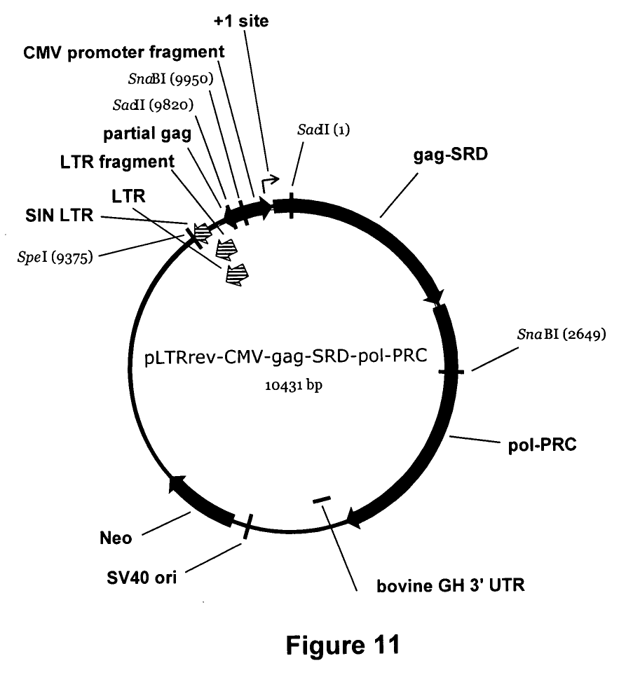

### DETAILED DESCRIPTION

Some of the definitions and abbreviations used herein include the following: aa, amino acid(s); bp, base pair(s); CDS, coding sequence cDNA, DNA complementary to an RNA; GalNac, N-acetylgalactosamine; Gal, galactose; GlcNac, IRES, internal ribosome entry site; N-acetylglucosamine nt, nucleotide(s); kb, 1000 base pairs; μg, microgram; ml, milliliter; ng, nanogram; nt, nucleotide.

Certain definitions are set forth herein to illustrate and define the meaning and scope of the various terms used to describe the invention herein.

The term “avian” as used herein refers to any species, subspecies or strain of organism of the taxonomic class ava, such as, but not limited to chicken, turkey, duck, goose, quail, pheasants, parrots, finches, hawks, crows and ratites including ostrich, emu and cassowary. The term includes the various known strains of Gallus gallus, or chickens, (for example, White Leghorn, Brown Leghorn, Barred-Rock, Sussex, New Hampshire, Rhode Island, Australorp, Minorca, Amrox, California Gray), as well as strains of turkeys, pheasants, quails, duck, ostriches and other poultry commonly bred in commercial quantities. It also includes an individual avian organism in all stages of development, including embryonic and fetal stages. The term “avian” also may denote “pertaining to a bird”, such as “an avian (bird) cell.”

The terms “avian retroviral vector” or “avian retrovirus” interchangeably refer to retro-transcribing viruses that primarily infect avians. Examples of avian retroviruses include, without limitation, Avian Leukemia/Leukosis Viruses (ALV), for example, and without limitation, RAV-0, RAV-1, RAV-2; Avian Sarcoma Viruses (ASV); Avian Sarcoma/Acute Leukemia Viruses (ASLV) including, without limitation, Rous Sarcoma Virus (RSV); Fujinami Sarcoma Viruses (FSV); Avian Myeloblastosis Viruses (AMV); Avian Erythroblastosis Viruses (AEV); Avian Myelocytomatosis Viruses (MCV), for example, and without limitation, MC29; Reticuloendotheliosis Viruses (REV), for example, and without limitation, Spleen Necrosis Virus (SNV).

The terms “Avian Leukemia Virus” or “Avian Leukosis Virus” or “ALV” interchangeably refer to a genus of alpha-retroviruses, which are members of Orthoretrovirinae, within the greater genus of Retroviridae, all of which are Retro-transcribing viruses. The complete genomic sequences of several ALV strains are known in the art and published, for example, as GenBank accession number EU070902, ALV strain PDRC-3249; EU070901, ALV strain PDRC-3246; EU070900, ALV strain PDRC-1039; NC_{—}001408; and AB303223, ALV strain TymS_{—}90.

A “nucleic acid or polynucleotide sequence or nucleotide sequence” includes, but is not limited to, mRNA, cDNA, genomic DNA, and synthetic DNA and RNA sequences, comprising the natural nucleoside bases adenine, guanine, cytosine, thymidine, and uracil. The term also encompasses sequences having one or more modified bases such as, without limitation, pseudo uridine, 2-amino purine, doeoxy uridine and deoxyinosine.

“Therapeutic proteins” or “pharmaceutical proteins” include an amino acid sequence which in whole or in part makes up a drug.

“Transgene” is a DNA sequence inserted into a genome, i.e., an exogenous DNA sequence. A transgene may refer to the entire sequence that is inserted, for example, the inserted retrovirus plus any sequences carried by the retrovirus.

“Transgene” may also refer to the sequence of interest carried by the retrovirus, for example, a coding sequence and promoter or, for example, the nucleotide sequence between the LTRs of the inserted retrovirus.

The phrases “based on” or “based upon” as in a retroviral vector being based on a particular retrovirus or based on a nucleotide sequence of a particular retrovirus mean that the genome of the retroviral vector contains at least a substantial portion of or shares substantial sequence identity with the nucleotide sequence of the genome of the particular reference retrovirus. The substantial portion can include coding and/or non-coding nucleic acid sequences. In some embodiments, the retroviral vector being based on a particular retrovirus contains at least 60%, e.g., at least about 70%, 75%, 80%, 85%, 90%, 95% or all of the retrovirus genome, as will be apparent from the context in the specification and the knowledge of one skilled in the art. In one embodiment, retroviral vectors of the invention are based on an avian retrovirus, e.g., ALV, and contain a modified retrovirus genome that does not encode one or more viral polypeptides e.g., gag, pol, and/or env, and has transcriptionally inactive 3′LTR and 5′LTR. In some embodiments, the retroviral vector being based on a particular retrovirus contains at least 90%, 91%, 92%, 93%, 94%, 95%, 96%, 97%, 98% or 99% sequence identity with a reference retrovirus, e.g., an ALV retrovirus of SEQ ID NO: 15 or having GenBank Accession No. EU070902, EU070901, EU070900, NC_{—}001408 or AB303223. Examples of retroviral vectors that are based on a retrovirus include the NL retroviral vectors (e.g., NLB) which are based on the ALV retrovirus as disclosed in Cosset et al, Journal of Virology (1991) (65):3388-3394. NL vectors including NLB, NLD and NLA are contemplated for use in methods of the present invention.

A “coding sequence” or “open reading frame” refers to a nucleotide sequence which can be transcribed and translated (in the case of DNA) or translated (in the case of mRNA) into a polypeptide in vitro or in vivo when placed under the control of appropriate regulatory sequences. The boundaries of the coding sequence are determined by a translation start codon at the 5′ (amino) terminus and a translation stop codon at the 3′ (carboxy) terminus. A transcription termination sequence will usually be located 3′ to the coding sequence. A coding sequence may be flanked on the 5′ and/or 3′ ends by untranslated regions.

Nucleic acid “controlling sequences” or “regulatory sequences” refer to promoter sequences, translational start and stop codons, ribosome binding sites, polyadenylation signals, transcription termination sequences, upstream regulatory domains, enhancers, and the like, as necessary and sufficient for the transcription and translation of a given coding sequence in a defined host cell. Examples of control sequences suitable for eukaryotic cells are promoters, polyadenylation signals, and enhancers. All of these control sequences need not be present in a recombinant vector so long as those necessary and sufficient for the transcription and translation of the desired sequence are present.

The term “cytokine” as used herein refers to any secreted amino acid sequence that affects the functions of cells and is a molecule that modulates interactions between cells in the immune, inflammatory or hematopoietic responses. A cytokine includes, but is not limited to, monokines and lymphokines regardless of which cells produce them. For instance, a monokine is generally referred to as being produced and secreted by a mononuclear cell, such as a macrophage and/or monocyte. Many other cells however also produce monokines, such as natural killer cells, fibroblasts, basophils, neutrophils, endothelial cells, brain astrocytes, bone marrow stromal cells, epideral keratinocytes and B-lymphocytes. Lymphokines are generally referred to as being produced by lymphocyte cells. Examples of cytokines include, but are not limited to, interferon, erythropoietin, G-CSF, Interleukin-1 (IL-1), Interleukin-6 (IL-6), Interleukin-8 (IL-8), Tumor Necrosis Factor-alpha (TNF-alpha) and Tumor Necrosis Factor beta (TNF-beta).

“Operably or operatively linked” refers to the configuration of the coding and control sequences so as to perform the desired function. Thus, control sequences operably linked to a coding sequence are capable of effecting the expression (i.e., transcription and/or translation) of the coding sequence. In one embodiment, a coding sequence is operably linked to or under the control of transcriptional regulatory regions in a cell when DNA polymerase will bind the promoter sequence and transcribe the coding sequence into mRNA that can be translated into the encoded protein. The control sequences need not be contiguous with the coding sequence, so long as they function to direct the expression thereof. Thus, for example, intervening untranslated yet transcribed sequences can be present between a regulatory sequence and the coding sequence and the regulatory sequence can still be considered “operably linked” to the coding sequence.

The term “isolated nucleic acid” as used herein covers, for example, (a) a DNA which has the sequence of part of a naturally occurring genomic molecule but is not flanked by at least one of the sequences that flank that part of the molecule in the genome of the species in which it naturally occurs; (b) a nucleic acid which has been incorporated into a vector or into the genomic DNA of a prokaryote or eukaryote in a manner such that the resulting vector or genomic DNA is not identical to naturally occurring DNA from which the nucleic acid was obtained; (c) a separate molecule such as a cDNA, a genomic fragment, a fragment produced by polymerase chain reaction (PCR), ligase chain reaction (LCR) or chemical synthesis, or a restriction fragment; (d) a recombinant nucleotide sequence that is part of a hybrid gene, i.e., a gene encoding a fusion protein, and (e) a recombinant nucleotide sequence that is part of a hybrid sequence that is not naturally occurring. Isolated nucleic acid molecules of the present invention can include, for example, natural allelic variants as well as nucleic acid molecules modified by nucleotide deletions, insertions, inversions, or substitutions.

The term “vector” and “nucleic acid vector” as used herein refers to a natural or synthetic single or double stranded plasmid or viral nucleic acid molecule that can be transfected or transformed into cells and replicate independently of, or within, the host cell genome. A circular double stranded vector can be linearized by treatment with an appropriate restriction enzyme based on the nucleotide sequence of the vector. A nucleic acid can be inserted into a vector by cutting the vector with restriction enzymes and ligating the desired pieces together, as is understood in the art.

The term “oviduct specific promoter” as used herein refers to promoters and promoter components which are functional, e.g., provide for transcription of a coding sequence, to a large extent, for example, primarily (i.e., more than 50% of the transcription product produced in the animal by a particular promoter type being produced in oviduct cells) or exclusively in oviduct cells of a bird. Examples of oviduct specific promoters include, ovalbumin promoter, ovomucoid promoter, ovoinhibitor promoter, lysozyme promoter and ovotransferrin promoter and functional portions of these promoters, e.g., promoter components.

The terms “percent sequence identity”, “percent identity”, “% identity”, “percent sequence homology”, “percent homology”, “% homology” and “percent sequence similarity” each refer to the degree of sequence matching between two nucleic acid sequences or two amino acid sequences. Such sequence matching can be determined using the algorithm of Karlin & Attschul (1990) Proc. Natl. Acad. Sci. 87: 2264-2268, modified as in Karlin & Attschul (1993) Proc. Natl. Acad. Sci. 90: 5873-5877. Such an algorithm is incorporated into the NBLAST and XBLAST programs of Attschul et al. (1990) T. Mol. Biol. Q15: 403-410. BLAST nucleotide searches are performed with the NBLAST program, score=100, wordlength=12, to obtain nucleotide sequences homologous to a nucleic acid molecule of the invention. BLAST protein searches are performed with the XBLAST program, score=50, wordlength=3, to obtain amino acid sequences homologous to a reference amino acid sequence. To obtain gapped alignments for comparison purposes, Gapped BLAST is utilized as described in Attschul et al. (1997) Nucl. Acids Res. 25: 3389-3402. When utilizing BLAST and Gapped BLAST programs, the default parameters of the respective programs (e.g. XBLAST and NBLAST) are used. Other algorithms, programs and default settings may also be suitable such as, but not only, the GCG-Sequence Analysis Package of the U.K. Human Genome Mapping Project Resource Centre that includes programs for nucleotide or amino acid sequence comparisons.

As used herein, the terms “exogenous”, “heterologous” and “foreign” with reference to nucleic acids, such as DNA and RNA, are used interchangeably and refer to nucleic acid that does not occur naturally as part of a chromosome, a genome or cell in which it is present or which is found in a location(s) and/or in amounts that differ from the location(s) and/or amounts in which it occurs in nature. It can be nucleic acid that is not endogenous to the genome, chromosome or cell and has been exogenously introduced into the genome, chromosome or cell. Examples of heterologous DNA include, but are not limited to, DNA that encodes a gene product or gene product(s) of interest, for example, for production of an encoded protein. Examples of heterologous DNA include, but are not limited to, DNA that encodes traceable marker proteins, DNA that encodes therapeutic proteins. The terms “heterologous” and “exogenous” can refer to a biomolecule such as a nucleic acid or a protein which is not normally found in a certain cell, tissue or substance produced by an organism or is not normally found in a certain cell, tissue or substance produced by an organism in an amount or location the same as that found to occur naturally. For example, a protein that is heterologous or exogenous to an egg is a protein that is not normally found in the egg.

The expression products described herein may consist of proteinaceous material having a defined chemical structure. However, the precise structure depends on a number of factors, particularly chemical modifications common to proteins. For example, since all proteins contain ionizable amino and carboxyl groups, the protein may be obtained in acidic or basic salt form, or in neutral form. The primary amino acid sequence may be derivatized using sugar molecules (glycosylation) or by other chemical derivatizations involving covalent or ionic attachment with, for example, lipids, phosphate, acetyl groups and the like, often occurring through association with saccharides. These modifications may occur in vitro, or in vivo, the latter being performed by a host cell through posttranslational processing systems. Such modifications may increase or decrease the biological activity of the molecule, and such chemically modified molecules are also intended to come within the scope of the invention.

A “retroviral vector” is a retrovirus or a modified retrovirus or virus that can be used to shuttle nucleotide sequences into a cell. The term virus, viral vector, retrovirus, retroviral vector, particle and retroviral particle may be used interchangeably throughout the specification. Typically, retroviral vectors employed in the invention are replication deficient.

A “promoter” is a site on the DNA to which RNA polymerase binds to initiate transcription of a gene. In some embodiments the promoter will be modified by the addition or deletion of sequences, or replaced with alternative sequences, including natural and synthetic sequences as well as sequences which may be a combination of synthetic and natural sequences. Many eukaryotic promoters contain two types of recognition sequences: the TATA box and the upstream promoter elements. The former, located upstream of the transcription initiation site, is involved in directing RNA polymerase to initiate transcription at the correct site, while the latter appears to determine the rate of transcription and is typically upstream of the TATA box. Enhancer elements can also stimulate transcription from linked promoters, but many function exclusively in a particular cell type. Many enhancer/promoter elements derived from viruses, e.g., the SV40 promoter, the cytomegalovirus (CMV) promoter, the rous-sarcoma virus (RSV) promoter, and the murine leukemia virus (MLV) promoter are all active in a wide array of cell types, and are termed “constitutive” or “ubiquitous”. An example of a non-constitutive promoter is the mouse mammary tumor virus (MMTV) promoter. The nucleic acid sequence inserted in the cloning site may have any open reading frame encoding a polypeptide of interest, with the proviso that where the coding sequence encodes a polypeptide of interest, it should lack cryptic sites which can block production of appropriate mRNA molecules and/or produce aberrantly spliced or abnormal mRNA molecules.

Examples of promoters for expression of therapeutic polypeptides in the present retrovirus-based vectors include constitutive, inducible and tissue specific. Constitutive promoters can include but are not limited to CMV, SV40, RSV and MLV promoters.

Inducible promoters can include but are not limited to metalloproteinase promoters (e.g., MMP-1 and MMP-9 promoters), radiation sensitive promoters (e.g., the early growth response-1 gene CArG elements and tissue plasminogen activator (tPA) promoter), glucose regulated promoters (e.g., the GRP78/BiP promoter), hypoxia regulated promoters (e.g., the hypoxia enhancer or hypoxia response element (HRE) sequence) and bacterial regulatory sequences (e.g., the tetracycline repressor and the T7 promoter).

Tissue specific promoters, which can be constitutive promoters (e.g., oviduct specific promoters), include prostate cell specific promoters (e.g., prostate specific antigen (PSA) promoter), liver cell/hepatocyte specific promoters (e.g., the alpha-fetoprotein promoter and the albumin enhancer element), endothelial cell specific promoters (e.g., the von Willebrand factor (vWf) promoter and the tie-2/tek promoter), breast cancer specific promoters (e.g., the DF3 (MUC1) promoter and the HER-2/neu promoter), melanocyte specific promoters (include the tyrosinase gene 5′ region), glioma cell specific (e.g., the myelin basic promoter) and pancreatic cancer specific promoters (e.g., the human carcinoembryonic antigen (CEA) promoter). Nucleotide sequences that target expression to specific cell states can also be used, for example the four repeats of the Myc-Max response element can target expression to Myc overexpressing cancer cells. Many promoters are well known in the art and are contemplated by the methods of the present invention. Any promoter including but not limited to those listed above can be incorporated into the primary nucleic acid cassette operably linked with the gene expressing the desired protein.

“Magnum” is that part of the oviduct between the infundibulum and the isthmus containing tubular gland cells that synthesize and secrete the egg white proteins of the egg.

A “marker gene” is a gene which encodes a protein which can allow for identification of transfected cells. Suitable marker sequences include, but are not limited to green, yellow, and blue fluorescent protein genes (GFP, YFP, and BFP, respectively). Other suitable markers include thymidine kinase (tk), dihydrofolate reductase (DHFR), and aminoglycoside phosphotransferase (APH) genes. The latter imparts resistance to the aminoglycoside antibiotics, such as kanamycin, neomycin, and geneticin. These, and other marker genes such as those encoding chloramphenicol acetyltransferase (CAT), β-lactamase, β-galactosidase (β-gal), may be incorporated into the primary nucleic acid cassette along with the gene expressing the desired protein, or the selection markers may be contained in separate vectors and cotransfected.

The term “monogenic disease” refers to an inherited disease or condition that results from inactivation or malfunctioning of a single gene (e.g., due to mutation or deletion) occurring in cells, e.g., all cells, in an individual.

The term “optimized” is used in the context of “optimized coding sequence”, wherein the most frequently used codons for each particular amino acid found in a protein, for example, in an egg white protein such as ovalbumin, lysozyme, ovomucoid, and ovotransferrin are used in the design of optimized polynucleotide sequence, encoding exogenous protein, that can be inserted into retroviral vectors or particles produced according to the present invention. More specifically, the optimized DNA sequence is based on the hen oviduct optimized codon usage and may be produced using the BACKTRANSLATE program of the Wisconsin Package, Version 9.1 (Genetics Computer Group Inc., Madison, Wis.) with a codon usage table compiled from the chicken (Gallus gallus) ovalbumin, lysozyme, ovomucoid, and ovotransferrin proteins. For example, the percent usage for the four codons of the amino acid alanine in the four egg white proteins is 34% for GCU, 31% for GCC, 26% for GCA, and 8% for GCG.

The term “plasmid” as used herein typically refers to a vector that cannot reproduce in a eukaryotic cell and typically does not integrate into the genome of a eukaryotic cell. Plasmids are useful in producing transient transfection.

A “reporter gene” is a marker gene that “reports” its activity in a cell by the presence of the protein that it encodes.

A “replication deficient” virus or viral vector is a virus or viral vector that is missing an element from its genome that is required for replication.

A “retroviral particle”, “transducing particle”, and “transduction particle” refer to a replication-defective or replication-competent virus or retrovirus capable of transducing non-viral DNA or RNA into a cell.

A “replication-deficient and self-inactivating ALV vector” typically has two or more of the gag, pol and env genes deleted from its genome and is a self inactivating vector. In a particularly useful embodiment, a replication-deficient and self-inactivating ALV vector has each of the gag, pol and env gene delete from its genome and is a self inactivating vector.

A “SIN vector” is a self-inactivating vector. In particular, a SIN vector is a retroviral vector having an altered genome such that upon integration into genomic DNA of the target cell (e.g., avian embryo cells) the 5′ LTR of the integrated retroviral vector will not function as a promoter. For example, a portion or all of the nucleotide sequence of the retroviral vector that results in the U3 region of the 5′ LTR of the retroviral vector once integrated may be deleted or altered in order to reduce or eliminate promoter activity of the 5′ LTR. In certain examples, deletion of the CAAT box and/or the TAATA box from U3 of the 5′ LTR can result in a SIN vector, as is understood in the art.

A “therapeutic protein” or “pharmaceutical protein” is a substance that, in whole or in part, makes up a drug. In particular, “therapeutic proteins” and “pharmaceutical proteins” include an amino acid sequence which in whole or in part makes up a drug.

The term “therapeutically effective amount” refers to an amount of viral vector which provides either subjective relief of a symptom(s) or an objectively identifiable improvement, e.g., inhibition of tumor-cell growth, as noted by a clinician or other qualified observer. The dosing range varies with the viral vector used, the route of administration and the potency of the particular viral vector. A therapeutically effective amount is a sufficient dose to provide an efficacious effect while minimizing or avoiding undesirable side effects.

The terms “transformation”, “transduction” and “transfection” all denote the introduction of a polynucleotide into a cell.

As used herein, a “transgenic animal” is any non-human animal, such as an avian species, including the chicken, in which one or more of the cells of the animal contain heterologous nucleic acid introduced by way of human intervention, such as by transgenic techniques known in the art (see, for example, US patent publication No. 2007/0243165, published Oct. 18, 2007, the disclosure of which is incorporated in its entirety herein by reference) including those disclosed herein. The nucleic acid is introduced into an animal, directly or indirectly by introduction into a cell (e.g., egg or embryo cell) by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animal, the transgene can cause cells to express a recombinant form of the target protein or polypeptide. The terms “chimeric animal” or “mosaic animal” are used herein to refer to animals in which a transgene is found, or in which the recombinant nucleotide sequence is expressed, in some but not all cells of the animal. A germ-line chimeric animal contains a transgene in its germ cells and can give rise to an offspring transgenic animal in which most or all cells of the offspring will contain the transgene.

As used herein, the term “transgene” means a nucleic acid sequence (encoding, for example, a human protein) that is partly or entirely heterologous, i.e., foreign, to the animal or cell into which it is introduced, or, is partly or entirely homologous to an endogenous gene of the transgenic animal or cell into which it is introduced, but which is designed to be inserted, or is inserted, into the animal or cell genome in such a way as to alter the genome of the organism into which it is inserted (e.g., it is inserted at a location which differs from that of the natural gene or its insertion results in a knockout).

“Transduce refers to a viral vector of the invention integrating into a cell's genome. “Transfect” refers to introduction of DNA sequence into a cell.

“Vector” means a polynucleotide comprised of single strand, double strand, circular, or supercoiled DNA or RNA. A typical vector may include the following elements operatively linked at appropriate distances for allowing functional gene expression: replication origin, promoter, enhancer, 5′ mRNA leader sequence, ribosomal binding site, nucleic acid cassette, termination and polyadenylation sites, and selectable marker sequences. One or more of these elements may be omitted in specific applications. The nucleic acid cassette can include one or more restriction sites for insertion of the nucleic acid sequence to be expressed. In a functional vector the nucleic acid cassette contains the nucleic acid sequence to be expressed including translation initiation and termination sites. An intron optionally may be included in the construct, for example, 5′ to the coding sequence. A vector is constructed so that the particular coding sequence is located in the vector with the appropriate regulatory sequences, the positioning and orientation of the coding sequence with respect to the control sequences being such that the coding sequence is transcribed under the “control” of the controlling or regulatory sequences. Modification of the sequences encoding the particular protein of interest may be desirable to achieve this end. For example, in some cases it may be necessary to modify the sequence so that it may be attached to the control sequences with the appropriate orientation; or to maintain the reading frame. The control sequences and other regulatory sequences may be ligated to the coding sequence prior to insertion into a vector. Alternatively, the coding sequence can be cloned directly into an expression vector which already contains the control sequences and an appropriate restriction site which is in reading frame with and under regulatory control of the control sequences.

In one aspect, the invention is directed to producing viral particles capable of introducing nucleotide sequences into cells, for example, avian cells, including embryonic cells. For example, replication deficient retroviral vectors can be produced in accordance with the invention.

The invention contemplates the application of any useful cell to be employed in accordance with the present invention, such as avian cells. In one particularly useful embodiment, the cells used herein are immortal; that is, the cells are capable of continuous growth in culture.

Fibroblast cells (i.e., fibroblast cell lines) have shown to be particularly useful as disclosed herein, though the invention is not limited thereto. For example, the invention contemplates the use of human fibroblast cells, rabbit fibroblast cells, bovine fibroblast cells, reptile fibroblast cells, fibroblast cells from fishes or other useful fibroblast cells. In one particularly useful aspect of the invention, avian fibroblast cells are employed. The invention is not limited to the use of any particular avian fibroblast cells; however, examples of avians from which fibroblast cells may be derived for use in accordance with the invention include, without limitation, turkeys, ducks, geese, quail, pheasants, parrots, finches, hawks, crows and ratites including ostrich, emu and cassowary. One particularly useful type of avian fibroblast cell for use as disclosed herein is the chicken fibroblast cell. Fibroblast cells of any variety of chicken (i.e., Gallus gallus), such as, but not limited to, White Leghorn, Brown Leghorn, Barred-Rock, Sussex, New Hampshire, Rhode Island, Australorp, Minorca, Amrox and California Gray can be used.

Fibroblast cells typically are cells present in or cells that give rise to connective tissue. In one aspect, fibroblast cells are cells that give rise to collagen. Fibroblast cells may be defined as cells that secrete an extracellular matrix rich in collagen. Fibroblast cells may be derived from a variety of sources. For example, the invention contemplates fibroblast cells obtained from tissue such as muscle tissue and from organs such as the liver, skin and lungs. In one embodiment, the invention contemplates the use of embryo fibroblast cells such as chicken embryo fibroblast cells, for example, immortal chicken embryo fibroblast cell lines. A particularly useful fibroblast cell line (DF-1) is disclosed in U.S. Pat. No. 5,672,485, issued Sep. 30, 1997, the disclosure of which is incorporated in its entirety herein by reference.

The invention contemplates the introduction of certain nucleotide sequences into cells; i.e., nucleotide sequences encoding replication deficient retroviruses and nucleotide sequences that encode products required for replication of the replication deficient retrovirus, for example, two or more of gag, pol and env proteins. The products required are typically biomolecules that are necessary for replication or propagation of the retrovirus. For example, and without limitation, proteins required for replication or propagation of the retrovirus can be one or more of: viral polymerase; one or more proteins contained in the viral envelope; one or more proteins contained in the capsid.

The nucleotide sequences introduced into the cells may be in any useful form. For example, the nucleotide sequences may be DNA or RNA. The nucleotide sequences introduced into the cells may be in linear form or circular form. In one embodiment, the nucleotide sequences are contained in a circular vector.

Any useful avian retroviral vector may be employed in the present invention. In one embodiment, avian retroviral vectors of the invention are not designed to integrate into the genome of cells used for there production and are also designed not to replicate inside of the cell. Many commercially available vectors such as plasmids or phagemids are available that can be used in accordance with the invention, such as pBluescript®, pBR322, pUC19, pDRIVE and others.

In one embodiment, the nucleotide sequences are transiently introduced into the cell by any useful method. For example, the nucleotide sequences may be introduced into the cells using, for example, electroporation, calcium phosphate precipitation, microinjection, sonication, microparticle bombardment as well as using drendrimers, PEI, polylysine and polyamine and other techniques, each as is understood by a practitioner of skill in the art. One particularly useful method of introducing the nucleotide sequences into the cells is by transfection, for example, lipofection. Methods of transfecting cells by lipofection are well known in the art. Examples of lipofection reagents that can be used in accordance with the invention include, without limitation, DMRIE C, FuGENE and Lipofectamine™.

By the methods of the present invention, transgenes contained in viral particles produced in accordance with the present invention, can be introduced into avian embryonic blastodermal cells, to produce a transgenic chicken, transgenic turkey, transgenic quail and other avian species, that carries the transgene in the genetic material of its germ-line tissue. The blastodermal cells may be stage I to XII cells, or the equivalent thereof, and are typically near stage X (e.g., stage VII to stage XII). Retroviral particles produced as disclosed herein are also contemplated for use in transducing primordial germ cells from later stage embryos, including embryos from stage 13 to stage 30. Typically, though not exclusively, the blastodermal cells are present inside of a hard shell egg. The cells useful for producing transgenic avians include cells termed embryonic blastodermal (EB) cells, embryonic germ (EG) cells, embryonic stem (ES) cells & primordial germ cells (PGCs). It is contemplated that the embryonic blastodermal cells may be isolated freshly, maintained in culture, or, in a particularly useful embodiment, reside in situ within an embryo.

Examples of viral particles which can be produced in accordance with the invention include replication deficient viral particles that contain a coding sequence for a useful protein which is linked to a promoter that provides for expression of the useful protein in a host cell, for example, a cell of a transgenic animal. For example, the useful protein can be a human protein or other useful protein such as those disclosed herein. In one embodiment, the viral particles may be used to produce exogenous proteins in specific tissues of an avian, for example, in the oviduct tissue of an avian. In a particularly useful embodiment, the viral particles are used in methods to produce avians that lay eggs which contain exogenous protein.

In one embodiment of the invention, an avian retroviral vector such as an ALV based vector such as NLB is cotransfected into a fibroblast cell line (e.g., a chicken fibroblast cell line) such as DF-1 cells (e.g., via lipofection) along with a pCMV-gag-pol-SRD expression vector and a third vector which expresses an envelope protein, for example, an envelope protein of the vesicular stomatitis virus (VSV-G) or of ALV (envA). After 48 hours, the media is harvested and contains high titer ALV based retroviral particles. The virus particles can be concentrated by centrifugation to achieve even higher titers. In certain embodiments, the cells are treated with sodium butyrate which provides for a further increase in viral titer.

In one particular embodiment of the invention, an avian retroviral vector such as an ALV based vector such as NLB is cotransfected into a fibroblast cell line (e.g., a chicken fibroblast cell line) such as DF-1 cells (e.g., via lipofection) along with a rous sarcoma virus (RSV) gag-pol expression vector and a third vector which expresses an envelope protein, for example, an envelope protein of the vesicular stomatitis virus (VSV-G) or of ALV (envA). After 48 hours, the media is harvested and contains high titer ALV based retroviral particles. The virus particles can be concentrated by centrifugation to achieve even higher titers. In certain embodiments, the cells are treated with sodium butyrate which provides for a further increase in viral titer.

In one embodiment, in the genome of the viral particles produced as disclosed herein, the exogenous protein coding sequence and the promoter are both positioned between 5′ LTR and the 3′ LTR. The vector may include a marker nucleotide sequence, wherein the marker nucleotide sequence is operably linked to a promoter.

In one embodiment, the viral vectors produced in accordance with the invention include a signal peptide coding sequence which is operably linked to the exogenous protein coding sequence, so that upon translation in a cell, the signal peptide will direct secretion of the exogenous protein expressed by the vector into the egg white and the exogenous protein will be packaged into a hard shell egg.

In certain embodiments, introduction of a vector of the present invention into the embryonic blastodermal cells is performed with embryonic blastodermal cells that are either freshly isolated or in culture. The transgenic cells are then typically injected into the subgerminal cavity beneath a recipient blastoderm in an egg. In some cases, however, the vector is delivered directly into the subgerminal cavity of a blastodermal embryo in situ.

In one embodiment of the invention, viral particles used for transfecting blastodermal cells and generating stable integration in the avian genome contain a coding sequence and a promoter in operational and positional relationship to express the coding sequence in the tubular gland cell of the magnum of the avian oviduct, wherein the coding sequence codes for an exogenous protein which is deposited in the egg white of a hard shell egg. The promoter may be a portion of a promoter that is particularly active (i.e., highly expressed) in tubular gland cells such as the ovalbumin promoter, ovomucoid promoter or lysozyme promoter. The invention contemplates truncating such promoters and/or condensing the critical regulatory elements of the promoters so that it retains sequences required for expression in the tubular gland cells of the magnum of the oviduct, while being small enough that it can be readily incorporated into genome of the viral particles. The invention also contemplates the use of a fusion promoter. In another particularly useful embodiment, the promoter is a constitutive promoter, for example, and without limitation, a cytomegalovirus (CMV) promoter, a rous-sarcoma virus (RSV) promoter, a murine leukemia virus (MLV) promoter or a beta-actin promoter, a murine leukemia virus (MLV) promoter, a LTR promoter.

If desired, transducing particles (i.e., transduction particles) produced in accordance with the invention can be titered by any useful method as is understood by a practitioner of skill in the art. For example, if the viral genome contains a marker such as a neomycin resistance gene, the particles can be titered by transduction of cells and serial dilution followed by plating and counting of colonies. In one embodiment, the titer is determined by hybridization to the vial genome (e.g., quantitative densitometry of a probed blot of the viral nucleic acid (RNA or DNA) as is understood by practitioners of skill in the art). Immunofluorescence or ELISA analysis to quantitate viral coat protein and quantitative PCR of the viral genome, for example, quantitative PCR of the reverse transcription product from the viral genome can also be used. In one embodiment, titer is not determined before use of the viral particles.

In one embodiment, viral particles of the invention are introduced into avian blastodermal cells by egg windowing methods, for example, in accordance with the Speksnijder procedure (U.S. Pat. No. 5,897,998). That is, the viral particles are introduced into the blastodermal cells in situ, for example, by introduction into the subgerminal cavity of the embryo. After introduction (e.g., injection), the eggs hatch after about 21 days. Typically, male birds are selected for breeding. In order to screen for G0 roosters which contain the transgene (e.g., introduced nucleotide sequence) in their sperm, DNA is extracted from rooster sperm samples. The G0 roosters with the highest levels of the transgene in their sperm samples can be bred to nontransgenic hens by artificial insemination. Blood DNA samples are screened for the presence of the transgene and in the case of avians produced for exogenous protein production, the blood may be assayed (e.g., ELISA) for the exogenous protein. If presence of the exogenous protein is confirmed, the sperm of the G1 transgenic roosters can be used for artificial insemination of nontransgenic hens. A certain percent of the G2 offspring will contain the transgene (e.g., about 50%).

Transgenic avians produced from the blastodermal cells are known as founders. Some founders will carry the transgene in the tubular gland cells in the magnum of their oviducts. These avians can express the exogenous protein encoded by the transgene in their oviducts. The exogenous protein may also be present in other tissues (e.g., blood) in addition to the oviduct. If the exogenous protein contains the appropriate signal sequence(s), it may be secreted into the lumen of the oviduct and into the egg white of the egg. Some founders are germ-line founders. A germ-line founder is a founder that carries the transgene in genetic material of its germ-line tissue, and may or may not carry the transgene in tubular gland cells which express the exogenous protein. Therefore, in accordance with the invention, the transgenic avian may have tubular gland cells expressing the exogenous protein. Regardless if the founder contains the genetic material in its tubular gland cells, if the founder is a germ-line founder some of its offspring will be completely transgenic (i.e., not chimeric) and will have tubular gland cells that express the exogenous protein. In certain embodiments, the offspring can express a phenotype determined by expression of the exogenous gene in only specific tissue(s) of the avian, for example, by use of a tissue specific promoter.

In one specific example, for the production of transgenic chickens as disclosed herein, a CMV promoter was linked to the coding sequence of erythropoietin (165 amino acid form; see, for example, Pharmacotherapy (1990) Supplement to vol 10, No. 2, p 3S to 8S, the disclosure of which is incorporated in its entirety herein by reference) to form a cassette which was inserted into an ALV vector. The retroviral vector was produced transiently and concentrated to approximately 1×10⁷ particles/ml. 3 to 7 ul of concentrated virus was injected in the subgerminal cavity of windowed Charles River SPF line 21 unincubated eggs. Chicks were hatched and raised to sexual maturity. Males were screened for the presence of the transgene in their sperm DNA by quantitative PCR for the gene of interest, in this case EPO.

In one embodiment, the retroviral particles produced as disclosed herein are used to produce transgenic avians used to express, in large yields and at low cost, a wide range of desired proteins including those used as human and animal pharmaceuticals, diagnostics, and livestock feed additives. For example, the invention includes transgenic avians that produce such proteins and eggs laid by the transgenic avians which contain the protein, for example, in the egg white. The present invention is contemplated for use in the production of any desired protein including pharmaceutical proteins with the requisite that the coding sequence of the protein can be introduced into an oviduct cell in accordance with the present invention. In one particularly useful embodiment, the proteins produced as disclosed herein are human proteins, i.e., proteins produced by humans.

The invention, therefore, includes methods for producing multimeric proteins including immunoglobulins, such as antibodies, and antigen binding fragments thereof. Thus, in one embodiment of the present invention, the multimeric protein is an immunoglobulin, wherein the first and second heterologous polypeptides are immunoglobulin heavy and light chains respectively

In certain embodiments, an immunoglobulin polypeptide encoded by the transcriptional unit of at least one expression vector may be an immunoglobulin heavy chain polypeptide comprising a variable region or a variant thereof, and may further comprise a D region, a J region, a C region, or a combination thereof. An immunoglobulin polypeptide produced as disclosed herein may also be an immunoglobulin light chain polypeptide comprising a variable region or a variant thereof, and may further comprise a J region and a C region. The present invention also contemplates multiple immunoglobulin regions that are derived from the same animal species, or a mixture of species including, but not only, human, mouse, rat, rabbit and chicken. In certain embodiments, the antibodies are human or humanized.

In other embodiments, the immunoglobulin polypeptide produced as disclosed herein comprises an immunoglobulin heavy chain variable region, an immunoglobulin light chain variable region, and a linker peptide thereby forming a single-chain antibody capable of selectively binding an antigen.

Examples of therapeutic antibodies that may be produced in methods of the invention include but are not limited to HERCEPTIN™ (Trastuzumab) (Genentech, CA) which is a humanized anti-HER2 monoclonal antibody for the treatment of patients with metastatic breast cancer; REOPRO™ (abciximab) (Centocor) which is an anti-glycoprotein IIb/IIIa receptor on the platelets for the prevention of clot formation; ZENAPAX™ (daclizumab) (Roche Pharmaceuticals, Switzerland) which is an immunosuppressive, humanized anti-CD25 monoclonal antibody for the prevention of acute renal allograft rejection; PANOREX™ which is a murine anti-17-IA cell surface antigen IgG2a antibody (Glaxo Wellcome/Centocor); BEC2 which is a murine anti-idiotype (GD3 epitope); IgG antibody (ImClone System); IMC-C225 which is a chimeric anti-EGFR IgG antibody; VITAXIN™ which is a humanized anti-αVβ3 integrin antibody (Applied Molecular Evolution/MedImmune); Campath 1H/LDP-03 which is a humanized anti CD52 IgG1 antibody (Leukosite); Smart M195 which is a humanized anti-CD33 IgG antibody (Protein Design Lab/Kanebo); RITUXAN™ which is a chimeric anti-CD20 IgG1 antibody (IDEC Pharm/Genentech, Roche/Zettyaku); LYMPHOCIDE™ which is a humanized anti-CD22 IgG antibody (Immunomedics); ICM3 which is a humanized anti-ICAM3 antibody (ICOS Pharm); IDEC-114 which is a primate anti-CD80 antibody (IDEC Pharm/Mitsubishi); ZEVALIN™ which is a radiolabelled murine anti-CD20 antibody (IDEC/Schering AG); IDEC-131 which is a humanized anti-CD40L antibody (IDEC/Eisai); IDEC-151 which is a primatized anti-CD4 antibody (IDEC); IDEC-152 which is a primatized anti-CD23 antibody (IDEC/Seikagaku); SMART anti-CD3 which is a humanized anti-CD3 IgG (Protein Design Lab); 5G1.1 which is a humanized anti-complement factor 5 (CS) antibody (Alexion Pharm); D2E7 which is a humanized anti-TNF-α antibody (CATIBASF); CDP870 which is a humanized anti-TNF-α Fab fragment (Celltech); IDEC-151 which is a primatized anti-CD4 IgG1 antibody (IDEC Pharm/SmithKline Beecham); MDX-CD4 which is a human anti-CD4 IgG antibody (Medarex/Eisai/Genmab); CDP571 which is a humanized anti-TNF-α IgG4 antibody (Celltech); LDP-02 which is a humanized anti-α4,7 antibody (LeukoSite/Genentech); OrthoClone OKT4A which is a humanized anti-CD4 IgG antibody (Ortho Biotech); ANTOVA™ which is a humanized anti-CD40L IgG antibody (Biogen); ANTEGREN™ which is a humanized anti-VLA-4 IgG antibody (Elan); and CAT-152 which is a human anti-TGF-β₂ antibody (Cambridge Ab Tech).

Other specific examples of therapeutic proteins which are contemplated for production as disclosed herein include, without limitation, factor VIII, b-domain deleted factor VIII, factor viia, factor ix, anticoagulants, hirudin, alteplase, tpa, reteplase, tpa, tpa-3 of 5 domains deleted, insulin, insulin lispro, insulin aspart, insulin glargine, long-acting insulin analogs, hgh, glucagons, tsh, follitropin-beta, fsh, gm-csf, pdgh, ifn alpa (e.g., inf-apha2), inf-beta (e.g., ifn-betal), ifn-gammalb, il-2, il-11, hbsag, ospa, murine mab directed against t-lymphocyte antigen, murine mab directed against tag-72, tumor-associated glycoprotein, fab fragments derived from chimeric mab, murine mab fragment directed against tumor-associated antigen cal25, murine mab fragment directed against human carcinoembryonic antigen, cea, murine mab fragment directed against human cardiac myosin, murine mab fragment directed against tumor surface antigen psma, murine mab fragments (fab/fab2 mix) directed against hmw-maa, murine mab fragment (fab) directed against carcinoma-associated antigen, mab fragments (fab) directed against nca 90, a surface granulocyte nonspecific cross reacting antigen, chimeric mab directed against cd20 antigen found on surface of b lymphocytes, humanized mab directed against the alpha chain of the il2 receptor, chimeric mab directed against the alpha chain of the il2 receptor, chimeric mab directed against tnf-alpha, humanized mab directed against an epitope on the surface of respiratory synctial virus, humanized mab directed against her 2, i.e., human epidermal growth factor receptor 2, human mab directed against cytokeratin tumor-associated antigen anti-ctla4, chimeric mab directed against cd 20 surface antigen of b lymphocytes domase-alpha dnase, beta glucocerebrosidase, tnf-alpha, il-2-diptheria toxin fusion protein, tnfr-lgg fragment fusion protein laronidase, dnaases, alefacept, darbepoetin alfa (colony stimulating factor), tositumomab, murine mab, alemtuzumab, rasburicase, agalsidase beta, teriparatide, parathyroid hormone derivatives, adalimumab (Iggl), anakinra, biological modifier, nesiritide, human b-type natriuretic peptide (hbnp), colony stimulating factors, pegvisomant, human growth hormone receptor antagonist, recombinant activated protein c, omalizumab, immunoglobulin e (lge) blocker and lbritumomab tiuxetan.

The invention specifically provides for the production of useful human proteins such as human proteins which have application as pharmaceutical proteins. For example, the invention provides for the production of human cytokines (such as human interferon (IFN), human erythropoietin (EPO), human growth hormone, human G-CSF, human GM-CSF), human and human antibodies and other useful human proteins. Other proteins which are desirably expressed as disclosed herein include lysozyme, β-casein, albumin, α-1 antitrypsin, antithrombin III, collagen, factors VIII, IX, X, and the like, fibrinogen, hyaluronic acid, insulin, lactoferrin, protein C, tissue-type plasminogen activator (tPA), feed additive enzymes, somatotropin, and chymotrypsin. Genetically engineered antibodies, such as immunotoxins which bind to surface antigens on human tumor cells and destroy them, can also be expressed for use as pharmaceuticals or diagnostics.

Vectors of the present invention can handle larger and thus more complex sequence payloads and can be produced rapidly in high titer. This has facilitated the creation of multiple transgenic flocks with a variety of transgenes designed for high protein expression levels in the egg white of transgenic hens. The retroviral vectors of the present invention provide a powerful tool for avian transgenesis. The simplicity of the vector along with the relativity high rates of transgenesis enable the development of many transgenic flocks for a variety of uses in the biomedical and agronomic disciplines. Retroviral vectors disclosed herein such as those based on ALV are also a powerful tool for the transgenesis of non-avian species. The vectors can efficiently integrate transgenes into human, primate and murine cells but are not able to replicate even if replication-competent vectors are utilized (Federspiel, et al. (1994) Proc Natl Acad Sci USA 91(23): 11241-11245; Rainey, et al. (2003) J Virol 77(12): 6709-19; and Hu, et al. (2007) Hum Gene Ther 18(8): 691-700). Thus, a retroviral vector system such as an ALV vector system is attractive for researchers who are hesitant to use vectors based on mammalian pathogens in mammals. Such vectors also find use in human gene therapies because, for example, ALV has little or no preference for integration into or near genes in human cells, unlike MoMLV or HIV-based vectors which tend to integrate into human genes (Mitchell, et al. (2004) PLoS Biol 2(8): E234). Thus gene therapy vectors based on vectors such as ALV have an improved safety profile as there is a lower chance for integrated vectors to interact in deleterious ways with nearby genes.

The avian retroviruses used in the present methods are typically replication deficient. Replication deficient indicates a single nucleotide sequence containing a polynucleotide encoding a retrovirus that is not able to transduce a host or target cell with the genetic information required for replication of the retrovirus. Production of replication deficient ALV is described, for example, in U.S. application Ser. No. 11/542,093, hereby incorporated herein by reference for all purposes. Briefly, for production of replication deficient ALV, fibroblast cells, e.g., DF-1 cells (see, U.S. Pat. No. 5,672,485 and Lee, et al., J Virol Methods (2008) 153(1):22-8), are transfected with a first polynucleotide sequence encoding a replication deficient retrovirus (e.g., one or more sequences encoding gag, pol and env proteins are removed) and a second polynucleotide sequence that encodes products required for replication of the replication deficient retrovirus, for example, two or more of gag, pol and env proteins. The products required are typically biomolecules that are necessary for replication or propagation of the retrovirus. For example, proteins required for replication or propagation of the retrovirus can be viral polymerase, one or more proteins contained in the viral envelope, or one or more proteins contained in the capsid.

In some embodiments the replication deficient avian retrovirus-based vectors are devoid of functional gag, pol and env genes. The replication deficient vectors can be pseudotyped with a heterologous envelope protein from another virus, e.g., vesicular stomatitis virus-G envelope protein (VSV-G) to allow for cellular adsorption of the virus. VSV-G interacts with the phospholipid components of cells, thus allowing for adsorption to any cell of vertebrate origin. Moreover, avian retroviruses pseudotyped with VSV-G continue to remain free of replication-competent virus. Other envelope proteins that find use for pseudotyping can include Avian Leukemia/Leukosis virus (ALV) envelope protein (envA).

In a particularly useful embodiment, The avian retroviruses used in the present methods are also self-inactivating (SIN). To produce a SIN retroviral vector, the 3′LTR or the 5′LTR is modified such that one or both of the 5′LTR and 3′ LTR regions are transcriptionally inactive upon integration into target cells. This leads to self-inactivation of the vector upon integration into the host genome and thus is a self-inactivating (SIN) virus. Previous non-SIN viruses have the problems that the LTR regions interfere with the activity of internal transgene promoters, altering the desired expression. As the avian retrovirus-based vectors of the present invention lack a functional endogenous vector promoter in the LTR, either a heterologous promoter located in the host's genome or an endogenous promoter located in the transgene in the vector functions to control expression of a polypeptide. Certain SIN vectors which can be useful in accordance with the invention are disclosed in herein.

SIN vectors designed and used in accordance with the invention can reduce or eliminate promoter interference of promoters of interest which are employed in transgenic avians. In a particularly useful embodiment, the promoters (i.e., promoter components) of interest preferentially express their gene product in oviduct cells or oviduct tissue, e.g., oviduct specific promoters. Examples of such promoters (e.g., promoter components) include but are not limited to, functional portions of the ovalbumin, lysozyme, conalbumin (i.e., ovotransferrin), ovomucoid, ovomucin, and/or ovoinhibitor gene expression controlling regions or promoter regions. In one embodiment, the promoter of interest is a combination or a fusion of one or more promoters or a fusion of a fragment of one or more promoters such as ovalbumin, lysozyme, conalbumin (i.e., ovotransferrin), ovomucoid, ovomucin, and/or ovoinhibitor promoters with another promoter or promoter fragment such as a viral promoter (e.g., an LTR promoter).

SIN vectors have been shown to be particularly useful with oviduct specific promoters. Without wishing to limit the invention to any particular theory or mechanism of operation it is believed that oviduct specific promoters can be particularly susceptible to influences of a retroviral LTR promoter. As a result, SIN vectors are particularly useful when employed in combination with avian oviduct specific promoters.

In one particularly useful embodiment, a SIN vector is produced in which an interfering promoter (e.g., an LTR promoter) that can at least partially inhibit transcription of a coding sequence operably linked to an oviduct specific promoter of the invention is inactivated, for example, by a deletion, insertion or transposition of all or part of the interfering promoter sequence.

In one useful embodiment, the vectors of the invention, (for example, SIN vectors, e.g., pALV-SIN vector) lack a marker such as an antibiotic resistance marker, as disclosed in US patent publication No. 2008/0064862, published Mar. 13, 2008, the disclosure of which is incorporated in its entirety herein by reference.

Without wishing to limit the invention to any particular theory or mechanism of operation it is believed that the lack of a selectable marker cassette decreases the presence of promoter elements such as enhancers which would otherwise be in cis and in close proximity to the promoter employed for exogenous protein production in avian oviduct cells (e.g., oviduct specific promoters). This close proximity may allow for interference by the transcription regulating elements of the marker gene with the promoter of interest, i.e., the promoter employed for exogenous protein production. However, the invention contemplates that marker gene coding sequences, for example, and without limitation, neomycin resistance coding sequence and beta lactamase coding sequence, may be operably linked to a promoter (i.e., second promoter) which does not interfere with the promoter employed for exogenous protein production in avian oviduct cells (i.e., first promoter). For example, it is contemplated that if the marker promoter and the promoter of interest are the same or similar promoters, interference by the selectable cassette will be minimized or eliminated. For example, a second ovalbumin promoter operably linked to a marker gene coding sequence may not interfere with a first ovalbumin promoter employed for exogenous protein production in avian oviduct cells.

The vectors (e.g., ALV-based vectors) of the present invention can provide for useful gene therapy vectors for the delivery of therapeutic polypeptides to a vertebrate host, e.g., an avian, a mammal, a human, e.g., for the treatment of disease.

The avian retroviruses used in the present methods further contain one or more polynucleotides encoding one or more therapeutic polypeptides. In some embodiments, the avian retrovirus-based vectors of the present invention contain one therapeutically relevant gene. In some embodiments the vectors of the present invention contain two therapeutically relevant genes. In some embodiments, the vectors of the present invention contain more than two therapeutically relevant genes. In some embodiments, the avian retrovirus-based vectors of the present invention contain one therapeutically relevant gene and one or more non-therapeutically relevant gene(s). In some embodiments, the vectors of the present invention contain two therapeutically relevant genes and one or more non-therapeutically relevant gene(s). In some embodiments, the vectors of the present invention contain more than two therapeutically relevant genes and one or more non-therapeutically relevant gene(s).

Avian retrovirus-based vectors of the present methods find use for the delivery of exogenous polypeptides to a variety of vertebrate subjects or patients. Examples of host vertebrates include, without limitation, avians and mammals.

In some embodiments, the vertebrate is a mammal. Exemplary mammals include humans, non-human primates (e.g., chimpanzees, macaques), agricultural mammals (e.g., equine, bovine, porcine, ovine), domesticated mammals (e.g., canine and feline) and laboratory mammals (e.g., rodents, including murine, rattus, lagomorpha, hamster). In some embodiments, the subject is a primate or domesticated mammal. In some embodiments, the subject is a human.

In a particularly useful embodiment, the vectors disclosed herein which are useful to introduce nucleotide sequences into vertebrates are produced in transient transfection methods as disclosed herein (i.e., retroviral vectors of the invention). However, it is contemplated that the retroviral vectors disclosed herein can be employed in the present invention whether of not the retroviral particles are produced as disclosed herein.

The replication deficient avian retroviral vectors of the invention find use in treating (i.e., inhibiting, ameliorating, preventing) any disease condition that has been or can be treated with gene therapy (i.e., delivery of a polynucleotide that expresses a therapeutic polypeptide). Gene therapy has been employed for the treatment or therapy of a variety of conditions, including, e.g., cancer, hormone deficiencies, enzyme deficiencies, blood factor deficiencies and diseases, autoimmune diseases, infectious diseases, as well as other diseases and conditions.

Gene therapy has been approved for a number of conditions and over 1300 clinical trials have been approved worldwide since 1989 (information regarding these numbers and trials can be found on the World Wide Web at wiley.co.uk/genmed/clinical/). The most common disease categories for which gene therapy clinical trials have been approved include cancer, vascular, monogenic, neurological and ocular diseases. (See, e.g., Edelstein, et al., Journal of Gene Medicine (2004) 6:597-602.) The vectors used in the present methods are useful for the treatment of these diseases and conditions currently in gene therapy clinical trials, as well as for the treatment of other diseases and conditions. Gene therapy protocols are known in the art, and are reviewed, for example, in LeDoux, “Gene Therapy Protocols: Volume 1: Production and In Vivo Applications of Gene Transfer Vectors (Methods in Molecular Biology),” 3^{rd} Ed., 2008, Humana Press; and LeDoux, “Gene Therapy Protocols Volume 2: Design and Characterization of Gene Transfer Vectors (Methods in Molecular Biology),” 3^{rd} Ed., 2008, Humana Press.

Just as gene therapy methods have proven successful for the treatment of many types of cancers/tumors, the avian retroviral vectors of the invention find use in the treatment of numerous cancers. (See, e.g., Cross, et al., Clinical Medicine & Research (2006) 4(3):218-227.) Types of cancers and tumors that can be treated (i.e., inhibited, reduced, prevented) by delivery of a therapeutic polypeptide using an avian retrovirus-based vector include both malignant and benign tumors, as well as solid, soft and hematological tumors. Examples of cancer diseases for which gene therapy clinical trials are currently approved include breast, ovary, cervix, glioblastoma, leptomeningeal carcinomatosis, glioma, astrocytoma, neuroblastoma, colon, colorectal, liver metastases, post-hepatitis liver cancer, prostate, renal, melanoma, head and neck, lung adenocarcinoma, lung-small cell, lung-non small cell, mesothelioma, leukemia, lymphoma, multiple myeloma, sarcoma and germ cell tumors. (See, e.g., Edelstein, et al., Journal of Gene Medicine (2004) 6:597-602.) The use of gene therapy to treat cancer is also reviewed, for example, in “Gene Therapy for Cancer (Cancer Drug Discovery and Development),” Hunt, et al., Eds., 2007, Humana Press. These cancers are suitable for treatment using the present avian retrovirus-based vectors.

Examples of cancers which are suitable for treatment by delivery of an avian retrovirus-based vector include without limitation breast, ovary, cervix, glioblastoma, leptomeningeal carcinomatosis, glioma, astrocytoma, neuroblastoma, colon, colorectal, liver metastases, post-hepatitis liver cancer, prostate, renal, melanoma, head and neck; lung cancers including, lung adenocarcinoma, lung small cell, lung non small cell; mesothelioma; hematological cancers, including leukemia, lymphoma, myeloma and multiple myeloma; sarcoma, germ cell tumors pancreatic, bladder, kidney, glioblastoma and renal cell. The avian retroviral-based vectors of the present invention can also find use for treatment or therapy of any cancers being pursued in gene therapy clinical trials, including but not limited to those listed above.

In some embodiments, cancers suitable for treatment by delivery of an avian retrovirus-based vector of the invention include those that express a tumor associated antigen and those that respond to a therapeutic polypeptide (e.g., a cytokine). (See, e.g., Haupt, et al., Experimental Biology and Medicine (2002) 227:227-237.) Retroviral expression of a therapeutic polypeptide can also be used for creating a cancer vaccine.

Examples of known tumor associated antigens (TAAs) include, e.g., melanoma associated antigens (MAGE-1, MAGE-3, TRP-2, melanosomal membrane glycoprotein gp100, gp75 and MUC-1 (mucin-1) associated with melanoma); CEA (carcinoembryonic antigen) which can be associated, e.g., with ovarian, melanoma or colon cancers; folate receptor alpha expressed by ovarian carcinoma; free human chorionic gonadotropin beta (hCGβ) subunit expressed by many different tumors, including but not limited to myeloma; HER-2/neu associated with breast cancer; encephalomyelitis antigen HuD associated with small-cell lung cancer; tyrosine hydroxylase associated with neuroblastoma; prostate-specific antigen (PSA) associated with prostate cancer; CA125 associated with ovarian cancer; and the idiotypic determinants of a B cell lymphoma can generate tumor-specific immunity (attributed to idiotype-specific humoral immune response). Moreover, antigens of human T cell leukemia virus type 1 have been shown to induce specific CTL responses and antitumor immunity against the virus-induced human adult T cell leukemia (ATL). (See, e.g., Haupt, et al., Experimental Biology and Medicine (2002) 227:227-237; Ohashi, et al., Journal of Virology (2000) 74(20):9610-9616.)

The avian retroviral-based vectors of the invention can be constructed to express polypeptides that specifically target TAAs (e.g., antibodies, ligands to the TAAs) fused to a cytotoxic polypeptide (e.g., a cytotoxin, an Fc segment to elicit antibody-dependent cytotoxicity) for the inhibition or killing of tumor cells expressing TAAs.

Successful inhibition of certain cancers has been achieved by the local expression of a cytokine. Examples of cancers that are inhibited by the proximate or local expression of a cytokine include, for example, melanoma that has been treated by expression of tumor necrosis factor or GM-CSF and glioblastoma and renal cell cancers, that have been treated with interleukin-2 expression. Further examples are provided in, e.g., Haupt, et al., Experimental Biology and Medicine (2002) 227:227-237; Dachs, et al., Oncology Research (1997) 9:313-325; and Cross, et al., Clinical Medicine & Research (2006) 3:218-227.

Tumors have also been treated by down-regulation of cellular receptor proteins. Prolonged survival of mice containing intracranial brain cancer in response to RNA interference directed towards epidermal growth factor receptor (EGFR) has been described. (See, e.g., Zhang, et al., Clinical Cancer Research (2004) 10:3667-3677.) Likewise, the avian retrovirus-based vectors of the present invention find use in delivering inhibitory nucleic acids (e.g., siRNA, miRNA, antisense polynucleotides, ribozymes) to specifically inhibit expression of proteins involved in, e.g., tumor cell growth, migration and metastasis.

In addition, melanoma, colorectal cancer and renal cell cancers have been treated with histocompatibility antigen class I B7 plus β2-microglobulin; glioblastoma and ovarian cancers have been treated with HSV thymidine kinase expression; breast cancer has been treated with multi-drug resistance 1 (MDC1) expression and head and neck squamous carcinoma has been treated with p53 expression. (See, e.g., Akporiaye, et al., Current Opinion in Molecular Therapeutics (1999) 1(4):443-453.)

The avian retroviral-based vectors can also be used to create recombinant cancer vaccines. Cancer vaccines train the patient's immune system to recognize the cancer cells (which are self) by presenting the cancer cells with highly antigenic and immunostimulatory cellular debris. Initially cancer cells are harvested from the patient (autologous cells) or from established cancer cell lines (allogeneic) and then are grown in vitro. These cells are then engineered to be more recognizable to the immune system by the addition of one or more genes, which are often cytokine genes that produce pro-inflammatory immune stimulating molecules, or highly antigenic protein genes. Specific examples of successful immunotherapy or cancer vaccine methods to trigger an immune response against tumor cells include the expression of murine α(1,3)-galactosyltransferase in allogeneic prostate cells to induce a hyperacute rejection response; the expression of CEA and MUC-1 delivered subcutaneously to produce an immune response against pancreatic cancer; the expression of GM-CSF in allogeneic prostate cells to induce an immune response against prostate tumors (GVAX vaccine); the expression of GM-CSF and CD-40L in allogeneic cells co-administered with autologous lymphoma cells to induce an immune response against lymphoma; the expression of IL-2 in autologous melanoma cells to promote an immune response against melanoma and the subcutaneous expression of CD-80 tumor antigen with IL-2 to induce an immune response against renal cell carcinoma. See, Cross, et al., (2006), supra.

Malignant hematologic disorders can be treated with chimeric antigen receptors (CARs). A chimeric antigen receptor is a single chain antibody with specificity for an antigen expressed on a tumor cell that is linked to an internal kinase F domain which mediates cell activation when the antibody is engaged by the target antigen. CARs targeting CD19 expression on human B-cell malignancies have been used for B-cell tumor treatment. (See, e.g., Bae, et al., Clinical Cancer Research (2005) 11(4):1629-1638.)

The avian retroviral-based vectors of the invention also find use in treating various enzyme deficiency and hormone deficiency conditions, for example, by delivery of functional sequences of the deficient enzymes and hormones and by the genomic integration of polynucleotides encoding functional sequences of the deficient enzymes and hormones.

Examples of hormone and enzyme deficiencies (sometimes referred to as monogenic diseases) for which gene therapy clinical trials have been pursued include Hurler's syndrome, Hunter's syndrome, Gaucher's disease, purine nucleoside phosphorylase deficiency, ornithine transcarbamylase deficiency and Fabry disease. The avian retroviral-based vectors of the present invention find use for treatment of any monogenic diseases/hormone and enzyme deficiencies being pursued in gene therapy clinical trials, including but not limited to those listed above.

Further examples of enzyme deficiency diseases that can be treated using the replication incompetent and self-inactivating avian retroviral-based vectors of the present invention include: Lesch-Nyhan which has been treated with expression of hypoxanthine-guanine phosphoribosyl transferase; phenylketonuria (PKU) which has been treated with expression of phenylalanine hydroxylase; emphysema which has been treated with expression of alpha-1-antitrypsin (AAT); Gaucher's disease has been treated with glucocerebrosidase (Cerezyme); Wolman's Disease (WD) and cholesteryl ester storage disease (CESD) have been treated with lysosomal acid lipase (cholesterase); galactosialidosis (GS) has been treated with β-Galactosidase and Neuraminidase; sialidosis has been treated with neuraminidase; CNS (central nervous system) disease have been treated with galactosylceramidase (GALC); Fabry Disease has been treated with Agalsidase alpha (Replagal), Agalsidase beta (Fabrazyme) or alpha galactosidase A; Pompe disease has been treated with alpha-glucosidase (MYOZYME); Niemann-Pick Disease type AB has been treated with Acid Sphingomyelinase (rhASM); and Globoid cell leukodystrophy (GLD, Krabbe disease or CNS disease) has been treat with galactosylceramidase (GALC).

Several mucopolysaccharidosis (MPS) diseases (also known as lysosomal storage diseases) can be successfully treated by gene therapy methods as well. The MPS diseases for which the avian retroviral-based vectors of the present invention can find use include MPS I (Mucopolysaccharidosis Type I or Hurler Syndrome) has been treated with alpha-L-iduronidase (ALDURAZYME); MPS II (mucopolysaccharidosis type II or Hunter Syndrome) has been treated with idursulfase (Elaprase); MPS IIIA (Mucopolysaccharidosis Type IIIA or Sanfilippo Syndrome) has been treated with heparin sulfamidase; MPS IIIB (Mucopolysaccharidosis Type IIIB or Sanfilippo syndrome type IIIB or Sanfilippo Syndrome) has been treated with N-acetylglucosaminidase; MPS IIIC (Mucopolysaccharidosis Type IIIC or Sanfilippo Syndrome) has been treated with alpha-glucosaminide N-acetyltransferase; MPS IIID (Mucopolysaccharidosis Type IIID or Sanfilippo Syndrome) has been treated with N-acetylglucosamine-6-sulfate sulfatase; MPS IV type A (Morquio syndrome) has been treated with N-acetylgalactosamine 6-sulfatase (GALNS or galactose 6-sulfatase); MPS IV type B (Morquio syndrome) has been treated with beta-galactosidase; MPS VI (Mucopolysaccharidosis Type VI or Maroteaux-Lamy syndrome) has been treated with galsulfase (NAGLAZYME) as well as arylsulfatase B, recombinant human arylsulfatase B, rhASB, BM 102 or N-acetylgalactosamine-4-sulfatase) and MPS VII (mucopolysaccharide disease also known as Sly syndrome) has been treated with glucoronidase. Gene therapy clinical trials for MPS I, MPS II, MPS IIIA, MPS VI and MPS VII have been pursued. (See, e.g., Sly, et al., PNAS (2002) 99(9):5760-5762 and the World Wide Web at wiley.co.uk/genmed/clinical/.) The avian retroviral-based vectors of the present invention find use for treatment of any MPS/lysosomal storage disease including but not limited to those listed above.

Examples of hormone deficiency diseases that can be treated using replication incompetent and self-inactivating avian retroviral-based vectors include heat stress has been treated with expression of plasmid growth hormone-releasing hormone treatment; growth hormone deficiency treatment with expression of growth hormone (GH); leptin expression for the treatment of obesity; and insulin for the treatment of diabetes. Other indications that can be treated with hormone gene therapy include fractures. For example, gene therapy clinical trials employing parathyroid hormone to treat tibia fractures have been pursued. (See, e.g., Edelstein, et al., Journal of Gene Medicine (2004) 6:597-602 and the World Wide Web at wiley.co.uk/genmed/clinical/.) The avian retroviral-based vectors of the present invention find use for treatment of any hormone disease including but not limited to those listed above. The avian retroviral-based vectors of the present invention find use for treatment of any indication for which hormone gene therapy can find use including but not limited to those listed above.

The avian retroviral-based vectors of the invention find further use in the treatment and therapy of blood factor deficiencies and blood disorders which have been successfully treated using gene therapy. (See, e.g., Nienhuis, Blood (2008) 111(9):4431-4444.) Examples of blood factor deficiencies and blood disorders (some of which are referred to as monogenic diseases) for which gene therapy clinical trials are currently being pursued include Haemophilia A and B, Fanconi's anaemia, Leukocyte adherence deficiency and chronic granulomatous disease. (See, e.g., Edelstein, et al., Journal of Gene Medicine (2004) 6:597-602.) The vectors of the present invention find use for treatment of any blood factor deficiencies and blood disorders being pursued in gene therapy clinical trials, including but not limited to those listed above.

Further examples of blood factor deficiencies and blood disorders that can be successfully treated using the avian retrovirus-based vectors of the invention include Fanconi Anemia which has been treated with replacement of one of the seven Fanconi anemia proteins (FANCA, FANCB/D1, FANCC, FANCE, FANCF or FANCG); blood coagulation Factor X deficiency which has been treated with expression of Factor X; Hemophilia A which has been treated with Factor VIII expression; Hemophilia B which has been treated with expression of Factor IX; chronic granulomatous disease (CGD) has been treated with expression of one or more proto-oncogenes including MDS1-EV11, PRDM16 and SETBP1; hemoglobin disorders which have been treated with expressions of globins; sickle cell anemia has been treated with expression of globins; β-thalassemia has been treated with expression of globins; and prevention of clot formation can be achieved with an antibody against anti-glycoprotein IIb/IIIa receptor on platelets. The avian retroviral-based vectors of the present invention find use for the treatment of any blood factor deficiencies and blood disorders including but not limited to those listed above.

The avian retroviral-based vectors of the invention further find use in the expression of an immunostimulatory polypeptide for the treatment of an immunodeficiency disease or an immunosuppressing polypeptide for the treatment of a pathological immune response, e.g., an autoimmune disease.

Examples of immunodeficiency disorders and diseases (sometimes referred to as monogenic diseases) for which gene therapy clinical trials are currently being pursued include SCID (treated with adenosine deaminase (ADA)), chronic granulomatous disease (treated with autologous hematopoietic stem cells transduced with MT-gp91). (See, e.g., Edelstein, et al., Journal of Gene Medicine (2004) 6:597-602.) The vectors of the present invention find use for treatment of any immunodeficiency disorders being pursued in gene therapy clinical trials, including but not limited to those listed above. Further examples of immunodeficiency disorders and diseases which can be treated using the present avian retroviral-based vectors include, for example, AIDS which has been treated with HSV thymidine kinase expression; Severe Combined Immunodeficiencies (SCIDS; ADA-SCIDS, adenosine deamininase SCIDS) which have been treated with adenosine deaminase expression; X-SCID has been treated with expression of IL-2Rγ (gamma-chain of interleukin-2 receptor) and γc; and purine nucleoside phosphorylase deficiency (a disease resulting in immunodeficiency) has been treated with expression of purine nucleoside phosphorylase (PNP). The avian retroviral-based vectors of the present invention find use for the treatment of any immunodeficiency disorders and diseases (including autoimmune diseases) including but not limited to those listed above.

Pathological immune disorders that can be inhibited using the avian retroviral vectors of the invention include graft versus host disease (GVHD) and autoimmune diseases. So called “suicide gene therapy” can be employed for graft versus host disease (GVHD) therapy. The avian retroviruses express suicide genes (e.g., pro-apoptotic polypeptides) in immune cells mediating the pathological GVHD response, thereby making the immune cells susceptible to drug-induced death. Autoimmune diseases can be treated by the expression, e.g., in muscle, of autoantigens targeted in the autoimmune disease. For example, the expression in muscle of myelin basic protein can be used to suppress a pathological immune response in multiple sclerosis; the expression of an insulin protein can be used to suppress a pathological immune response in insulin dependent diabetes; and the expression of interphotoreceptor retinoid-binding protein (IRBP) or S-antigen can be used to suppress a pathological immune response in autoimmune uveitis. See, e.g., U.S. Patent Publication No. 2003/0148983. The avian retroviral-based vectors of the present invention find use for the treatment of any pathological immune disorders including but not limited to those listed above.

Examples of inflammatory conditions which can be treated by delivering a therapeutic polypeptide using the avian retroviral-based vectors include inflammatory bowel disease (e.g., inflammatory disease of the rectum has been treated with interleukin-4 (IL-4) or interleukin-10 (IL-10)), rheumatoid arthritis (treatment with tumor necrosis factor receptor-Fc immunoglobulin (TNFR:Fc) fusion or interleukin-1 receptor antagonist protein) and carpal tunnel syndrome.

The avian retroviral-based vectors of the invention also find use in the treatment of a variety of infectious diseases that have been successfully treated using gene therapy. (See, e.g., Brunnell, et al., Clinical Microbiology Reviews (1998) 11(1):42-56.)

Examples of infectious diseases for which clinical trials are currently being pursued include HIV/AIDS, tetanus, CMV infection and adenovirus infection. (See, e.g., Edelstein, et al., Journal of Gene Medicine (2004) 6:597-602 and the World Wide Wed at wiley.co.uk/genmed/clinical/). The vectors of the present invention find use for treatment of any infectious diseases being pursued in gene therapy clinical trials, including but not limited to those listed above.

Viral infectious diseases that can be treated using the avian retrovirus-based vectors include, for example, chronic viral hepatitis which has been treated with interferon alpha. Other examples include Human Immunodeficiency Virus (HIV-1) infection which has been treated with antisense therapy to the viral genes tat, rev, vpu and gag; ribozyme therapy that targets viral gag; expression of transdominant negative mutant Rev or mutant RevM10 or infection of cells to express the HIV-1IIIB gene to induce cellular and humoral immune responses.

Human T-Cell Lymphotrophic Virus (HTLV-1) infection has been treated with transdominant mutant Rex, a protein essential for replication. Influenza virus infection has been treated with gene vaccines containing conserved influenza proteins. Human Papillomavirus (HPV) infection which has been treated with antisense oligonucleotides or antisense RNA, or ribozymes that specifically cleave PPV E7 RNA.

Hepatitis B virus (HBV) infection has been treated with interferon alpha or antisense oligonucleotide and deoxyoligonucleotide therapy directed to the gene encoding surface antigen (pre-S). Hepatitis C virus (HCV) infection has been treated with interferon alpha; antisense oligonucleotide therapy treatment directed different stem loop structures in the 5′-noncoding region (NCR) of the HCV RNA or antisense oligonucleotide therapy treatment directed against a nucleotide stretch including the start codon of the polyprotein precursor of HCV

The Herpes Simplex Virus (family includes HSV-1, HSV-2, varicella-zoster virus, Epstein-Barr virus (EBV) and cytomegalovirus). HSV-1 has been treated with antisense oligonucleotides or transdominant negative protein variants of the HSV-1 viral proteins. Epstein-Barr Virus (EBV) has been treated with antisense oligonucleotide strategy focuses on blocking EBNA1, a protein required for viral replication. Cytomegalovirus (CMV) has been treated with antisense oligonucleotides complementary to the IE1, IE2 UL36 and UL37 viral regions. The avian retroviral-based vectors of the present invention can be used to treat viral infectious diseases including but not limited to those listed above.

Bacterial infectious diseases which can be treated with the avian retroviral-based vectors include Mycobacterium tuberculosis which has been treated with genetic vaccines expressing Hsp65 or antigen 85. Expression of antigen 85A (Ag85A) is in clinical trials for the prevention and treatment of tuberculosis. The avian retroviral-based vectors of the present invention can be used to treat bacterial infectious diseases including but not limited to those listed above. For a review of gene therapy methods pertaining to infectious diseases see, e.g., Bunnell, et al., Clinical Microbiology Reviews (1995) 11(1):42-56.

Examples of vascular and cardiovascular diseases which can be treated by delivering a therapeutic polypeptide using the avian retroviral-based vectors include peripheral arterial disease (by expression of fibroblast growth factor (FGF) or vascular endothelial growth factor (VEGF)), coronary artery disease (by expression of fibroblast growth factor (FGF) or vascular endothelial growth factor (VEGF)), coronary heart disease (by expression of angiogenic factors), venous ulcers (by expression of platelet-derived growth factor (PDGF)), vascular complications of diabetes (by expression of platelet-derived growth factor (PDGF)) and familial hypercholesterolemia (treatment with LDL receptor). (See, e.g., Kim, et al., Experimental and Molecular Medicine (2004) 36(4):336-344; Lathi, et al., Anesthesia & Analgesia (2001) 92:19-25; Edelstein, et al., Journal of Gene Medicine (2004) 6:597-602.)

Examples of neurological diseases which can be treated by delivering a therapeutic polypeptide using the avian retroviral-based vectors include Huntington's chorea (by expression of ciliary neurotrophic factor (CNTF)), Parkinson's disease (by expression of subthalamic glutamic acid decarboxylase (GAD) gene transfer or expression of aromatic L-amino acid decarboxylase), Alzheimer's disease (by expression of nerve growth factor (NGF)), diabetic neuropathy (by expression of VEGF or VEGF-2), Canavan disease (by expression of aspartoacylase (ASPA)), Myesthenia Gravis (by expression of a oligodeoxynucleotide inhibitory to the acetylcholinesterase (ACHE) gene) and amyotrophic lateral sclerosis (by expression of Ciliary neurotrophic factor (CNTF) or EAAT2). The avian retrovirus-based vectors also find use in the treatment of Duchenne Muscular Dystrophy (ALS; by expression of inhibitory oligonucleotides that induce exon skipping in exon 51 of the DMD gene or minidystrophin) and Junctional epidermolysis bullosa (by expression of laminin 5-beta3), both of which are monogenic diseases. (See, e.g., Edelstein, et al., Journal of Gene Medicine (2004) 6:597-602, as well on the World Wide Web at wiley.co.uk/genmed/clinical/.)

Examples of ocular diseases which can be treated by delivering a therapeutic polypeptide using the avian retroviral-based vectors of the invention include retinal degeneration (by expression of RPE65) gyrate atrophy (by expression of ornithine aminotransferase (OAT)), superficial corneal opacity (by expression of dominant negative cyclin G1 (dnG1 cyclin)), retinitis pigmentosa (by expression of ciliary neurotrophic factor (CNTF)), atrophic macular degeneration (by expression of ciliary neurotrophic factor (CNTF) and glaucoma (by expression of p21 WAF-1/Cip1). (See, e.g., Edelstein, et al., Journal of Gene Medicine (2004) 6:597-602, as well on the World Wide Web at wiley.co.uk/genmed/clinical/.)

Examples of other diseases which may be treated using avian retroviral-based vectors of the invention include chronic renal disease (also known as chronic kidney disease; by expression of erythropoietin (EPO)), fractures (tibia and hip fractures treated with parathyroid hormone) and erectile dysfunction (treatment with Maxi-K Channel hSlo).

Other examples of diseases for which gene therapy has proven useful, and which can be successfully treated using the avian retroviral-based vectors of the invention include Cystic fibrosis which can be treated by expressing cystic fibrosis transmembrane receptor (CFTR; also known as cystic fibrosis transmembrane conductance regulator); erectile dysfunction which can be treated by expressing hMaxi-K (stimulates potassium ion transfer); neonatal autosomal recessive pulmonary disease which can be treated by expression of surfactant protein B (SF-B). The avian retroviral-based vectors of the present invention find use for treatment of any of the diseases listed above.

For a review of gene therapy clinical trials currently approved, see, e.g., Edelstein, et al., Journal of Gene Medicine (2004) 6:597-602, as well as information located on the World Wide Web at wiley.co.uk/genmed/clinical/; see also Melman, et al., IMAJ (2007) 9:143-146.). The avian retroviral-based vectors of the present invention find use for treatment of any of the diseases listed above. The avian retroviral-based vectors of the present invention find use for treatment of any other diseases or deficiencies being pursued in gene therapy clinical trials.

Any gene or nucleotide sequence that codes for a therapeutically relevant polypeptide can be delivered using the avian retroviral vectors of the present invention. The avian retroviral-based vectors find use in delivering polynucleotide encoding any kind of therapeutic polypeptide, including without limitation, immunogenic polypeptides, toxins, immunotoxins, antigens, transmembrane proteins, cytokines, enzymes, enzyme inhibitors, chemokines, tyrosine kinase receptors, cell cycle genes, cytostatic agents, metabolic enzymes, polypeptides having an anti-tumor effect, antibodies, tumor suppressors, anti-angiogenic factors, apoptosis inhibitors, anti-infectious cellular proteins, intrabodies (single-chain antibodies expressed intracellularly), chimeric antigen receptors, suicide genes, apoptosis initiators, cellular division inhibitors or signal transduction inhibitors. Additionally genes encoding polypeptides capable of modulating and/or regulating expression of corresponding genes, polypeptides capable of inhibiting a bacterial, parasitic or viral infection or its development, (e.g., antigenic polypeptides and antigenic epitopes), as well as transdorninant negative proteins are therapeutic polypeptides that can be delivered using the avian retrovirus-based vectors of the invention.

While the therapeutic polypeptides subject for delivery by the avian retroviruses of the invention have been generally categorized herein for clarity, this by no means implies any single function or category for any listed proteins. As any individual of skill in the art will realize, proteins exhibit a wide variety of function in individuals. The present categorizations are in no way intended to limit any of the listed proteins to any specific use. The therapeutic polypeptides contemplated for delivery by the avian retroviral vectors can be used for therapy or treatment in any pharmaceutically relevant context for any appropriate disease or condition. Further therapeutic polypeptides are described in U.S. application Ser. No. 11/542,093, hereby incorporated herein by reference for all purposes.

The avian retroviral-based vectors of the invention can be used to deliver to a vertebrate host multimeric proteins including immunoglobulins, include antibodies, and antigen binding fragments thereof. Thus, in one embodiment of the present invention, the multimeric protein is an immunoglobulin, wherein the first and second heterologous polypeptides are immunoglobulin heavy and light chains respectively. In some embodiments, the antibody is a single-chain of the variable regions (scFv).

In some embodiments, an immunoglobulin polypeptide encoded by the transcriptional unit of at least one expression vector may be an immunoglobulin heavy chain polypeptide comprising a variable region or a variant thereof, and may further comprise a D region, a J region, a C region, or a combination thereof. An immunoglobulin polypeptide produced as disclosed herein may also be an immunoglobulin light chain polypeptide comprising a variable region or a variant thereof, and may further comprise a J region and a C region. The present invention also contemplates multiple immunoglobulin regions that are derived from the same animal species, or a mixture of species including, but not only, human, mouse, rat, rabbit and chicken. In certain embodiments, the antibodies are human or humanized.

In other embodiments, the immunoglobulin polypeptide produced as disclosed herein comprises an immunoglobulin heavy chain variable region, an immunoglobulin light chain variable region, and a linker peptide thereby forming a single-chain antibody capable of selectively binding an antigen.

Generally, the antibodies can be directed against any desired antigen, for example, tumor associated antigens, immune cell antigens, cell adhesion antigens, cytokines, and other targets. The delivered antibodies can be therapeutic polypeptides in themselves or can be used as a fusion protein to deliver a therapeutic polypeptide to a desired target. Monoclonal antibodies against the targets listed herein are known in the art, and therefore their encoding sequences can be packaged into the avian retroviral vectors of the invention.

Antibodies expressed by the avian retroviral-based vectors of the invention can be directed against tumor associate antigens (TAA) expressed by a variety of tumor types and include the following examples. See, e.g., Cross, et al., Clinical Medicine & Research (2006) 4(3): 218-227; Stoff-Khalili, et al., Cancer Gene Therapy (2006) 13(7):633-647; Perricone, et al., Molecular Therapy (2000) (1)(3):275-284.

Melanoma associated antigens include MAGE antigens, MAGE-1, MAGE-3, MART-1, tyrosine-related protein-1 (TRP-1) and tyrosine related protein-2 (TRP-2)). Melanocyte differentiation antigens include for example MDA, MDA TRP-1, MDA-7 (IL-24), MDA gp100 (melanosomal membrane glycoprotein gp100), melanocyte differentiation marker gp75, tumor-associated glycoprotein and cytokeratin.

Bladder and urethral tumor associated antigens include peptides derived from Uroplakin(UP), for example Uroplakin Ia, Uroplakin (UP) Ib, Uroplakin(UP) II and Uroplakin(UP) III). Prostate tumor associated antigens include Prostate specific antigen (PSA), prostate acid phosphatase (PAP), and prostate specific membrane antigen (PSMA). Ovarian carcinoma associated antigens include folate receptor, folate receptor alpha and CA125. Breast cancer associated antigens include BA-46 (Lactadherin), BRCA-1, BRCA-2, HER2/neu, CRIPTO-1 (CR-1), Uroplakin Ia, Uroplakin(UP) Ib, Uroplakin(UP) II and Uroplakin(UP) III).

Teratoma associated antigens include Teratocarcinoma-derived growth factor (TDGF). Gastric tumor associated antigens include Mucin (MUC-1 and cytokeratin. Lung tumor associated antigens include encephalomyelitis antigen HuD. Neuroblastoma associated antigens include tyrosine hydrolase. Lymphoma associated antigens include the idiotypic determinants of B cell lymphoma and CD20. Leukemia associated antigens include human T cell leukemia virus type 1 antigens. Colorectal tumor associated antigens include tumor-associated glycoprotein-72 (TAG-72). Tumor antigens associated with a variety of tumors include carcinoembryonic antigen (CEA), human chorionic gonadotropin beta (hCGβ) subunit, epidermal growth factor receptor (EGFR), cytokeratin and β2-microglobulin.

Antibodies expressed by the avian retroviral-based vectors of the invention also can be directed against immune cell antigens or cell surface proteins.

Exemplary immune cell antigens that serve as therapeutic targets include, CD3, CD4, CD8, CD20, CD25, CD33, CD52, CD80, CD40L, Fas, FasL, B7, CD28, CTLA4, among others known in the art.

Antibodies expressed by the avian retroviral-based vectors of the invention also can be directed against cell adhesion and cell surface antigens.

Exemplary cell adhesion antigens include integrins (e.g., αVβ3, α4β7, VLA-4), selectins and ICAMs (e.g., ICAM3), among others known in the art.

Delivery of antibodies against other targets, including cytokines (e.g., TNFα, TGFβ), cytokine receptors (e.g., IL-2 receptor alpha or gamma chains), anti-glycoprotein IIb/IIIa receptor and complement proteins (e.g., complement factor 5) also find use.

The avian retroviral-based vectors can be used to deliver one or more cytokines. Exemplary cytokines include Th1 cytokines (e.g., IL-2, IFN-γ, TNFβ, IL-12, IL-15), Th2 cytokines (e.g., IL-4, IL-5, IL-6, IL-10), immunosuppressive cytokines (e.g., TGFβ, IL-10), IL-12 family cytokines (IL-12, IL-23, IL-27), pro-inflammatory cytokines (e.g., IL-1, IL-12, IL-15, IL-18, TNFα, IFN-γ), chemokines (e.g., interferon-induced protein 10 (IP-10), monocyte chemotactic protein-I (MCP-1), MCP-2, MCP-3, MCP-4, macrophage inflammatory protein 1 (MIP1), MIP2, MIP3, RANTES (CC chemokine ligand 5), macrophage-derived chemokine (MDC), stromal cell-derived factor 1 (SDF-I), monokine induced by IFN-gamma (MIG)), hematopoietic cytokines (e.g., GM-CSF, G-CSF), interferons (e.g., IFN-α, IFN-β, IFN-γ), growth factors (e.g., PDGF, EGF, FGF, NGF, IGFs, TGFs, BMPs 1 to 12, and CNTF), pro-apoptotic polypeptides (e.g., “suicide genes”) and checkpoint proteins (e.g., MDC1, p53).

The cytokines can be delivered using the avian retroviral vectors directly to the vertebrate host or ex vivo to cells that will express and secrete the cytokine once delivered to the host. In one embodiment, the avian retroviral vectors are used to deliver the polynucleotide encoding the cytokine to a tumor infiltrating lymphocyte (TIL). TIL therapy is well known and established therapy that has found use in the treatment of a variety of cancers, including renal cell carcinoma and melanoma. TIL cells inherently target tumors and thus provide a method for targeting of proteins to tumors. In conjunction with TIL therapy, TIL cells can be induced to express therapeutic protein(s) of interest has been described as being combinable with gene therapy methods (see, e.g. U.S. Pat. No. 5,656,465). Due to the inherent targeting of the TIL cells to the tumor site, a therapeutic protein being expressed can be targeted to the tumor as well.

Examples of proteins that find use in TIL therapy include but are not limited to TNF, cytokines, interleukins (including IL-2, IL-4, IL-10 and IL-12), interferons (include IFN-γ), granulocyte macrophage colony stimulating factor (GM-CSF) and co-stimulatory factor (include B7). In some embodiments a single therapeutic protein is expressed. In some embodiment two therapeutic proteins are expressed. In some embodiments two or more therapeutic proteins are expressed.

The avian retroviral-based vectors of the invention can be used to deliver one or more hormones. Examples of hormones that can be delivered using the present avian retrovirus-based vectors include glycoproteins (including follicle stimulating hormone (FSH), follitropin-alpha, follitropin-beta, alteplas, alpha-fetoprotein e), growth hormones (including growth hormone (GH), human growth hormone (HGH), human growth hormone receptor antagonist, plasmid growth hormone-releasing hormone, pegvisomant, somatotropin (bovine growth hormone)), peptide hormones (including insulin, insulin lispro, insulin aspart, insulin glargine, long-acting insulin analogs, luteotropic hormone (LTH), prolactin, prolactin fragment, leptin, thyroid stimulating hormone (TSH), glucagons, thyroid hormone), hormones involved in blood coagulation/anticoagulation pathways (including hirudin, reteplase, tissue plasminogen activator (tPA), tPA with 3 of 5 domains deleted) and parathyroid hormones (including teriparatide, parathyroid hormone, parathyroid hormone derivatives).

The avian retroviral-based vectors of the invention can be used to deliver one or more enzymes. Examples of enzymes that can be delivered using the present avian retrovirus-based vectors include nucleotide and nucleic acid modifying enzymes (e.g., hypoxanthine-guanine phosphoribosyl transferase; telomerase catalytic protein; telomerase reverse transcriptase, DNase (deoxyribonuclease), Dornase-alpha (Dornase-alfa), RNase A, adenosine deaminase and purine nucleoside phosphorylase). Metalloproteinases including METH-1 (ADAMTS-1; fragments thereof) and METH-2 (ADAMTS-8; fragments thereof) can be delivered. Feed additive enzymes including xylanase and β-glucanase can also be delivered.

The avian retroviral-based vectors can be used to enzymes that affect proteins. Protein modifying enzymes including cysteine aspartic acid proteases, e.g., caspase 8 and papain can be delivered. Amino acid modifying enzymes, e.g., phenylalanine hydroxylase, TrpRS (human tryptophanyl-tRNA synthetases and fragments thereof), TyrRS (human tyrosyl-tRNA synthetases and fragments thereof) can be delivered. Proteolytic enzymes including alpha-1-antitrypsin (AAT, A1A), chymotrypsin, trypsin, proteinase K and carboxypeptidase can be delivered.

Hydrolytic enzymes can also be delivered, including lysozyme, lysosomal hydrolase alpha-galactosidase A, agalsidase beta (FABRAZYME), beta-galactosidase, lysosomal acid lipase (cholesterase), neuraminidase, heparin sulfamidase, alpha-glucosidase (alglucosidase alfa, MYOZYME), N-acetylglucosaminidase, alpha-glucosaminide N-acetyltransferase, N-acetylglucosamine-6-sulfate sulfatase, N-acetylgalactosamine 6-sulfatase (GALNS or galactose 6-sulfatase), galsulfase (NAGLAZYME), N-acetylgalactosamine-4-sulfatase, galactosylceramidase (GALC), glucoronidase, ALDURAZYME (laronidase), alpha-L-iduronidase(Aldurazyme), idursulfase (Elaprase), acid sphingomyelinase (rhASM), arylsulfatase A and arylsulfatase B. Glycosidases including beta glucocerebrosidase can be delivered. Glycolysis enzymes including triosephosphate isomerase can be delivered. Glycosyltransferases including murine α(1,3)-galactosyltransferase, fucosyltransferases, and sialyltransferases can be delivered. Urate oxidases including rasburicase can also be delivered.

The avian retroviral-based vectors of the invention can be used to deliver one or more genes useful in the treatment and therapy of blood factor deficiencies and blood disorders. Examples of genes useful in the treatment and therapy of blood factor deficiencies and blood disorders include but are not limited to the seven Fanconi anemia proteins including FANCA, FANCB/D1, FANCC, FANCE, FANCF and FANCG.

Examples also include coagulation regulating proteins including anticoagulants, antithrombin III, thrombin (activated Factor II or Ia), tissue-type plasminogen activator (tPA), Factor VIIa Factor VIII, Factor IX, Factor X, Factor VIII (b-domain deleted) and anti-glycoprotein IIb/IIIa receptor can be delivered. Platelet aggregation inhibitors including REOPRO (abciximab) can be delivered. Chemokines including MIP-1 alpha (macrophage inflammatory protein-1 alpha) and MIP-3 alpha (macrophage inflammatory protein 3 alpha) can be delivered. Antiangiogenic proteins including thrombospondin can be delivered. Further, Erythropoiesis proteins including erythropoietin (EPO), human erythropoietin, darbepoetin alfa and erythropoietin derivatives can also be delivered.

Proto-oncogenes including MDS1-EVI1, PRDM16 and SETBP1 can be delivered. Globins including beta globing and alpha globin can be delivered. Chimeric antigen receptors including CARs targeting CD19 (CARs are single chain antibodies with specificity for an antigen expressed on a tumor cell that is linked to an internal kinase F domain which mediates cell activation when antibody is engaged by the target antigen) can be delivered.

The avian retroviral-based vectors of the invention can be used to deliver one or more viral proteins or therapeutic toxins. Examples of viral protein genes useful in gene therapy methods and that can be delivered include Human Papilloma Virus (HPV) genes (e.g., E6, E7, L1, L2; antisense or ribozymes that specifically cleave PPV E7 gene RNA), Human Immunodeficiency Virus (HIV) genes (including tat, rev, vpu, gag, Rev, RevM10, HIV-1IIIB; HTLV-1 mutant Rex gene, including antisense and ribozymes therapy), Herpes Simplex Virus genes (including HSV-1 viral genes, HSV thymidine kinase), Epstein-Barr Virus (EBV) genes (EBNA1, a protein required for viral replication, including antisense therapy), Cytomegalovirus (CMV) genes (IE1, IE2 UL36 and UL37 viral region, including antisense therapy), Hepatitis B Virus genes (including surface antigen HBSAG), and Hepatitis C Virus genes.

Examples of protein toxins useful in the gene therapy methods and deliverable using the present avian retroviral-based vectors include protein toxins (including ricin and abrin), bacterial polypeptide chains (including polypeptides from diphtheria and IL-2-diptheria toxin fusion protein, Pseudomonas exotoxin), Mycobacterium tuberculosis genes (including Hsp65 and antigen 85) and Borrelia burgdorferi genes (including Outer Surface Protein A (OspA)).

Examples of other proteins deliverable using the avian retroviral-based vectors of the invention include cellular receptors (e.g., cystic fibrosis transmembrane receptor (CFTR), LDL receptor), S-antigen, interphotoreceptor retinoid-binding protein (IRBP), hMaxi-K and surfactant proteins (including surfactant protein B (SF-B)), as well as others described herein and known in the art.

The avian retroviral-based vectors of the invention are also contemplate f for enhancing the immunostimulatory effect of an antigen encoded by nucleic acid contained in a nucleic acid construct. It has been described that the presence of unmethylated CpG motifs in DNA vaccines is essential for the induction of immune responses against the antigen (Sato, et al., Science (1996) 273:352-354; Klinman, et al., The Journal of Immunology (1997) 158(8):3635-3639). The DNA vaccine can provide its own adjuvant in the form of CpG DNA. Changes in immunostimulatory effects can be modulated via the CpG motifs. For immunostimulatory increase, the neutralizing CpG (CpG-N) motifs in the construct can be removed and the stimulatory CpG (CpG-S) motifs in the construct can be inserted, thereby producing a nucleic acid construct having enhanced immunostimulatory efficacy. For gene therapy purposes, neutralizing CpG-N are desirable and CpG-S are undesirable. In the case of DNA vaccines, removal of CpG-N motifs and addition of CpG-S motifs allows induction of a more potent and appropriately directed immune response. These methods are described in Krieg et al., US 2004/0186067. The avian retroviral-based vectors of the present invention containing CpG motifs elements can be used as vaccines.

Specific immunostimulatory effects have been described for certain nucleotide sequences. Sato et al., Science (1996) 273:352-354 describes the effects of vaccination with double stranded DNA having certain CpG containing sequences on the production of interferon-gamma, interferon-beta, and interleukin-12.

Immunostimulatory effects with gene therapy methods have also been shown for enhanced immune responses to the melanoma associated antigens, including for example enhanced immune responses to gp100, MART-1, tyrosine-related protein-1 (TRP-1) and tyrosine related protein-2 (TRP-2). (See, e.g., Perricone, et al., Nature, 2000, 1(3):275-284.)

Cellular immune response elements can also be used to regulate the immune system and are well to know to those of skill in the art. Cellular immune response elements can include, for example, IFN-γ and IL-2 which can regulate Th1 responses; IL-4, IL-5, IL-10, IL-13 which can regulate Th2 responses and TGF-β with CD4⁺CD25⁺ regulatory cells which can regulate Th3 responses. Sequences from the above listed immune system regulatory proteins can be included in vectors to generate vectors useful as vaccines. (See, e.g., Yoo, WO 2008/097927.) The avian retroviral-based vectors of the present invention expressing or co-expressing a regulatory cytokine with an immunogenic polypeptide can be used as vaccines. The avian retroviral-based vectors of the present invention can contain cellular immune response elements. The vectors can further include sequences encoding cellular immune response elements, e.g., cytokines that promote a Th2 immune cell profile. The avian retroviral-based vectors of the present invention containing cellular immune response elements can be used as vaccines.

In the case of DNA vaccines, removal of CpG-N motifs and addition of CpG-S motifs should allow induction of a more potent and appropriately directed immune response. The opposite approach with gene therapy vectors, namely the removal of CpG-S motifs and addition of CpG-N motifs, allows longer lasting therapeutic effects by abrogating immune responses against the expressed protein. These methods are described in Krieg et al., US 2004/0186067. The avian retroviral-based vectors of the present invention containing CpG motifs elements can be used as vaccines.

Whether a polynucleotide is immunostimulatory or immunosuppressive can also depend on the route of delivery. The avian retroviral-based vectors of the invention (e.g., particles) can be introduced by oral and nasal administration. Oral, nasal and intramuscular autoantigen administration has been shown to suppress pathological immune responses in autoimmune diseases including multiple sclerosis, autoimmune uveitis, thyroiditis, myasthenia gravis, rheumatoid arthritis and insulin-dependent diabetes mellitus. Therapeutic polypeptides can be delivered by the avian retroviral-based vectors of the present invention for the purpose of suppressing a pathological immune response. As discussed above, the avian retroviral-based vectors can deliver one or more autoantigens targeted in an autoimmune response. Delivery can be by any route that promotes immune cell anergy or immunosuppression, e.g., oral, intranasal, intramuscular, intravenous.

The avian retroviral-based vectors of the present invention can be administered via a any routes appropriate for treatment of the condition to be treated. The routes of delivery can include systemic administration and administration in situ. The avian retroviral-based vectors (e.g., particles) of the present invention can be administered as needed orally, intravenously, intra-arterially, intraperitoneally, inhalationally, intranasally, rectally, vaginally, intradermally, transdermally, intramuscularly, subcutaneously, intrathecally, intratumorally, as well as directly into specific organs including but not limited to brain, lung, liver, heart, pancreas, ovary, prostate or spleen tissue. In some embodiments, the viral vectors of the invention are delivered to the interstitial space of tissues of the animal body (including but not limited to those of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue). The interstitial space of the tissues comprises the intercellular, fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels.

For delivery of the avian retroviral-based vectors of the present invention a delivery system can be utilized. Gene delivery systems used in the art are generally reviewed in “Gene Therapy and Gene Delivery Systems (Advances in Biochemical Engineering/Biotechnology),” Schaffer and Zhou, Eds., 2006, Springer. The non-avian retroviral-based vector components of a delivery system can provide for stabilization and protection of the integrity of the DNA and assist in cellular uptake. Both the non-avian retroviral-based vector as well as the avian retroviral-based vectors components of a formulation can contribute to immune system enhancement, activation or suppression. The non-avian retroviral-based vector components of a delivery system can be selected in conjunction with a particular gene encoded by the avian retroviral-based vectors of the present invention to enhance or minimize an immune response.

The non-avian retroviral-based vector components can include transfection reagents or transfection facilitating materials. Hybrid systems have been described that exploit the advantages of both viral and non-viral gene delivery systems, include adeno-Lipofection or retro-Lipofection in which adenovirus or retrovirus infection is performed in the presence of cationic lipid vectors where classical viral transfection is poor in order to improve transgene expression. (See, e.g., Mahato and Kim (Ed.), Pharmaceutical Perspectives of Nucleic Acid-Based Therapeutics (2002) CRC Press.) Transfection agents are well known to those of skill in the art and transfection reagents can include for example but are not limited to Lipofectin, Lipofectamine, Lipofectamine 2000, Optifect and SuperFect. Transfection facilitating materials are also well known to those skilled in the art and can include for example but are not limited to lipids (including cationic lipids (e.g. DOTMA, DMRIE, DOSPA, DC-Chol, GAP-DLRIE), basic lipids (e.g., steryl amine), neutral lipids (e.g., cholesterol), anionic lipids (e.g., phosphatidyl serine) and zwittepionic lipids (e.g., DOPE, DOPC)) inorganic materials (including calcium phosphate, and metals (e.g., gold or tungsten)), particles (including “powder” type delivery solutions), peptides (including cationic peptides targeting peptides for selective delivery to certain cells or intracellular organelles including the nucleus or nucleolus), amphipathic peptides (including helix forming or pore forming peptides), basic proteins (including histones), asialoproteins, viral proteins (including Sendai virus coat protein), pore-forming proteins and polymers (include dendrimers, star-polymers, “homogenous” poly-amino acids (e.g., poly-lysine, poly-arginine), “heterogeneous” poly-ammo acids (e.g., mixtures of lysine and glycine), co-polymers, polyvinylpyrrolidinone (PVP) and polyethylene glycol (PEG)). Lipids can be mixed or combined in a number of ways to produce a variety of non-covalently bonded macroscopic structures, including for example, liposomes, multilamellar vesicles, unilamellar vesicles, micelles and simple films. For example, Brigham et al., U.S. Pat. No. 5,676,954, discloses injection of genetic material, complexed with cationic lipid earners, into mice. Further examples are disclosed in Felgner, et al., U.S. Pat. No. 5,580,859; Felgner, et al., U.S. Pat. No. 5,589,466; Wolff, et al., U.S. Pat. No. 5,693,622; Felgner, et al., U.S. Pat. No. 5,703,055, and Nabel, et al., WO 94/29469, all of which provide methods for delivering compositions comprising naked DNA or DNA cationic lipid complexes to vertebrates.

The avian retroviral-based vectors of the present invention can be administered as pharmaceutical compositions where the compound is formulated with a pharmaceutically acceptable carrier by methods well known to one skilled in the art. Techniques for formulation and administration of pharmaceutical compositions may be found, for example, in Remington: The Science and Practice of Pharmacy, 21^{st} Ed., 2005, Lippincott, Williams and Wilkins. The vectors of the present invention may be used in the manufacture of a medicament. Pharmaceutical compositions of the compounds may be formulated as solutions or lyophilized powders for parenteral administration. Such powders may be reconstituted by addition of a suitable diluent or other pharmaceutically acceptable carrier prior to use. Such powders also may be sprayed in dry form. The avian retroviral-based vectors can be prepared in liquid formulations. Such liquid formulations may be buffered, isotonic or aqueous solutions. Examples of suitable diluents contemplate for use in formulating the avian retroviral-based vectors include normal isotonic saline solution, standard 5% dextrose in water or buffered sodium or ammonium acetate solution. Such liquid formulations can be suitable for any route of administration including parenteral administration (e.g., intravenous, intramuscular, and other routes described above), oral administration or can be contained in a metered dose inhaler or nebulizer for insufflation. Such liquid compositions can also contain added excipients include for example but are not limited to polyvinylpyrrolidone, gelatin, hydroxy cellulose, acacia, polyethylene glycol, mannitol, sodium chloride or sodium citrate.

The avian retroviral-based vectors can be formulated in an appropriate salt. The term “pharmaceutically acceptable salt(s)”, as used herein, defines those salts of the avian based retroviral-based vectors which are safe and effective for topical or systemic use in vertebrates or mammals and that possess the desired biological activity. The counter ion is suitable for the intended use, non-toxic, and it does not interfere with the desired biological action of the pharmaceutical composition. Such salts may be prepared from pharmaceutically acceptable non-toxic bases including organic bases and inorganic bases. Salts derived from inorganic bases can include but are not limited to sodium, potassium, lithium, ammonium, calcium, magnesium, and the like. Salts derived from pharmaceutically acceptable organic non-toxic bases can include salts of primary, secondary, and tertiary amines, basic amino acids, and the like. Said salts include also the corresponding solvates. Pharmaceutically acceptable salts in the context of vectors of the present invention include the salts reviewed in the IUPAC Handbook of Pharmaceutically Acceptable Salts (Wermuth, C. G. and Stahl, P. H., Handbook of Pharmaceutical Salts: Properties, Selection and Use, Verlag Helvetica Chimica Acta (2002) Wiley).

The avian retroviral-based vectors (e.g., particles) can be encapsulated, tableted or prepared in a emulsion or syrup for oral administration. Pharmaceutically acceptable solid or liquid carriers can be added to enhance or stabilize the composition, or to facilitate preparation of the composition. Solid carriers can include starch, lactose, calcium sulfate dihydrate, terra alba, magnesium stearate or stearic acid, talc, pectin, acacia, agar or gelatin. Liquid carriers include syrup, peanut oil, olive oil, saline and water. For aqueous compositions used in vivo, sterile pyrogen-free water can be used. Aqueous formulations contain an effective amount of a polynucleotide together with a suitable amount of an aqueous solution in order to prepare pharmaceutically acceptable compositions suitable for administration to a vertebrate or mammal. The carrier can also include a sustained release material include glyceryl monostearate or glyceryl distearate, alone or with a wax. The amount of solid carrier varies. In some embodiments it will be between about 2 μg to about 1000 μg of retroviral particles per dosage. The pharmaceutical preparations containing the avian retroviral-based vectors are made following the conventional techniques of pharmacy involving milling, mixing, granulating, and compressing, when necessary, for tablet forms; or milling, mixing and filling for hard gelatin capsule forms. When a liquid carrier is used, the preparation may be in the form of a syrup, elixir, emulsion or an aqueous or non-aqueous suspension. For rectal administration, the compounds may be combined with excipients include cocoa butter, glycerin, gelatin or polyethylene glycols and molded into a suppository.

In some embodiments, the avian retroviral-base vectors can be administered by injectable carrier alone. The carrier can be isotonic, hypotonic or weakly hypertonic. The carrier can have a relatively low ionic strength, include for example that provided by a sucrose solution. The preparation can further comprise a source of a cytokine which is incorporated into liposomes in the form of a polypeptide or as a polynucleotide. The composition can also be administered as a bolus or slowly infused.

The avian retroviral-based vectors can be formulated to include other medically useful drugs or biological agents. The avian retroviral-based vector compositions can be administered in conjunction with the administration of other drugs or biological agents useful for the disease or condition that the compounds described herein are directed.

The compositions of the present invention containing the avian retroviral-based vectors can be delivered to a vertebrate, including a mammal, e.g., a human as described above. Administration of the compositions according to any of the methods disclosed herein can be accomplished according to any of various methods known in the art.

The avian retroviral-based vector containing compositions may be used in the manufacture of a medicament. It is understood that a pharmaceutically acceptable carrier, or a pharmaceutical composition, or any substance suitable for administration to a vertebrate or mammal should be manufactured and stored in accordance with standards of local regulations. For example many governments have guidelines or rules that regulate various aspects of the manufacture and handling of compositions which are for administration into mammals and/or humans include sanitation, process validation equipment and document traceability, and personnel qualification. In some embodiments, the compositions containing the avian retroviral-based vectors, which can include a pharmaceutical composition or a pharmaceutically acceptable carrier, are suitable for administration to a human and comply with local regulations, guidelines and/or GMPs (Good Manufacturing Practices) regulations including those set forth by the United States Food and Drug Administration for such a purpose.

The dosage of the avian retroviral-based vectors of the present invention to be administered depends to a large extent on the condition and size of the subject being treated as well as the frequency of treatment and the route of administration. Regimens for continuing therapy, including dose and frequency may be guided by the initial response and clinical judgment of the practicing physician.

As described herein, the phrase “an effective amount” refers to a dose sufficient to provide concentrations high enough to impart a therapeutically beneficial effect on the recipient thereof. The specific therapeutically effective dose level for any particular subject will depend upon a variety of factors including the disorder being treated, the severity of the disorder, the activity of the specific compound, the route of administration, the rate of clearance of the compound, the duration of treatment, the drugs used in combination or coincident with the vector and the expressed polypeptide, the age, body weight, sex, diet and general health of the subject, and like factors well known in the medical arts and sciences. Various general considerations taken into account in determining the “therapeutically effective amount” are well known to those of skill in the art and are described. (See, e.g., Goodman & Gilman's The Pharmacological Basis of Therapeutics, 11th edition, 2006, McGraw-Hill Professional.)

Determination of an effective amount is well within the capability of those skilled in the art. Generally, an efficacious or effective amount of the avian retroviral-based vectors is determined by first administering a low dose or small amount of the vector, and then incrementally increasing the administered dose or dosages until a desired effect of the therapeutic polypeptide is observed in the treated subject, with minimal or no toxic side effects. Dose can be quantified by mass amount of retroviral vector administered to a subject.

In one particularly useful embodiment, therapeutic doses are measured in IUs (1 IU equals one retroviral particle capable of integrating into a recipient genome). Exemplary therapeutically relevant dosages for administration of avian retrovirus-based vectors of the invention measured in IUs are in the range of about 1×10⁴ to about 1×10¹⁴ IU per administration, for example, about 1×10⁵ to about 1×10¹³ IU per administration, or 1×10⁶ to about 1×10¹² IU per administration, or about 5×10⁶ to about 1×10¹² IU per administration, or about 1×10⁶ to about 1×10¹¹ IU per administration, or about 1×10⁶ to about 1×10¹⁰ IU per administration, or about 1×10⁶ to about 1×10⁹ IU per administration, or about 1×10⁶ to about 1×10⁸ IU per administration, or about 5×10⁶ to about 1×10⁹ IU per administration, or about 5×10⁶ to about 5×10⁹ IU per administration. Typically, at least about 1×10⁶ IU are administered, for example, at least about 1×10⁵ IU or at least about 1×10⁶ IU, for example, 5×10⁶ IU, or 1×10⁷ IU, or 5×10⁷ IU, or 1×10⁸ IU, or 5×10⁸ IU, or 1×10⁹ IU, or 5×10⁹ IU, or 1×10¹⁰ IU, or 5×10¹⁰ IU, or 1×10¹¹ IU, or 5×10¹¹ IU, or 1×10¹² IU are administered, or a greater or lesser dosage, as needed.

The virus can be administered once or multiple times, depending on the condition being treated and the therapeutic polypeptide being administered. When administering multiple dosages, the vectors may be administered weekly, bi-weekly (i.e., about every 2 weeks), monthly, bimonthly (i.e., about every 8 weeks), or more or less often, or on an as needed basis. The vector can be administered once, twice, three, four, five, six or more times, as needed.

In one embodiment, the retroviral particles are introduced into a patient's cells (e.g., skin cells, blood cells, liver cells) after the cells are removed from the patient using standard methodologies known in the field. After removal from the patient, the cells are transduced with the retroviral particles and the cells are then reintroduced into the patient.

In one embodiment, the cells are reintroduced into the patient immediately after transduction.

In another embodiment, the cells are reintroduced into the patient after culturing of the cells, for example, to increase the number of cells. The cells can be cultured before transduction by the retrovirus or the cells can be cultured after transduction by the retrovirus. Culturing of the cells can be done for any useful amount of time. For example the cells can be culture for about 24 hours to about 7 days (e.g., about 24 hours, about 48 hours, or about 72 hours).

In one embodiment, the patient's cells are serially transduced. That is, the cells are transduced followed by an incubation period (incubation may be done simultaneously with culturing) after which the cells are transduced a second time. This transduction process can be repeated for a third time or more times, as determined to be useful by a practitioner of skill in the art. The incubation period or periods can be for any useful length of time. For example, the incubation periods(s) can be about 5 minutes to about 72 hours. In one embodiment, the incubation period is about 30 minutes to about 24 hours. In another embodiment, the incubation period is about 30 minutes to about 24 hours. In another embodiment, the incubation period is about 1 hour to about 24 hours. In another embodiment, the incubation period is about 4 hour to about 12 hours.

In one embodiment, producer cells are introduced into the patient. That is, cells producing or capable of producing therapeutic retroviral particles of the invention are introduced into the patient. For example, cells transiently transfected with DNA that provides for the making of replication deficient ALV SIN vectors lacking nucleotide sequences responsible for the gag, pol and env production are introduced into the patient. For such purpose, the invention contemplates the employment of any useful cell type. In one embodiment, the producer cells are the patient's own cells (e.g., skin cells, blood cells, liver cells) which have been removed from the patient, transiently transfected and reintroduced into the patient. In another embodiment, exogenous producer cells such as producer cells made from avian fibroblast cells (e.g., DF-1 cells) are introduced into the patient. After introduction into the patient, the producer cells will make and shed viral particles of the invention which will diffuse or circulate in the body and transduce cells of the patient's tissues.

The invention also contemplates the introduction of the transfection complexes directly into the patient's system. That is, a mixture of DNA that provides for the making of the viral particles of the invention is introduced into the patient's system along with (e.g., mixed with) transfection agents such as those described elsewhere herein (e.g., lipofectamine, DMRIE-C). Accordingly, cells within the patient's body (e.g., liver cells) will become transiently transfected with the DNA and will produce and shed viral particles of the invention which will diffuse or circulate in the body and transduce cells of the patient's tissues.

The avian retroviral-based vectors of the present invention provide replication deficient and self-inactivating vectors that can be combined with targeting methods to improve the efficacy and safety of gene therapy. The avian retroviral based vectors find use in any of the exemplary gene therapy targeting methods described herein and known in the art.

Targeting of vectors to specific cell types or tissues can be accomplished by a variety of methods well known to those of skill in the art. The avian retroviral-based vectors can be prepared as targeted forms. For example, naked viral DNA can be complexed with a nucleic acid binding domain which itself is conjugated to a cell receptor binding-internalizing ligand (see, e.g., Sosnowski, et al., WO 96/36362). The vectors can be “re-targeted” by complexing the virus with an anti-surface protein antibody or fragment thereof conjugated or genetically fused to an internalizing ligand (see, Curiel, et al., U.S. Pat. No. 5,871,727 and Sosnowski, et al., WO 98/40508). Retroviral vectors can also be modified to alter their natural tropism and target cells (see, e.g., Berkhout, et al. WO 98/51808 and Curiel, et al., U.S. Pat. No. 5,871,727) or via expression or attachment of an internalizing ligand on their surface (see, e.g., Larocca, et al., WO 99/10014).

Further strategies for targeting viral vectors to cell type specific receptors include methods wherein ligands are linked to the vector capsid through chemical (bispecific conjugates) or recombinant (genetically modified capsids) methods. Random phage display peptide libraries can be used to identify ligands binding to certain cell types in vitro or homing to tissue-specific endothelial receptors after intravenous injection in vivo. Such ligands have been used for therapeutic targeting in experimental models. Viral vectors can be redirected by means of bispecific molecular conjugates that contain targeting peptides; however, these methods can include the lack of stability of the adaptor-vector complex in vivo and immunogenicity of the adaptor molecule itself. Viral vectors can be retargeted for gene therapy methods by incorporating ligands directly into the viral capsid. Viral vectors can be retargeted by incorporation of peptides, selected by phage display methods, directly into the viral capsid. (See, e.g., Kleinschmidt, et al., US 2007/0172460.) Other examples of targeting viral vectors employ methods restricting transgene expression to the desired tissue or tumor using designer promoters for tumor targeting. (See, e.g., Nettelbeck et al., Trends in Genetics (2000) 16:174-181.)

Many examples of targeted delivery during gene therapy methods have been described. (See, e.g., Dachs, et al., Oncology Research (1997) 9:313-325.) These include for example replacing of retroviral envelope genes with heregulin encoding sequences to target virus particles to lung carcinoma cells overexpressing EGF-R and the use of polylysine and polylysine conjugates.

Many examples of targeted expression during gene therapy methods have been described. (See Dachs, et al., Oncology Research, 1997, 9:313-325.) For example, the alpha-fetoprotein (AFP) promoter/enhance can be used to specifically express a gene encoding a therapeutic polypeptide in cells expressing AFP, e.g., human hepatocellular carcinoma cells. The upstream region of the prostate specific antigen (PSA) drives expression of therapeutic polypeptide genes in prostate cells. The von Willebrand factor (vWF) promoter can be used to direct expression of therapeutic genes to the tumor vasculature, e.g., as anti-angiogenic gene therapy. The promoter for MUC1 can be used to direct expression of a gene encoding a therapeutic polypeptide to cancers that overexpress this antigen, e.g., breast cancers. The albumin enhancer element and/or promoter can be used to target therapeutic gene expression to dividing hepatocytes. The tyrosinase promoter can be used to direct expression of a therapeutic polypeptide in lung tissue, e.g., lung metastasis. The myelin basic protein promoter can be used to direct expression to glioma cells. The promoter for human carcinoembryonic antigen (CEA) can be used to direct expression of a gene encoding a therapeutic polypeptide in cancers that overexpress CEA, e.g., pancreatic carcinomas and colorectal carcinoma cells. The HER-2/neu promoter can be used to target expression of a gene encoding a therapeutic polypeptide to cancers that over express this antigen, e.g., breast and pancreatic cancers. Four repeats of the Myc-Max response element, can been used to target expression of a gene encoding a therapeutic polypeptide cancer cells that over express myc, e.g., small lung cancer and colon carcinoma. Numerous other promoters have been identified and can be used with the avian retroviral vectors of the present invention. The avian retroviral-based vectors of the present invention can contain any of the promoters listed above, as well as any promoters known to those of skill in the art.

Many examples of condition targeted expression during gene therapy methods have been described. (See, e.g., Dachs, et al., Oncology Research (1997) 9:313-325.) For example, response elements or promoters inducible by conditions including hypoxia, glucose deprivation, acidity, and ionizing radiation can be used to target expression of the genes encoding the therapeutic polypeptides. The avian retroviral-based vectors of the present invention can be modified by methods well known to those of skill in the art in order to obtain condition targeted expression during gene therapy.

The following specific examples are intended to illustrate the invention and should not be construed as limiting the scope of the claims.

### Example 1

### Vector Construction

Construction of pCMV-gagpol

pRC/CMV (Invitrogen, Inc.) was digested with Not I and Hind III and the linearized 5376 bp vector was gel purified. The gag region of the Rous Sarcoma Virus (RSV) was amplified from RSV using Pfu polymerase and the following primers: RSV-gag-1-2, GGCAAGCTTGGATCAAGCATGGAAGCCGTCATAAAGGT (SEQ ID NO:1) and RSV-gag-2, TGGGAATTCCTCCTCCTATGC (SEQ ID NO:2). The RSV PCR product was digested with EcoRI and Hind III and the 1954 bp fragment containing the gag region was gel purified. The pol region of the Rous Sarcoma Virus (RSV) was amplified with Elongase enzyme mix (Invitrogen, Inc.) using the following primers: RSV-pol1, ACACTGGGAGTCACCCGGTCAAACAG (SEQ ID NO:3) and RSV-pol2, GGGTCGACGCGGCCGCTTAACTCTCGTTGGCAGCAAG (SEQ ID NO:4). The PCR product was digested with EcoRI and NotI and a 2873 bp fragment containing the pol region was gel purified.

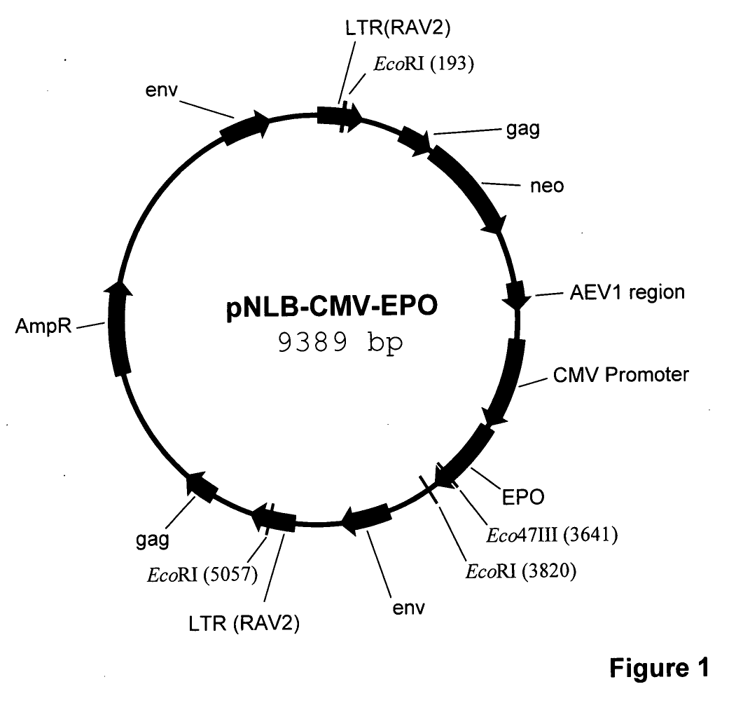

The linearized pRC/CMV, the RSV gag PCR product and the RSV pol PCR product were ligated together to produce the 10,203 bp pCMV-gagpol vector (FIG. 1).

### Construction of PNLB-CMV-EPO

pNLB-CMV-hIFN alpha-2b (see U.S. Pat. No. 6,730,822, issued May 4, 2004 and U.S. patent application Ser. No. 11/167,052, filed Jun. 24, 2005, the disclosures of which are incorporated in their entirety herein by reference) was digested with Hind III and EcoRI in order to replace the hIFN coding sequence of interest plus signal peptide coding sequence with an EPO coding sequence plus signal peptide (SEQ ID NO:11). Because multiple EcoRI and Hind III sites exist in the vector, RecA-assisted restriction endonuclease (RARE) cleavage method was used to cut the desired sites. The following oligonucleotides were used in the RARE procedure:

pnlbEcoRI3805rare

(SEQ ID NO:5)

(5′-GAC TCC TGG AGC CCG TCA GTA TCG GCG GAA TTC
CAG CTG AGC GCC GGT CGC TAC CAT TAC-3′)
and
pnlbHinD III3172rare

(SEQ ID NO:6).

(5′-TAA TAC GAC TCA CTA TAG GGA GAC CGG AAG CTT
TCA CCA TGG CTT TGA CCT TTG CCT TAC-3′)

A linearized vector of 8740 bp was obtained and was gel purified. The EPO insert was prepared by overlap PCR as follows.

The first PCR product was produced by amplification of a synthetic EPO sequence (EPO 1) cloned into a standard cloning vector with Pfu polymerase and the following primers: 5′pNLB/Epo (5′-GGGGGGAAGCTTTCACCATGGGCGTGCACGAG-3′) (SEQ ID NO:7) and pNLB/3′Epo (5′-TCCCCATACTAGACTTTTTACCTATCGCCGGTC-3′) (SEQ ID NO:8). The 2nd PCR product was produced by amplification of a region of pNLB-CMV-hIFN alpha-2b with Pfu polymerase and the following primers: 3′Epo/pNLB (5′-ACCGGCGATAGGTAAAAAGTCTAGTATGGG-3′) (SEQ ID NO:9) and pNLB/SapI (5′-GGGGGGGCTCTTCTCAGCTGGAATTCCGCCGATAC-3′) (SEQ ID NO:10). The two PCR products were mixed and reamplified with the following primers: 5′pNLB/Epo (5′-GGGGGGAAGCTTTCACCATGGGCGTGCACGAG-3′) (SEQ ID NO:7) and pNLB/SapI (5′-GGGGGGGCTCTTCTCAGCTGGAATTCCGCCGATAC-3′) (SEQ ID NO:10).

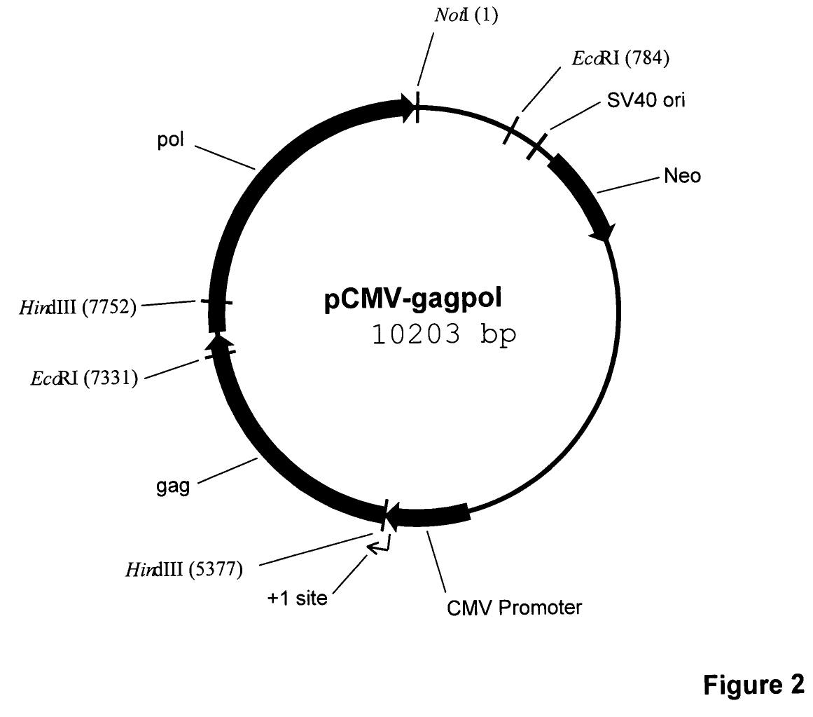

The fusion PCR product was digested with Hind III and Eco RI and a 633 bp fragment gel purified. The 8740 bp and 633 bp fragments were ligated to create pNLB-CMV-EPO (FIG. 2).

EPO 1-Synthetic EPO sequence (610 nt)

(SEQ ID NO:11)

AAGCTTTCACCATGGGCGTGCACGAGTGCCCTGCTTGGCTGTGGCTGCTC
TTGAGCCTGCTCAGCCTGCCTCTGGGCCTGCCTGTGCTGGGCGCTCCTCC
AAGGCTGATCTGCGATAGCAGGGTGCTGGAGAGGTACCTGCTGGAGGCTA
AGGAGGCTGAGAACATCACCACCGGCTGCGCTGAGCACTGCAGCCTGAAC
GAGAACATCACCGTGCCTGATACCAAGGTGAACTTTTACGCTTGGAAGAG
GATGGAGGTGGGCCAGCAGGCTGTGGAGGTGTGGCAGGGCCTGGCTCTGC
TGAGCGAGGCTGTGCTGAGGGGCCAGGCTCTGCTGGTGAACAGCTCTCAG
CCTTGGGAGCCTCTGCAGCTGCACGTGGATAAGGCTGTGAGCGGCCTGAG
AAGCCTGACCACCCTGCTGAGGGCTCTGAGGGCTCAGAAGGAGGCTATCA
GCCCTCCAGATGCTGCAAGCGCTGCCCCTCTGAGGACCATCACCGCTGAT
ACCTTTAGGAAGCTGTTTAGGGTGTACAGCAACTTTCTGAGGGGCAAGCT
GAAGCTGTACACCGGCGAGGCTTGCAGGACCGGCGATAGGTAAAAAGGCC
GGCCGAGCTC

Construction of pNLB-CMV-Des-Arg166-EPO

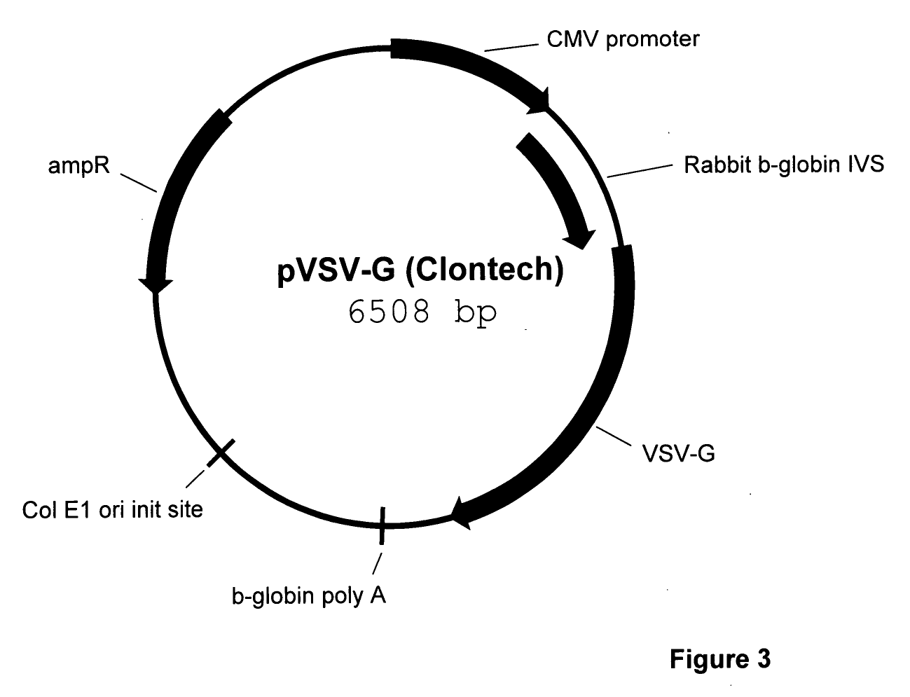

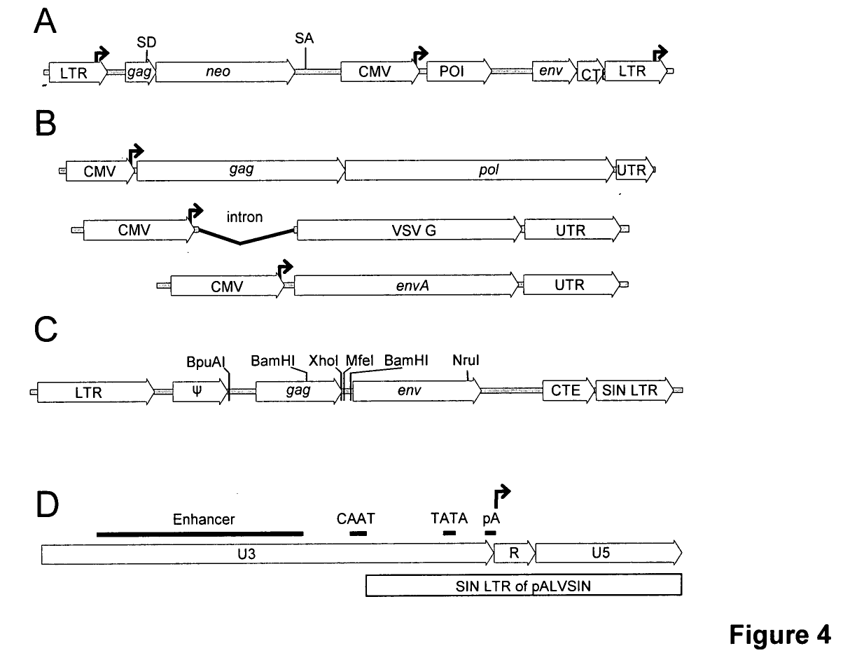

An EPO coding sequence is produced which codes for a 165 amino acid form of EPO with the terminal codon (coding for arginine at position 166) removed. A 179 bp region of pNLB-CMV-EPO corresponding to the sequence that extends from an Eco 47III site that resides in the EPO coding sequence to an EcoRI site that resides downstream of the EPO stop codon in pNLB-CMV-EPO was synthesized with the terminal arginine codon (position 166) eliminated so that aspartic acid (amino acid 165) will be the terminal amino acid codon, resulting in a 176 bp Eco 47111/EcoRI fragment. The fragment was synthesized by Integrated DNA Technologies (Coralville, Iowa 52241) and cloned into a pDRIVE vector (Qiagen, Inc), creating pDRIVE-des-Arg166-EPO (FIG. 3). The 176 bp Eco 47III/EcoRI fragment was subcloned into the Eco47III/EcoRI site of pNLB-CMV-EPO, creating pNLB-CMV-Des-Arg166-EPO (FIG. 4).

### Example 2

### Transient Transfection of DF-1 Cells

The day before transfection, 3.7×10⁶ DF-1 cells were plated in 150 mm tissue culture dishes in DF-1 media (Dulbecco's Modified Eagle Medium with high glucose, L-glutamine, pyridoxine HCl, 10% fetal bovine serum, 10 U/ml penicillin G and 10 ug/ml streptomycin) and cultured at 37° C. with 6% CO2. The next day the cells were transfected as follows. Each plate was washed with 6 ml OptiMEM (Invitrogen, Inc.) and refed with 5 ml OptiMEM. 18.4 ug of the retrovector, pNLB-CMV-Des-Arg166-EPO, 18.4 ug of pCMV-gagpol and 0.92 ug of pVSV-G were mixed in 4.6 ml OptiMEM in a 15 ml polystyrene tube or bottle. 110 ul of DMRIE-C was mixed with 4.6 ml OptiMEM. The lipid/OptiMEM was added to the DNA/optiMEM. After mixing by inverting or swirling, the transfection mix was incubated at RT for 15 minutes and then added to one 150 mm plate. The plate was incubated at 37° C. with 6% CO2 for 3 to 4 hours. The transfection mix was removed, the plate was washed once with 6 ml DF-1 media and refed with 20 ml DF-1 media. In certain instances sodium butyrate may be added at this stage (for example, about 2 mM to about 40 mM) and the cells incubated overnight. In such case, the medium is removed the next morning and the cells are again washed with DF-1 media. Such treatment with sodium butyrate can increase the viral particle titer about 5 to 10 fold over the titer that would otherwise be obtained without use of sodium butyrate. The plate was incubated at 37° C. with 6% CO2 for 18 to 60 hours and the media from the plate harvested by pouring into and filtering through a Millipore SteriCup Vacuum Filter, 0.45 um PVDF 250 ml (cat no. SCHV U02 RE).

Filtered viral media from two transfected 150 mm plates was poured into Beckman SW28 Ultraclear tubes (cat no. 344058). The media was centrifuged in a SW28 rotor at 19.4 krpm, for 2 hours at 4° C. Most of the super was removed and DF-1 media filtered with a 0.2 uM filter was added to a final volume of 100 to 400 ul. The viral pellet was resuspended at 4° C. for 1 to 4 hrs or overnight. The media and pellet were further resuspended by triturating with a Gilman P200 pipettor 3-4 times and the viral resuspension was transferred to a Nunc Cryo vial and frozen at −70° C. To titer, aliquots of the viral resuspension were thawed in 37° C. water bath, diluted with DF-1 media and plated on Senta or DF-1 cells. One to two days later, media containing G418 at 200 ug/ml was added to the Senta or DF-1 cells. Media was changed every two to three days and colonies were counted when evident. Titer of concentrated virus was approximately 1×10⁷ (without sodium butyrate treatment) which is approximately a 10 fold higher titer than typically obtained using traditional methods to produce replication deficient retroviral particles, such as the methods disclosed in U.S. Pat. No. 6,730,822, issued May 4, 2004, the disclosure of which is incorporated in its entirety by reference, which discloses the use of Senta and Isolde cells for the production of NLB replication deficient retroviral vectors.

### Example 3

### Production of Transgenic Birds

7 ul of the virus suspension prepared according to Example 2 was injected into the subgerminal cavity of 97 fertile, unincubated White Leghorn eggs (Charles River, SPAFAS). 54 chicks hatched and were reared to sexual maturity. Semen was collected and DNA extracted by the Chelex method. 100 ng of sperm DNA, as determined by the PicoGreen assay (Molecular Probes) was assayed for the presence of the EPO transgene using the Applied Biosystems TaqMan® Fast Universal PCR Master Mix and the Applied Biosystems 7900HT. The primers were: SJ-EPO-for, 5′-GCCCTCCAGATGCTGCAA-3′ (SEQ ID NO:12) and SJ-EPO-rev, 5′-CCCTAAACAGCTTCCTAAAGGTATCA-3′ (SEQ ID NO:13). The Taqman EPO probe sequence was 5′-CGCTGCCCCTCTGAGGACCATC-3′ (SEQ ID NO:14) and was labeled with FAM (6-carboxyfluorescin) at the 5′ end and TAMRA (N,N,N′,N′-tetramethyl-6-carboxyrhodamine) at the 3′end. One rooster was found to have a significant level of the EPO gene in his semen. This rooster was bred to wildtype hens. Approximately 144 chicks were hatched. Their blood DNA was extracted and tested for the presence of the transgene using the EPO Taqman assay. Two chicks were found to be positive for the transgene. The quantity of the transgene was such that every cell would be calculated to have one copy of the EPO transgene, as would be expected for a G1.

### Example 4

### Treatment of Metastatic Melanoma by Delivery of IFN-γ by a Replication-Deficient ASV Vector

A patient with metastatic melanoma is administered interferon-γ (IFN-γ) via intratumoral injections of 1×10⁷ plaque forming units/mL/day (for five days at 0.5 mL per injection) of a replication-deficient ASV vector designed to express IFN-γ under the control of an internal simian virus 40 promoter. The ASV vector particles are produced essentially according to the method of Example 2. The observation of the improvement of local regional responses as measured by decreased or stabilized tumor sizes, as well as increased life expectancy, after administration of the ASV vector compared to the symptoms before administration of the ASV vector indicates the successful treatment of the patient for metastatic melanoma. (See, e.g., Fujii, et al., Cancer Gene Therapy (2000) 7(9):1220-1230.)

### Example 5

### Treatment of Malignant Pleural Mesothelioma by Delivery of Interferon Beta (IFN-β) by a Replication-Deficient and Self-Inactivating ASLV Vector

A patient with malignant pleural mesothelioma disease is administered interferon beta (IFN-β) via instillation through a tunneled intrapleural catheter as a single dose of 50 cc containing 9×10¹¹ particles of a replication-deficient and self-inactivating ASLV vector designed to express IFN-β under the control of a CMV promoter. The ASLV vector particles are produced essentially according to the method of Example 2. The observation of meaningful clinical responses of disease stability and regression, as measured by 18FDG-PET and CT scans, at day 60 after administration of the ASLV vector compared to the symptoms before administration of the ASLV vector indicates the successful treatment of the patient for malignant pleural mesothelioma. (See, e.g., Sterman, et al., Clinical Cancer Research (2007) 13(15):4456-4466.)

### Example 6

### Treatment of Fabry Disease by Delivery of Alpha-Galactosidase A by a Replication-Deficient and Self-Inactivating ALV Vector

A patient with Fabry disease is administered alpha-galactosidase A intranasally in a 100 μL solution (PBS/5% sucrose solution of virus/DEAE-dextran (500 kDa)) containing 10¹⁰ particles of a replication-deficient and self-inactivating ALV vector designed to express alpha-galactosidase A under the control of a beta-actin promoter wherein the vector particles are produced essentially according to the method of Example 2. The observation of increased alpha-galactosidase A expression in the lungs in conjunction with increased globotriaosylceramide (GL-3) clearing in the patient, after administration of the ALV vector compared to the symptoms before administration of the ALV vector indicates the successful treatment of the patient for Fabry disease. (See, e.g., Li, et al., Molecular Therapy (2002) 5(6):745-754 and gene therapy human clinical trial information located on the World Wide Web at wiley.co.uk/genmed/clinical/.)

### Example 7

### Treatment of Hemophilia/Haemophilia A by Delivery of Factor VIII by a Replication-Deficient and Self-Inactivating ALV Vector

A patient with Haemophilia A is administered human Factor VIII (B-domain-deleted gene) (FVIII) via peripheral vein infusion a solution containing 9.2×10⁷ transducing units/kg (TU/kg) (administered by 3 equal daily doses over 3 consecutive days) of a replication-deficient and self-inactivating ALV vector, produced essentially according to the method of Example 2, designed to express FVIII under the control of a Moloney murine leukemia virus (MoMLV) 5′ LTR promoter. The observation of decreased bleeding frequency as recorded by the patient, as well as increased active FVIII levels in the blood (measured by coagulation assays), after administration of the ALV vector compared to before administration of the ALV vector indicates the successful treatment of the patient for Haemophilia A. (See, e.g., Powell, et al., Blood (2003) 102(6):2038-2045.)

### Example 8

### Treatment of Severe Combined Immunodeficiency Disease (SCID)-X1 Disease by Delivery of γc by a Replication-Deficient and Self-Inactivating ALV Vector

A patient with Severe Combined Immunodeficiency Disease (SCID)-X1 is administered by infusion 17×10⁶ cells/kg CD34⁺ cells infected with the ALV vector designed to express γc under the MFG vector promoter (CD34⁺ cells are infected with ALV over 3 days prior to administration). ALV vector particles are produced essentially according to the method of Example 2. Clinical improvement, demonstrated by the disappearance of protracted diarrhea and skin lesions, as well as the patient leaving isolation and remaining at home with no treatment for approximately 1 year after administration of the ALV vector as compared to the presence of lesions and diarrhea, as well as mandatory isolation before administration of the ALV vector, indicates the successful treatment of the patient for SCID-X1. (See, e.g., Cavazzana-Calvo, et al., Science (2000) 288(5466):669-672.)

### Example 9

### Treatment of HIV Infection by Delivery of CD4 Zeta Chimeric Receptor Expressing Cells by Administration of T-Cells Infected with a Replication-Deficient and Self-Inactivating FSV Vector Coding for the Cd4 Zeta Chimeric Receptor

A patient that is HIV-seropositive is administered a chimeric CD4ζ receptor comprising a human CD4 extracellular and transmembrane domain linked to the cytoplasmic domain for the CD3 T-cell receptor zeta (ζ) chain via a single intravenous in fusion of 3×10¹⁰ CD4ζ receptor modified T cells containing a replication-deficient and self-inactivating FSV vector designed to express the CD4ζ chimeric receptor under the control of a murine maloney leukemia virus (MMLV) promoter. FSV vector particles are produced essentially according to the method of Example 2. The observation of increased antiviral activity of the CD4ζ chimeric receptor modified T cells, as evidenced by a decrease in plasma viral load after administration of the FSV vector compared to the viral load before administration of the FSV vector, indicates the successful treatment of the patient for HIV infection. (See, e.g., Mitsuyasu, et al., Blood (2000) 96(3):785-793.)

### Example 10

### Treatment of Mycobacterium Tuberculosis by Delivery of Ag85 by a Replication-Deficient ALV Vector

A patient with Mycobacterium Tuberculosis is administered Ag85A, in conjunction with INH therapy, via intramuscular injection of about 1×10⁶ to about 1×10⁸ IUs (3 times at 4 week intervals) of a replication-deficient ALV vector designed to express Ag85A under the control of a functional ALV LTR promoter. ALV vector particles are produced essentially according to the method of Example 2. The observation that no viable bacteria are recovered from the patient immunized with Ag85A demonstrates the ALV vector provides a useful adjuvant to INH therapy and indicates the successful treatment of the patient for M. tuberculosis reactivation. (See, e.g., Ha, et al., Gene Therapy (2003) 10:1592-1599 and gene therapy clinical trial information located on the World Wide Web at wiley.co.uk/genmed/clinical/.)

### Example 11

### Treatment of Peripheral Artery Disease by Delivery of VEGF by a Replication-Deficient and Self-Inactivating ALV Vector

A patient with peripheral artery disease is administered vascular endothelial growth factor (VEGF) via intramuscular injection of 4 mg (given in two doses 4 weeks apart) of a replication-deficient and self-inactivating ALV vector designed to express VEGF under the control of an immediate early CMV promoter. ALV vector particles are produced essentially according to the method of Example 2. The observation of the regression of resting pain and improved tissue integrity in the ischemic limb, as well as decreased limb pain, decreased requirement for limb amputation and increased angiogenesis, after administration of the ALV vector compared to the symptoms before administration of the ALV vector indicates the successful treatment of the patient for peripheral arterial disease. (See, e.g., Kim, et al., Experimental and Molecular Medicine (2004) 36(4):336-344.)

### Example 12

### Treatment of Coronary Artery Disease by Delivery of VEGF by a Replication-Deficient and Self-Inactivating ALV Vector

A patient with coronary artery disease is administered vascular endothelial growth factor (VEGF) via direct intramyocardial injection of 250 μg (2 mL over four sites) of a replication-deficient and self-inactivating ALV vector designed to express VEGF under the control of a CMV promoter. ALV vector particles are produced essentially according to the method of Example 2. The observation of the reduction in angina frequency and reduced nitroglycerin consumption after ALV administration indicates that when compared to the symptoms before administration of the ALV vector indicates successful treatment of the patient for coronary artery disease. (See, e.g., Lathi, et al., Anesthesia & Analgesia (2001) 92:19-25.)

All documents (e.g., U.S. patents, U.S. patent applications, publications) cited in the above specification are herein incorporated by reference. Various modifications and variations of the present invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A method comprising:
transiently introducing into a cell nucleotide sequence encoding an avian retroviral vector containing a nucleotide sequence encoding a therapeutic protein wherein the avian retroviral vector is replication deficient;
transiently introducing into the cell nucleotide sequence encoding products required for replication of the replication deficient retroviral vector, the products being at least two of gag, pol and env proteins;
harvesting a viral particle; and
introducing the retroviral vector of the viral particle into a cell of a vertebrate.

2. The method of claim 1 wherein the vertebrate is an avian.

3. The method of claim 1 wherein the vertebrate is a chicken.

4. The method of claim 1 wherein the products required for replication of the replication deficient retroviral vector are gag, pol and env proteins.

5. The method of claim 1 wherein the avian retroviral vector is a SIN vector.

5. The method of claim 1 wherein each introducing is facilitated by transfection.

6. The method of claim 1 wherein the cell is a fibroblast cell.

7. The method of claim 1 wherein the cell is an avian cell.

8. The method of claim 1 wherein the cell is a chicken cell.

9. The method of claim 1 wherein the cell is a DF-1 cell.

10. The method of claim 1 wherein the nucleotide sequence encoding a retroviral vector encodes a retroviral vector based on a retrovirus selected from the group consisting of Avian Leukemia/Leukosis Viruses (ALV), RAV-0, RAV-1, RAV-2, Avian Sarcoma Viruses (ASV), Avian Sarcoma/Acute Leukemia Viruses (ASLV), Rous Sarcoma Virus (RSV), Fujinami Sarcoma Viruses (FSV), Avian Myeloblastosis Viruses (AMV), Avian Erythroblastosis Viruses (AEV), Avian Myelocytomatosis Viruses (MCV), MC29, Reticuloendotheliosis Viruses (REV) and Spleen Necrosis Virus (SNV).

11. The method of claim 1 wherein the nucleotide sequence encoding a retroviral vector encodes a retroviral vector based on Avian Leukemia/Leukosis Viruses (ALV).

12. The method of claim 1 wherein the nucleotide sequence encoding products required for replication of the replication deficient retroviral vector is nucleotide sequence from a retrovirus selected from the group consisting of Avian Leukemia/Leukosis Viruses (ALV), RAV-0, RAV-1, RAV-2, Avian Sarcoma Viruses (ASV), Avian Sarcoma/Acute Leukemia Viruses (ASLV), Rous Sarcoma Virus (RSV), Fujinami Sarcoma Viruses (FSV), Avian Myeloblastosis Viruses (AMV), Avian Erythroblastosis Viruses (AEV), Avian Myelocytomatosis Viruses (MCV), MC29, Reticuloendotheliosis Viruses (REV) and Spleen Necrosis Virus (SNV), or combinations thereof.

13. The method of claim 1 wherein the retrovirus contains a coding sequence for an exogenous protein operably linked to a promoter.

14. The method of claim 1 wherein the therapeutic protein is selected form the group consisting of immunoglobulin, enzyme, fusion protein and cytokine.

15. The method of claim 1 wherein the therapeutic protein is a human protein.

16. The method of claim 1 wherein the vertebrate cells are embryonic cells.

17. A method comprising:
transiently introducing into an avian cell a nucleotide sequence encoding an avian retroviral vector wherein the avian retroviral vector is replication deficient;
transiently introducing into the avian cell one or more nucleotide sequences wherein the nucleotide sequence(s) encode products required for replication of the replication deficient retroviral vector the products being gag, pol and env proteins; and
harvesting viral particles.

18. The method of claim 17 comprising transducing the harvested viral particles into a vertebrate cell.

19. The method of claim 17 wherein the vertebrate cell is an avian embryonic cell.

20. The method of claim 17 wherein the avian cell is a chicken cell.

21. The method of claim 17 wherein the avian cell line is a chicken fibroblast cell.

22. The method of claim 17 wherein the avian cell line is a DF-1 cell.

23. The method of claim 17 wherein the nucleotide sequence encoding a retroviral vector is based on a retrovirus selected from the group consisting of Avian Leukemia/Leukosis Viruses (ALV), RAV-0, RAV-1, RAV-2, Avian Sarcoma Viruses (ASV), Avian Sarcoma/Acute Leukemia Viruses (ASLV), Rous Sarcoma Virus (RSV), Fujinami Sarcoma Viruses (FSV), Avian Myeloblastosis Viruses (AMV), Avian Erythroblastosis Viruses (AEV), Avian Myelocytomatosis Viruses (MCV), MC29, Reticuloendotheliosis Viruses (REV) and Spleen Necrosis Virus (SNV).

24. A method comprising:
introducing into a cell a nucleotide sequence encoding an avian retroviral vector wherein the avian retroviral vector is replication deficient and contains a coding sequence for an exogenous protein;
introducing into the cell a nucleotide sequence under the control of a promoter that is functional in the cell wherein the nucleotide sequence encodes products required for replication of the replication deficient retroviral vector, the products being at least two of the gag, pol and env proteins;
harvesting viral particles;
introducing the harvested particles into an avian blastodermal cell;
obtaining a transgenic avian which has developed from the blastodermal cell wherein the avian produces the exogenous protein which is present in an egg laid by the transgenic avian; and
isolating the protein from the egg.

25. The method of claim 24 wherein the products are gag, pol and env.

26. The method of claim 24 wherein the retroviral vector is an ALV based vector.

27. The method of claim 24 wherein the vector is a SIN vector.

28. The method of claim 24 wherein the env protein is a VSV envelope protein.

29. The method of claim 24 wherein the avian is a chicken.

30. A method comprising:
administering to a vertebrate cell in vivo a replication deficient retroviral vector comprising a nucleotide sequence encoding a therapeutic polypeptide, wherein the avian retroviral vector is produced in DF-1 cells and integrates into the genome of a vertebrate target cell and expresses the nucleotide sequence encoding a therapeutic protein.

31. The method of claim 30 wherein the vertebrate is an avian.

32. The method of claim 30 wherein the polynucleotide encoding the therapeutic polypeptide is operably linked to a constitutive promoter.

33. The method of claim 30 wherein the polynucleotide encoding the therapeutic polypeptide is operably linked to a tissue specific promoter.

34. The method of claim 30 wherein the polynucleotide encoding the therapeutic polypeptide is operably linked to an inducible promoter.

35. The method of claim 30 wherein the avian retroviral vector is devoid of functional gag, pol, and env coding sequences.

36. The method of claim 30 wherein the retroviral vector has a 5′ LTR and a 3′ LTR at least one of which is transcriptionally inactive upon integration into the vertebrate target cell.

37. The method of claim 30 wherein the avian retroviral vector is pseudotyped with a vesicular stomatitis virus G-protein (VSV-G).


SEQUENCE LISTING (25 sequences)

SEQ ID NO: 1 - DNA, 38 bp, Rous sarcoma virus
ggcaagcttggatcaagcatggaagccgtcataaaggt

SEQ ID NO: 2 - DNA, 21 bp, Rous sarcoma virus
tgggaattcctcctcctatgc

SEQ ID NO: 3 - DNA, 26 bp, Rous sarcoma virus
acactgggagtcacccggtcaaacag

SEQ ID NO: 4 - DNA, 37 bp, Rous sarcoma virus
gggtcgacgcggccgcttaactctcgttggcagcaag

SEQ ID NO: 5 - DNA, 60 bp, Avian leukosis virus
gactcctggagcccgtcagtatcggcggaattccagctgagcgccggtcgctaccattac

SEQ ID NO: 6 - DNA, 60 bp, Avian leukosis virus
taatacgactcactatagggagaccggaagctttcaccatggctttgacctttgccttac

SEQ ID NO: 7 - DNA, 32 bp, Homo sapiens
ggggggaagctttcaccatgggcgtgcacgag

SEQ ID NO: 8 - DNA, 33 bp, Homo sapiens
tccccatactagactttttacctatcgccggtc

SEQ ID NO: 9 - DNA, 30 bp, Homo sapiens
accggcgataggtaaaaagtctagtatggg

SEQ ID NO: 10 - DNA, 35 bp, Avian leukosis virus
gggggggctcttctcagctggaattccgccgatac

SEQ ID NO: 11 - DNA, 610 bp, Homo sapiens
aagctttcaccatgggcgtgcacgagtgccctgcttggctgtggctgctcttgagcctgctcagcctgcctctgggcctgcctgtgctgggcgctcctccaaggctgatctgcgatagcagggtgctggagaggtacctgctggaggctaaggaggctgagaacatcaccaccggctgcgctgagcactgcagcctgaacgagaacatcaccgtgcctgataccaaggtgaacttttacgcttggaagaggatggaggtgggccagcaggctgtggaggtgtggcagggcctggctctgctgagcgaggctgtgctgaggggccaggctctgctggtgaacagctctcagccttgggagcctctgcagctgcacgtggataaggctgtgagcggcctgagaagcctgaccaccctgctgagggctctgagggctcagaaggaggctatcagccctccagatgctgcaagcgctgcccctctgaggaccatcaccgctgatacctttaggaagctgtttagggtgtacagcaactttctgaggggcaagctgaagctgtacaccggcgaggcttgcaggaccggcgataggtaaaaaggccggccgagctc

SEQ ID NO: 12 - DNA, 18 bp, Homo sapiens
gccctccagatgctgcaa

SEQ ID NO: 13 - DNA, 26 bp, Homo sapiens
ccctaaacagcttcctaaaggtatca

SEQ ID NO: 14 - DNA, 22 bp, Homo sapiens
cgctgcccctctgaggaccatc

SEQ ID NO: 15 - DNA, 1458 bp, Artificial Sequence
  pALV-SIN
aatgtagtcttatgcaatactcttgtagtcttgcaacatgcttatgtaacgatgagttagcaacatgccttataaggagagaaaaagcaccgtgcatgccgattggtgggagtaaggtggtatgatcgtggtatgatcgtgccttgttaggaaggcaacagacgggtctaacacggattggacgaaccactgaattccgcattgcagagatattgtatttaagtgcctagctcgatacaataaacgccatttgaccattcaccacattggtgtgcacctgggttgatggccggaccgttgattccctgrcgactacgagcacatgcatgaagcagaaggcttcatttggtgaccccgacgtgatcgttagggaatagtggtcggccacaggcggcgtggcgatcctgtcctcatccgtctcgcttattcggggagcggacgatgaccctagtagagggggctgcggcttaggagggcagaagctgagtggcgtcggagggagccctactgcagggggccaacataccctaccgagaactcagagagtcgttggaagacgggaaggaagcccgacgactgagcggtccaccccaggcgtgattccggttgctctgcgtgattccggtcgcccggtggatcaagcatggaagccgtcataaaggtgatttcgtccgcgtgtaagacctattgcgggaaaacctctccttctaagaaggaaataggggctatgttgtccctgttacaaaaggaagggttgcttacgtccccctcagacttatattccccggggtcctgggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaggaatataaaaaaattacaggaggcttataagcagcccgaaagaagagcgtaggcgagttcttgtattccgtgtgatagctggttggattggtaattgatcggctggcacgcggaatataggaggtcgctgaatagtaaacttgtagacttggctacagcatagagtatcttctgtagctctgatgactgctaggaaataatgctacggataatgtggggagggcaaggcttgcgaatcgggttgtaacgggcaaggcttgactgaggggacaatagcatgtttaggcgaaaagcggggcttcggttgtacgcggttaggagtcccctcaggatatagtagtttcgcttttgcatagggagggggacggattggacgaaccactgaattccgcattgcagagatattgtatttaagtgcctagctcgatacaataaacgccatttgaccattcaccacattggtgtgcacctgggttgatggccggaccgttgattccctgrcgactacgagcacatgcatgaagcagaaggcttcatt

SEQ ID NO: 16 - DNA, 6508 bp, Artificial Sequence
  pVSV-G
gcggccgctctagagagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggtcgaccgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctctttcctacagctcctgggcaacgtgctggttgttgtgctgtctcatcattttggcaaagaattcctcgacggatccctcgaggaattctgacactatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaactcaaatcctgcacaacagattcttcatgtttggaccaaatcaacttgtgataccatgctcaaagaggcctcaattatatttgagtttttaatttttatgaaaaaaaaaaaaaaaaacggaattcctcgagggatccgtcgaggaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacggatcggccgcaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcgggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtg

SEQ ID NO: 17 - DNA, 10203 bp, Artificial Sequence
  pCMV-gagpol
ggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaaccagctggggctcgaggggggatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgcctttactgagcactctttaatagtggactcttgttccaaactggaacaacactcaaccctatctcggtctattcttttgatttataagatttccatcgccatgtaaaagtgttacaattagcattaaattacttctttatatgctactattcttttggcttcgttcacggggtgggtaccgagctcgaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttggatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctggatcccgtcgacctcgagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagaccggaagcttggatcaagcatggaagccgtcataaaggtgatttcgtccgcgtgtaagacctattgcgggaaaacctctccttctaagaaggaaataggggctatgttgtccctgttacaaaaggaagggttgcttatgtctccctcagacttatattccccggggtcctgggatcccattaccgcggcgctctcccagcgggcaatggtacttggaaaatcgggagagttaaaaacctggggattggttttgggggcattgaaggcggctcgagaggaacaggttacatctgagcaagcaaagttttggttgggattagggggagggagggtctctcccccaggtccggagtgcatcgagaaaccagctacggagcggcgaatcgacaaaggggaggaggtgggagaaacaactgtgcagcgagatgcaaagatggcgccggaggaaacggccacacctaaaaccgttggcacatcctgctatcattgcggaacagctattggctgtaattgcgccacagcctcggcccctcctcctccttacgtggggagtggtttgtatccttccctggcgggggtgggagagcagcagggccaggggggtgacacacctcggggggcggaacagccaagggcggagccagggcacgcgggtctggcccctgggccggccctgactgactgggcaaggatcagggaggagcttgcgagtacaggtccgcccgtggtggccatgcctgtagtgattaagacagagggacccgcttggacccctctggagccaaaattgatcacaagactggctgatacggtcaggaccaagggcttacgatccccgatcactatggcagaagttgaagcgcttatgtcttccccgctgctgccgcatgacgttacgaatctaatgagagttattttaggacctgccccatatgccttatggatggacgcttggggagtccaactacagacggttatagcggcagccactcgcgacccccgacatccagcgaacggtcaagggcggggggaacggactaacttggatcgcttaaagggcttagctgatgggatggtgggcaacccacagggtcaggccgcattattgagaccgggggaattggttgctattacggcgtcggctctccaggcgtttagagaggttgcccgattggcggaacctgcaggtccatgggcggacatcacgcagggaccatctgagtcctttgttgattttgccaatcggcttataaaggcggttgaggggtcagatctcccgccttccgcacgggctccggtgatcattgactgctttaggcagaagtcacagccagatattcagcagcttatacgggcagcaccctctacgctgaccaccccaggagagataatcaaatatgtgctagacaggcagaagattgcccctcttacggatcagggcatagctgcggccatgtcgtctgctattcagcccttagttatggcagtagtcaatagagagagggacggacaaactgggtcgggtggtcgtgcccgaaggctctgctacacttgtggatccccgggacattatcaggcgcagtgcccgaaaaaacgaaagtcaggaaacagccgtgagcgatgtcagctgtgtgacgggatggggcacaacgctaaacagtgtaggagacgggatagcaaccagggacagcgcccaggaagaggcctctcttcggggccatggcctgtctctgagcagcctgctgtttcgttagcgatgacaatggaacataaagatcgccccttggttagggtcatcctgactaacactgggagtcacccggtcaaacagcgttcggtgtatatcaccgcgctgttggactctggagcggacatcactattatttcagaggaggactggcctacggattggccggtggtggataccgcgaacccacagattcacggcataggaggaggaattcccatgcgaaagtctcgggacatgatagagttgggggttattaaccgagacgggtcgttggagcgacccctgctccttttccccgccgtagctatggttagggggagtatcctaggaagagattgtctgcagggcctagggctccgcttgacaaatttgtagggagggccactgttcttactgttgcgctacatctggctattccgctcaaatggaagccagaccacacgcctgtgtggattgaccagtggccccttcctgaaggtaaacttgtagcgctaacgcaattagtggaaaaagaattacagttaggacatatagaaccctcacttagctgttggaacacacctgtctttgtgatccggaaggcttccgggtcttatcgcttattgcatgacttacgcgctgttaacgccaagcttgttccttttggggctgtccaacagggggcgccagttctctccgcgctcccgcgtggctggcccctgatggttctagacctcaaggattgcttcttttctattcctcttgcggaacaagatcgcgaggcttttgcatttacgctcccctctgtgaataaccaggcccccgctcgaagattccaatggaaggtcttgccccaagggatgacctgttctcccactatctgtcagttggtagtgggtcaggtacttgagcccttgcgactcaagcacccatctttgcgcatgttgcattatatggatgatcttttgctagccgcctcaagtcatgatgggttggaagcggcgggggaagaggttatcaatacattggaaagagccgggttcaccatttcgcctgataagatccagagggagcccggagtacaatatcttgggtacaagttaggcagtacgtatgtagcacccgtaggcctggtagcagaacccaggatagccaccttgtgggatgttcaaaagctggtggggtcacttcagtggcttcgcccagcgttaggaatcccgccacgactgatgggccccttttatgagcagttacgagggtcagatcctaacgaggcgagggaatggaatctagacatgaaaatggcctggagagagatcgtacagctcagcaccacggctgccttggaacgatgggaccctgccctgcctctggaaggagcggtcgttagatgtgaacagggggcaataggggtcctgggacagggactgtccacacacccaaggccatgtttgtggttattctccacccaacccaccaaggcgtttactgcttggttagaagtgctcacccttttgattactaagctacgcgcttcggcagtgcgaacctttggcaaggaggttgatatcctcctgttgcctgcatgcttccgggaggaccttccgctcccggaggggatcctgttagcccttagggggtttgcaggaaaaatcaggagtagtgacacgccatctatttttgacatagcgcgtccactgcatgtttctctgaaagtgagggttaccgaccaccctgtaccgggacccactgtctttactgacgcctcctcaagcacccataagggggtggtagtctggagggagggcccaaggtgggagataaaagaaatagctgatttgggagcaagtgtacaacaactggaagcacgcgctgtggccatggcacttctgctgtggccgacaacgcccactaatgtagtgactgactccgcgtttgttgcgaaaatgttactcaagatggggcaggagggagtcccgtctacagcggcggcctttattttagaggatgcgttaagccaaaggtcagccatggccgccgttctccacgtgcggagtcattctgaagtgccagggtttttcacagaaggaaatgacgtggcagatagtcaagccacctttcaagcgtatcccttgagagaggctaaagatcttcatactactctccatattggaccccgcgcgctatccaaagcgtgtaatatatctatgcagcaggctagggaggttgttcagacctgcccgcattgtaattcagcccctgcgttggaggccggggtaaatcctaggggtttgggacccctacagatatggcagacagactttacgcttgagcctagaatggccccccgttcctggctcgctgtcactgtggataccgcctcatcggcgatagtcgtaactcagcatggccgtgtcacatcggttgctgcacaacatcattgggccacggctatcgccgttttgggaagaccaaaggccataaaaacagataacgggtcctgcttcacgtctaaatccacgcgggagtggctcgcgagatgggggatagcacacaccaccgggattccgggaaattcccagggtcaagctatggtagagcgggccaaccggctcctgaaagataagatccgtgtgcttgcggaaggggacggctttatgaaaagaatccccgccagcaaacagggggaactactagccaaagcaatgtatgccctcaatcactttgagcgtggtgaaaacacgaaaacaccggtacaaaaacactggagacctaccgttcttacagaaggacccccggttaaaatacgaatagagacaggggagtgggaaaaaggatggaacgtgctagtctggggacgaggttatgccgctgtgaaaaacagggacactgataaggttatttgggtaccctctcgaaaggttaaaccggacatcacccaaaaggatgaggtgactaagaaagatgaggcgagccctctttttgcaggcagttctgactggataccctggggagacgagcaagaaggactccaagaagaagccgccagcaacaagcaagaaggacccggagaagacacccttgctgccaacgagagttaagc

SEQ ID NO: 18 - DNA, 9384 bp, Artificial Sequence
  pNLB-CMV-EPO
aatgtagtcttatgcaatactcttgtagtcttgcaacatgcttatgtaacgatgagttagcaacatgccttataaggagagaaaaagcaccgtgcatgccgattggtgggagtaaggtggtatgatcgtggtatgatcgtgccttgttaggaaggcaacagacgggtctaacacggattggacgaaccactgaattccgcattgcagagatattgtatttaagtgcctagctcgatacaataaacgccatttgaccattcaccacattggtgtgcacctgggttgatggccggaccgttgattccctgacgactacgagcacatgcatgaagcagaaggcttcatttggtgaccccgacgtgatcgttagggaatagtggtcggccacaggcggcgtggcgatcctgtcctcatccgtctcgcttattcggggagcggacgatgaccctagtagagggggctgcggcttaggagggcagaagctgagtggcgtcggagggagccctactgcagggggccaacataccctaccgagaactcagagagtcgttggaagacgggaaggaagcccgacgactgagcggtccaccccaggcgtgattccggttgctctgcgtgattccggtcgcccggtggatcaagcatggaagccgtcataaaggtgatttcgtccgcgtgtaagacctattgcgggaaaacctctccttctaagaaggaaataggggctatgttgtccctgttacaaaaggaagggttgcttacgtccccctcagacttatattccccggggtcctgggatcccattaccgcggcgctctctcagcgggctatggtacttggaaaatcgggagagttaaaaacctggggattggttttgggggcattgaaggcggctcgagggaagatcccatatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgggctcgatcccctcgcgagttggttcagctgctgcctgaggctggacgacctcgcggagttctaccggcagtgcaaatccgtcggcatccaggaaaccagcagcggctatccgcgcatccatgcccccgaactgcaggagtggggaggcacgatggccgctttggtcaacagcgtgccgcagatcctctagaggatcgatccccctttgtatagtccatcctacaagagcaaaaacttgtaagcatttcaggtagcaaagtaatgaaaccacaaaatgctgaaaatgctccacagttgaatgcacagatatttatgttctgtgtttattcctgaagtgtaaggaatgtaaggaaatcatagattagttgtttctttcctttttgcagggcccagaccaccataagcttgggatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctttcaccatgggcgtgcacgagtgccctgcttggctgtggctgctcttgagcctgctcagcctgcctctgggcctgcctgtgctgggcgctcctccaaggctgatctgcgatagcagggtgctggagaggtacctgctggaggctaaggaggctgagaacatcaccaccggctgcgctgagcactgcagcctgaacgagaacatcaccgtgcctgataccaaggtgaacttttacgcttggaagaggatggaggtgggccagcaggctgtggaggtgtggcagggcctggctctgctgagcgaggctgtgctgaggggccaggctctgctggtgaacagctctcagccttgggagcctctgcagctgcacgtggataaggctgtgagcggcctgagaagcctgaccaccctgctgagggctctgggcgctcagaaggaggctatcagccctccagatgctgcaagcgctgcccctctgaggaccatcaccgctgatacctttaggaagctgtttagggtgtacagcaactttctgaggggcaagctgaagctgtacaccggcgaggcttgcaggaccggcgattagaaagtctagtatggggattggtggcgacgactcctggagcccgtcagtatcggcggaattccagctgagcgccggtcgctaccattaccagttggtctggtgtcaaaaataataataaccgggcaggccatgtctgcccgtatttcgcgtaaggaaatccattatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcggcggatcccccgggctgcagaaccgagcggctattgacttcttgctcctagctcacggccatggctgtgaggacattgccggaatgtgttgtttcaatctgagtgatcacagtgagtctatacagaagaagttccagctaatgaaggaacatgtcaataagatcggcgtgaacaacgacccaatcggaagttggctgcgaggattattcggaggaataggagaatgggccgtacacttgctgaaaggactgcttttggggcttgtagttatcttgttgctagtagtatgcttgccttgccttttgcaatgtgtatctagtagtattcgaaagatgattgataattcactcggctatcgcgaggaatataaaaaaattacaggaggcttataagcagcccgaaagaagagcgtaggcgagttcttgtattccgtgtgatagctggttggattggtaattgatcggctggcacgcggaatataggaggtcgctgaatagtaaacttgtagacttggctacagcatagagtatcttctgtagctctgatgactgctaggaaataatgctacggataatgtggggagggcaaggcttgcgaatcgggttgtaacgggcaaggcttgactgaggggacaatagcatgtttaggcgaaaagcggggcttcggttgtacgcggttaggagtcccctcaggatatagtagtttcgcttttgcatagggagggggaaatgtagtcttatgcaatactcttgtagtcttgcaacatgcttatgtaacgatgagttagcaacatgccttataaggagagaaaaagcaccgtgcatgccgattggtgggagtaaggtggtatgatcgtggtatgatcgtgccttgttaggaaggcaacagacgggtctaacacggattggacgaaccactgaattccgcattgcagagatattgtatttaagtgcctagctcgatacaataaacgccatttgaccattcaccacattggtgtgcacctgggttgatggccggaccgttgattccctgacgactacgagcacatgcatgaagcagaaggcttcatttggtgaccccgacgtgatcgttagggaatagtggtcggccacaggcggcgtggcgatcctgtcctcatccgtctcgcttattcggggagcggacgatgaccctagtagagggggctgcggcttaggagggcagaagctgagtggcgtcggagggagccctactgcagggggccaacataccctaccgagaactcagagagtcgttggaagacgggaaggaagcccgacgactgagcggtccaccccaggcgtgattccggttgctctgcgtgattccggtcgcccggtggatcaagcatggaagccgtcataaaggtgatttcgtccgcgtgtaagacctattgcgggaaaacctctccttctaagaaggaaataggggctatgttgtccctgttacaaaaggaagggttgcttacgtccccctcagacttatattccccggggtcctgggatcccattaccgcggcgctctctcagcgggctatggtacttggaaaatcgggagagttaaaaacctggggattggttttgggggcattgaaggcggctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggccgctctagaactagtggatcccccgggctgcagaaccgagcggctattgacttcttgctcctagctcacggccatggctgtgaggacattgccggaatgtgttgtttcaatctgagtgatcacagtgagtctatacagaagaagttccagctaatgaaggaacatgtcaataagatcggcgtgaacaacgacccaatcggaagttggctgcgaggattattcggaggaataggagaatgggccgtacacttgctgaaaggactgcttttggggcttgtagttatcttgttgctagtagtatgcttgccttgccttttgcaatgtgtatctagtagtattcgaaagatgattgataattcactcggctatcgcgaggaatataaaaaaattacaggaggcttataagcagcccgaaagaagagcgtaggcgagttcttgtattccgtgtgatagctggttggattggtaattgatcggctggcacgcggaatataggaggtcgctgaatagtaaacttgtagacttggctacagcatagagtatcttctgtagctctgatgactgctaggaaataatgctacggataatgtggggagggcaaggcttgcgaatcgggttgtaacgggcaaggcttgactgaggggacaatagcatgtttaggcgaaaagcggggcttcggttgtacgcggttaggagtcccctcaggatatagtagtttcgcttttgcatagggaggggga

SEQ ID NO: 19 - DNA, 9597 bp, Artificial Sequence
  pALV-SIN-4.2-Lys-IFNa-2B
gatcccccgtgctgcagaaccgagcggctattgacttcttgctcctagctcacggccatggctgtgaggacattgcgggaatgtgttgtttcaatctgagtgatcacagtgagtctatacagaagaagttccagctaatgaaggaacatgtcaataagatcggcgtgaacaacgacccaatcggaagttggctgcgaggattattcggaggaataggagaatgggccgtacacttgctgaaaggactgcttttggggcttgtagttatcttgttgctagtagtatgcttgccttgccttttgcaatgtgtatctagtagtattcgaaagatgattgataattcactcggctatcgcgaggaatataaaaaaattacaggaggcttataagcagcccgaaagaagagcgtaggcgagttcttgtattccgtgtgatagctggttggattggtaattgatcggctggcacgcggaatataggaggtcgctgaatagtaaacttgtagacttggctacagcatagagtatcttctgtagctctgatgactgctaggaaataatgctacggataatgtggggagggcaaggcttgcgaatcgggttgtaacgggcaaggcttgactgaggggacaatagcatgtttaggcgaaaagcggggcttcggttgtacgcggttaggagtcccctcaggatatagtagtttcgcttttgcatagggagggggacggattggacgaaccactgaattccgcattgcagagatattgtatttaagtgcctagctcgatacaataaacgccatttgaccattcaccacattggtgtgcacctgggttgatggccggaccgttgattccctgrcgactacgagcacatgcatgaagcagaaggcttcatttggtgaccccgacgtgatcgttagggaatacgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcattggtaattgatcggctggcacgcggaatataggaggtcgctgaatagtaaacttgtagacttggctacagcatagagtatcttctgtagctctgatgactgctaggaaataatgctacggataatgtggggagggcaaggcttgcgaatcgggttgtaacgggcaaggcttgactgaggggacaatagcatgtttaggcgaaaagcggggcttcggttgtacgcggttaggagtcccctcaggatatagtagtttcgcttttgcatagggagggggaaatgtagtcttatgcaatactcttgtagtcttgcaacatgcttatgtaacgatgagttagcaacatgccttataaggagagaaaaagcaccgtgcatgccgattggtgggagtaaggtggtatgatcgtggtatgatcgtgccttgttaggaaggcaacagacgggtctaacacggattggacgaaccactgaattccgcattgcagagatattgtatttaagtgcctagctcgatacaataaacgccatttgaccattcaccacattggtgtgcacctgggttgatggccggaccgttgattccctgrcgactacgagcacatgcatgaagcagaaggcttcatttggtgaccccgacgtgatcgttagggaatagtggtcggccacaggcggcgtggcgatcctgtcctcatccgtctcgcttattcggggagcggacgatgaccctagtagagggggctgcggcttaggagggcagaagctgagtggcgtcggagggagccctactgcagggggccaacataccctaccgagaactcagagagtcgttggaagacgggaaggaagcccgacgactgagcggtccaccccaggcgtgattccggttgctctgcgtgattccggtcgcccggtggatcaagcatggaagccgtcataaaggtgatttcgtccgcgtgtaagacctattgcgggaaaacctctccttctaagaaggaaataggggctatgttgtccctgttacaaaaggaagggttgcttacgtccccctcagacttatattccccggggtcctgggatccgataccgtccctatttttgtgtttgcttcagcagccatttaattcttcagtgtcatcttgttctgttgatgccactggaacaggattttcagcagtcttgcaaagaacatctagctgaaaactttctgccattcaatattcttaccagttcttcttgtttgaggtgagccataaattactagaacttcgtcactgacaagtttatgcattttattacttctattatgtacttactttgacataacacagacacgcacatattttgctgggatttccacagtgtctctgtgtccttcacatggttttactgtcatacttccgttataaccttggcaatctgcccagctgcccatcacaagaaaagagattccttttttattacttctcttcagccaataaacaaaatgtgagaagcccaaacaagaacttgtggggcaggctgccatcaagggagagacagctgaagggttgtgtagctcaatagaattaagaaataataaagctgtgtcagacagttttgcctgatttatacaggcacgccccaagccagagaggctgtctgccaaggccaccttgcagtccttggtttgtaagataagtcataggtaacttttctggtgaattgcgtggagaatcatgatggcagttcttgctgtttactatggtaagatgctaaaataggagacagcaaagtaacacttgctgctgtaggtgctctgctatccagacagcgatggcactcgcacaccaagatgagggatgctcccagctgacggatgctggggcagtaacagtgggtcccatgctgcctgctcattagcatcacctcagccctcaccagcccatcagaaggatcatcccaagctgaggaaagttgctcatcttcttcacatcatcaaacctttggcctgactgatgcctcccggatgcttaaatgtggtcactgacatctttatttttctatgatttcaagtcagaacctccggatcaggagggaacacatagtgggaatgtaccctcagctccaaggccagatcttccttcaatgatcatgcatgctacttaggaaggtgtgtgtgtgtgaatgtagaattgcctttgttattttttcttcctgctgtcaggaacattttgaataccagagaaaaagaaaagtgctcttcttggcatgggaggagttgtcacacttgcaaaataaaggatgcagtcccaaatgttcataatctcagggtctgaaggaggatcagaaactgtgtatacaatttcaggcttctctgaatgcagcttttgaaagctgttcctggccgaggcagtactagtcagaaccctcggaaacaggaacaaatgtcttcaaggtgcagcaggaggaaacaccttgcccatcatgaaagtgaataaccactgccgctgaaggaatccagctcctgtttgagcaggtgctgcacactcccacactgaaacaacagttcatttttataggacttccaggaaggatcttcttcttaagcttcttaattatggtacatctccagttggcagatgactatgactactgacaggagaatgaggaactagctgggaatatttctgtttgaccaccatggagtcacccatttctttactggtatttggaaataataattctgaattgcaaagcaggagttagcgaagatcttcatttcttccatgttggtgacagcacagttctggctatgaaagtctgcttacaaggaagaggataaaaatcatagggataataaatctaagtttgaagacaatgaggttttagctgcatttgacatgaagaaattgagacctctactggatagctatggtatttacgtgtctttttgcttagttacttattgaccccagctgaggtcaagtatgaactcaggtctctcgggctactggcatggattgattacatacaactgtaattttagcagtgatttagggtttatgagtacttttgcagtaaatcatagggttagtaatgttaatctcagggaaaaaaaaaaaaagccaaccctgacagacatcccagctcaggtggaaatcaaggatcacagctcagtgcggtcccagagaacacagggactcttctcttaggacctttatgtacagggcctcaagataactgatgttagtcagaagactttccattctggccacagttcagctgaggcaatcctggaattttctctccgctgcacagttccagtcatcccagtttgtacagttctggcactttttgggtcaggccgtgatccaaggagcagaagttccagctatggtcagggagtgcctgaccgtcccaactcactgcactcaaacaaaggcgaaaccacaagagtggcttttgttgaaattgcagtgtggcccagaggggctgcaccagtactggattgaccacgaggcaacattaatcctcagcaagtgcaatttgcagccattaaattgaactaactgatactacaatgcaatcagtatcaacaagtggtttggcttggaagatggagtctaggggctctacaggagtagctactctctaatggagttgcattttgaagcaggacactgtgaaaagctggcctcctaaagaggctgctaaacattagggtcaattttccagtgcactttctgaagtgtctgcagttccccatgcaaagctgcccaaacatagcacttccaattgaatacaattatatgcaggcgtactgcttcttgccagcactgtccttctcaaatgaactcaacaaacaatttcaaagtctagtagaaagtaacaagctttgaatgtcattaaaaagtatatctgctttcagtagttcagcttatttatgcccactagaaacatcttgtacaagctgaacactggggctccagattagtggtaaaacctactttatacaatcatagaatcatagaatggcctgggttggaagggaccccaaggatcatgaagatccaacacccccgccacaggcagggccaccaacctccagatctggtactagaccaggcagcccagggctccatccaacctggccatgaacacctccagggatggagcatccacaacctctctgggcagcctgtgccagcacctcaccaccctctctgtgaagaacttttccctgacatccaatctaagccttccctccttgaggttagatccactcccccttgtgctatcactgtctactcttgtaaaaagttgattctcctcctttttggaaggttgcaatgaggtctccttgcagccttcttctcttctgcaggatgaacaagcccagctccctcagcctgtctttataggagaggtgctccagccctctgatcatctttgtggccctcctctggacccgctccaagagctccacatctttcctgtactgggggccccaggcctgaatgcagtactccagatggggcctcaaaagagcagagtaaagagggacaatcaccttcctcaccctgctggccagccctcttctgatggagccctggatacaactggctttctgagctgcaacttctccttatcagttccactattaaaacaggaacaatacaacaggtgctgatggccagtgcagagtttttcacacttcttcatttcggtagatcttagatgaggaacgttgaagttgtgcttctgcgtgtgcttcttcctcctcaaatactcctgcctgatacctcaccccacctgccactgaatggctccatggccccctgcagccagggccctgatgaacccggcactgcttcagatgctgtttaatagcacagtatgaccaagttgcacctatgaatacacaaacaatgtgttgcatccttcagcacttgagaagaagagccaaatttgcattgtcaggaaatggtttagtaattctgccaattaaaacttgtttatctaccatggctgtttttatggctgttagtagtggtacactgatgatgaacaatggctatgcagtaaaatcaagactgtagatattgcaacagactataaaattcctctgtggcttagccaatgtggtacttcccacattgtataagaaatttggcaagtttagagcaatgtttgaagtgttgggaaatttctgtatactcaagagggcgtttttgacaactgtagaacagaggaatcaaaagggggtgggaggaagttaaaagaagaggcaggtgcaagagagcttgcagtcccgctgtgtgtacgacactggcaacatgaggtctttgctaatcttggtgctttgcttcctgcccctggctgccttagggtgcgatctgcctcagacccacagcctgggcagcaggaggaccctgatgctgctggctcagatgaggagaatcagcctgtttagctgcctgaaggataggcacgattttggctttcctcaagaggagtttggcaaccagtttcagaaggctgagaccatccctgtgctgcacgagatgatccagcagatctttaacctgtttagcaccaaggatagcagcgctgcttgggatgagaccctgctggataagttttacaccgagctgtaccagcagctgaacgatctggaggcttgcgtgatccagggcgtgggcgtgaccgagacccctctgatgaaggaggatagcatcctggctgtgaggaagtactttcagaggatcaccctgtacctgaaggagaagaagtacagcccctgcgcttgggaagtcgtgagggctgagatcatgaggagctttagcctgagcaccaacctgcaagagagcttgaggtctaaggagtaaaaagtctag

SEQ ID NO: 20 - DNA, 6411 bp, Artificial Sequence
  pTombak-G-CSF
cagagccccatgaagctgatggccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctagttgcctcccatctgcagagcttcctggaggtgtcgtaccgcgttctacgccaccttgcccagccctgaaaagtctagtatggggattggtggcgacgactcctggagcccgtcagtatcggcggaattccagctgagcgccggtcgctaccattaccagttggtctggtgtcaaaaataataataaccgggcaggccatgtctgcccgtatttcgcgtaaggaaatccattatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcggcggatcccccgggctgcagaaccgagcggctattgacttcttgctcctagctcacggccatggctgtgaggacattgccggaatgtgttgtttcaatctgagtgatcacagtgagtctatacagaagaagttccagctaatgaaggaacatgtcaataagatcggcgtgaacaacgacccaatcggaagttggctgcgaggattattcggaggaataggagaatgggccgtacacttgctgaaaggactgcttttggggcttgtagttatcttgttgctagtagtatgcttgccttgccttttgcaatgtgtatctagtagtattcgaaagatgattgataattcactcggctatcgcgaggaatataaaaaaattacaggaggcttataagcagcccgaaagaagagcgtaggcgagttcttgtattccgtgtgatagctggttggattggtaattgatcggctggcacgcggaatataggaggtcgctgaatagtaaacttgtagacttggctacagcatagagtatcttctgtagctctgatgactgctaggaaataatgctacggataatgtggggagggcaaggcttgcgaatcgggttgtaacgggcaaggcttgactgaggggacaatagcatgtttaggcgaaaagcggggcttcggttgtacgcggttaggagtcccctcaggatatagtagtttcgcttttgcatagggagggggaaatgtagtcttatgcaatactcttgtagtcttgcaacatgcttatgtaacgatgagttagcaacatgccttataaggagagaaaaagcaccgtgcatgccgattggtgggagtaaggtggtatgatcgtggtatgatcgtgccttgttaggaaggcaacagacgggtctaacacggattggacgaaccactgaattccgcattgcagagatattgtatttaagtgcctagctcgatacaataaacgccatttgaccattcaccacattggtgtgcacctgggttgatggccggaccgttgattccctgrcgactacgagcacatgcatgaagcagaaggcttcatttggtgaccccgacgtgatcgttagggaatagtggtcggccacaggcggcgtggcgatcctgtcctcatccgtctcgcttattcggggagcggacgatgaccctagtagagggggctgcggcttaggagggcagaagctgagtggcgtcggagggagccctactgcagggggccaacataccctaccgagaactcagagagtcgttggaagacgggaaggaagcccgacgactgagcggtccaccccaggcgtgattccggttgctctgcgtgattccggtcgcccggtggatcaagcatggaagccgtcataaaggtgatttcgtccgcgtgtaagacctattgcgggaaaacctctccttctaagaaggaaataggggctatgttgtccctgttacaaaaggaagggttgcttacgtccccctcagacttatattccccggggtcctgggatcccattaccgcggcgctctctcagcgggctatggtacttggaaaatcgggagagttaaaaacctggggattggttttgggggcattgaaggcggctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggccgcaatgtagtcttatgcaatactcttgtagtcttgcaacatgcttatgtaacgatgagttagcaacatgccttataaggagagaaaaagcaccgtgcatgccgattggtgggagtaaggtggtatgatcgtggtatgatcgtgccttgttaggaaggcaacagacgggtctaacacggattggacgaaccactgaattccgcattgcagagatattgtatttaagtgcctagctcgatacaataaacgccatttgaccattcagcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagaccggatggtgaccccgacgtgatcgttagggaatagtggtcggccacaggcggcgtggcgatcctgtcctcatccgtctcgcttattcggggagcggacgatgaccctagtagagggggctgcggcttaggagggcagaagctgagtggcgtcggagggagccctactgcagggggccaacataccctaccgagaactcagagagtcgttggaagacgggaaggaagcccgacgactgagcggtccaccccaggcgtgattccggttgctctgcgtgattccggtcgcccggtggatcaagcatggctggacctgccacc

SEQ ID NO: 21 - DNA, 10197 bp, Artificial Sequence
  pCMV-gag-pol-SRD
ggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaaccagctggggctcgaggggggatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgcctttactgagcactctttaatagtggactcttgttccaaactggaacaacactcaaccctatctcggtctattcttttgatttataagatttccatcgccatgtaaaagtgttacaattagcattaaattacttctttatatgctactattcttttggcttcgttcacggggtgggtaccgagctcgaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttggatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctggatcccgtcgacctcgagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagaccggaagcttggatcaagcatggaagccgtcataaaggtgatttcgtccgcgtgtaagacctattgcgggaaaacctctccttctaagaaggaaataggggctatgttgtccctgttacaaaaggaagggttgcttatgtctccctcagacttatattccccggggtcctgggatcccattaccgcggcgctctcccagcgggcaatgatacttggaaaatcgggagagttaaaaacctggggattggttttgggggcattgaaggcggctcgagaggaacaggttacatctgagcaagcaaagttttggttgggattagggggagggagggtctctcccccaggtccggagtgcatcgagaaaccagctacggagcggcgaatcgacaaaggggaggaagtgggcgaaacaactgtgcagcgagatgcgaagatggcgccggaggaagcggccacacctaaaaccgttggcacatcctgctatcattgcggaacagctgttggctgcaattgcgccaccgccacagcctcggcccctcctcccccttatgtggggagtggtttgtatccttccctggcgggggtgggagagcagcagggccagggagatagggcggagcagccaagggaggagccagggcacgcgggtcaggcccctgggccggccctgactgactgggcaagggtaagagaggagcttgcgagtacaggtccgcccgtggtggccatgcccgtagtgattaagacagagggacccgcctggacccctctggagccaaaattgatcacaagactggctgatacggtcaggaccaagggcttacgatccccgatcactatggcagaagtggaagcgctcatgtcctccccgttgctgccgcatgacgtcacgaatctaatgagagttattttaggacctgccccatatgccttatggatggacgcttggggagtccaactccagacggttatagcggcagccactcgcgacccccgacacccagcgaacggtcaagggcggggggaacggactaacttggatcgattaaagggcttagctgatgggatggtgggcaacccacagggtcaggccgcattattaagaccgggggaattggttgctattacggcgtcggctctccaggcgtttagagaagttgcccggctggcggaacctgcaggtccatgggcggacatcacgcagggaccatctgagtcctttgttgattttgccaatcggcttataaaggcggttgaggggtcagatctcccgcctaccgcgcgggctccggtgattattgactgctttaggcagaagtcgcagccagacattcagcagcttatacgggcagcaccctccacactgaccaccccaggagagataatcaaatatgtgctggataggcagaaaactgcccctcttacggatcaaggcatagctgcggccatgtcgtctgctatccagcccttagttatggcagtagtcaatagagagagggatggacaaactgggtcgggtggtcgtgcccgagggctctgctacacttgtggatccccgggacattatcaggcacagtgcccgaaaaaacgaaaatcaggaaacagccgtgagcgatgtcagctgtgtgacgggatgggacacaacgctaaacagtgtaggaagcgggatggcaaccagggccaacgcccaggaagaggtctctcttcggggccgtggcccggccctgagcagcctgccgtctcgttagcgatgacaatggaacataaagatcgccccttggttagggtcattctgactaacactgggagtcatccagtcaaacagcgttcggtgtatatcaccgcgctgttggactccggagcagacatcactattatttcggaggaggattggcctactgattggccggtggtggacaccgcgaacccacagatccatggcataggagggggaattcccatgcgaaaatctcgtgacatgatagagttgggggttattaaccgagacgggtccttggagcgacccctgctcctcttccccgcagtggctatggttagagggagtatcctaggaagagattgtctgcagggcctagggctccgcttgacaaatttatagggagggccactgttcttactgttgcgctacatctggctattccgctcaaatggaagccagaccacacgcctgtgtggattgaccagtggccccttcctgagggtaaacttgtagcgctaacgcaattagtggaaaaagaattgcagctaggacatatagaaccttcacttagttgttggaacacacctgtctttgtgatccggaaggcttccgggtcttatcgcttattgcatgacttgcgcgctgttaacgctaagcttgttccttttggggccgtccaacagggggcgccagttctctccgcgctcccgcgtggctggcccctgatggtcctagacctcaaggattgcttcttctctattcctcttgcggaacaagatcgcgaagcttttgcatttacgctcccctctgtgaataaccaggcccccgctcgaagattccaatggaaggtcttgccccaaggaatgacctgttctcccactatctgtcagttggtagtgggtcaggtacttgagcccttgcgactcaagcacccatctctgcgcatgttgcattatatggatgatcttttgctagccgcctcaagtcatgatgggttggaagaggcaggggaggaggttatcagtacattggaaagagccgggttcaccatttcgcctgataaggtccagagggagcccggagtacaatatcttgggtacaagttaggcagtacgtatgtagcacccgtaggcctggtagcagaacccaggatagccaccttgtgggatgttcaaaagctggtagggtcacttcagtggcttcgcccagcgttaggaatcccgccacgactgatgggccccttctatgagcagttacgagggtcagatcctaacgaggcgagggaatggaatctagacatgaaaatggcctggagggagatcgtacagcttagcaccactgctgccttggaacgatgggaccctgccctgcctttggagggagcggtcgctaggtgtgaacagggggcaataggggtcctgggacagggactgtccacacacccaaggccatgtttgtggctattttccacccaacccaccaaggcgtttactgcctggatagaagtactcacccttttgattactaagctacgcgcttcggcagtgcgaacctttggcaaggaggttgatatcctcctgttgcctgcatgctttcgggaggaccttccgctcccggaggggatcctgttagcccttagggggtttgcaggaaaaatcaggagtagtgacacgccatctatttttgacattgcgcgtccactgcatgtttctctgaaagtgagggtcaccgaccaccctgtaccgggacccactgcctttaccgacgcctcctcaagcacccataaaggggtggtagtctggagggagggcccaaggtgggagataaaagaaatagctgatctgggggcaagtgtacaacaactggaagcacgcgctgtggccatggcacttttgctgtggccgacaacgcccactaatgtagtgactgactccgcgtttgttgcgaaaatgttactcaagatgggtcaggagggagtcccatctacagcggcggcttttattttagaggatgcgttaagccaaaggtcagccatggccgccattctccatgtgcggagtcattctgaagtgccagggtttttcacagaaggaaatgacgtggcagacagccaagccacctttcaagcgtatcccttgagagaggctaaagatcttcatactgctctccatattggaccccgcgcgctatccaaagcgtgtaatatatctatgcagcaggctagggaggttgttcagacctgcccgcattgtaattcagcccctgcgttggaggccggagtaaaccctaggggtttgggacccctacagatatggcagacagactttacgcttgagcctagaatggccccccgttcctggctcgctgttactgtggataccgcctcatcagcgatagtcgtaactcagcatggccgtgtcacatcggttgctgcacaacatcattgggccacggctatcgccgttttgggaagaccaaaggccataaaaacagataatgggtcctgtttcacgtctaaatccacgcgagagtggctcgcgagatgggggatagcacacaccaccgggattccgggtaattcccagggtcaagctatggtagagcgggccaaccggctcctgaaagataagatccgtgtgcttgcggagggggacggctttatgaaaagaatccccgccagcaagcagggggaactactagccaaggcaatgtatgccctcaatcactttgagcgtggtgaaaacacaaaaacaccgattcaaaaacactggagacctaccgttcttacagaaggacccccggttaaaatacgaatagagacaggggagtgggaaaaaggatggaatgtgctggtctggggacgaggttatgcagctgtgaaaaacagggacactgataaggttatttgggtaccctctcggaaagttaaaccggatgtcacccaaaaggatgaggtgactaagaaagatgaggcgagccctctttttgcaggcatttctgactggataccctgggaagacgagcaagaaggactccaaggagaaaccgctagcaacaagcaagaaagacccggagaagacacccttgcagccaacgagagttaagc

SEQ ID NO: 22 - DNA, 10203 bp, Artificial Sequence
  pCMV-gag-pol-PRC
ggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaaccagctggggctcgaggggggatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgcctttactgagcactctttaatagtggactcttgttccaaactggaacaacactcaaccctatctcggtctattcttttgatttataagatttccatcgccatgtaaaagtgttacaattagcattaaattacttctttatatgctactattcttttggcttcgttcacggggtgggtaccgagctcgaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttggatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctggatcccgtcgacctcgagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagaccggaagcttggatcaagcatggaagccgtcataaaggtgatttcgtccgcgtgtaagacctattgcgggaaaacctctccttctaagaaggaaataggggctatgttgtccctgttacaaaaggaagggttgcttatgtctccctcagacttatattccccggggtcctgggatcccattaccgcggcgctatcccagcgggctatgatacttgggaaatcgggagagttaaaaacctggggattggttttgggggcattgaaggcggctcgagaggaacaggttacatctgagcaagcaaagttttggttgggattagggggagggagggtctctcccccaggtccggagtgcatcgagaaaccagcaacggagcggcgaatcgacaaaggggaggaagtgggagaaacaactgtgcagcgagatgcgaagatggcgccggaggaaacggccacacctaaaaccgttggcacatcctgctatcattgcggaacagctattggctgtaattgcgccacagcctcggctcctcctcctccttatgtggggagtggtttgtatccttccctggcgggggtgggagagcagcagggccaggggggtgacacacctccgggggcggaacagtcaagggcggagccagggcatgcgggtcaggctcctgggccggccctgactgactgggcaagggtcagggaggagcttgcgagtactggtccgcccgtggtggccatgcctgtagtgattaagacagagggacccgcttggacccctctggagccaaaattgatcacaagactggctgatacggtcaggaccaagggcttacgatccccgattactatggcagaagtggaagcgcttatgtcctccccgctgctgccgcatgacgtcacgaatctaatgagagttattttagggcctgccccatatgccttatggatggacgcttggggagtccaactccagacagttatagcggcagccactcgcgacccccgacacccagcgaacggtcaagggcggggggaacggactaatttgaatcgcttaaagggcttagctgatgggatggtgggcaacccacagggtcaggccgcattattaagaccgggggaattggttgctattacggcgtcggctctccaggcgtttagagaggttgcccggctggcggaacctgcaggtccatgggcggacatcatgcagggaccatctgagtcctttgttgattttgccaatcggcttataaaggcggttgaggggtcagatctcccgccttccgcgcgggctccggtgatcattgactgctttaggcagaagtcacagccagatattcagcagcttatacggacagcaccctccacgctgaccaccccaggagagataattaaatatgtgctagacaggcagaagactgcccctcttacggatcaaggcatagctgcggccatgtcgtctgctatccagcccttaattatggcagtagtcaatagagagagggatggacaaactgggtcgggtggtcgtgcccgagggctctgctacacttgtggatccccgggacattatcaggcgcagtgcccgaaaaaacggaagtcaggaaacagccgtgagcgatgtcagttgtgtaacgggatgggacacaacgctaaacagtgtaggaagcgggatggcaaccagggccaacgcccaggaaaaggtctctcttcggggccgtggcccggccctgagccacctgccgtctcgttagcgatgacaatggaacataaagatcgccccttggttagggtcattctgactaacactgggagtcatccggtcaaacagcgttcggtgtatatcaccgcgctgttggactctggagcggacatcactattatttcagaggaggattggcccaccgattggccagtgatggaggccgcgaacccgcagatccatgggataggagggggaattcccatgcgaaaatctcgtgacatgatagagttgggggttattaaccgagacgggtctttggagcgacccctgctcctcttccccgcagtagctatggttagagggagtatcctaggaagagattgtctgcagggcctagggctccgcttgacaaatttatagggagggccactgttctcactgttgcgctacatctggctattccgctcaaatggaagccagaccacacgcctgtgtggattgaccagtggcccctccctgaaggtaaacttgtagcgctaacgcaattagtggaaaaagaattacagttaggacatatagaaccttcacttagttgttggaacacacctgtcttcgtgatccggaaggcttccgggtcttaccgcttactgcatgatttgcgcgctgttaacgccaagcttgttccttttggggccgtccaacagggggcgccagttctctccgcgctcccgcgtggctggcccctgatggtcttagacctcaaggattgcttcttttctatccctcttgcggaacaagatcgcgaagcttttgcatttacgctcccctctgtgaataaccaggcccccgctcgaagattccaatggaaggtcttgccccaagggatgacctgttctcccactatctgtcagttggtagtgggtcaggtacttgagcccttgcgactcaagcacccatctctgtgcatgttgcattatatggatgatcttttgctagccgcctcaagtcacgatgggttggaagcggcaggggaggaggttatcagtacattggaaagagccgggttcactatttcgcctgataaggtccagagggagcccggagtacaatatcttgggtacaagttaggcagtacgtatgtagcacccgtaggcctggtagcagaacccaggatagccaccttgtgggatgttcaaaagctggtggggtcacttcagtggcttcgcccagcgttaggaatcccgccacgactgatgggccccttctatgagcagttacgagggtcagatcctaacgaggcgagggaatggaatctagacatgaaaatggcctggagagagatcgtacggcttagcaccactgctgccttggaacgatgggaccctgccctgcctctggaaggagcggtcgctagatgtgaacagggggcaataggggttttgggacagggactgtccacacacccaaggccatgcttgtggttattctccacccaacccaccaaggcgtttactgcttggttagaagtgctcacccttttgattactaagctacgtgcttcggcagtgcgaacctttggcaaggaggtcgatatcctcctgttgcctgcatgctttcgggaggaccttccgctcccagaggggatcctgttagcccttaaggggtttgcaggaaaaatcaggagtagtgacacgccatctatttttgacattgcgcgtccactgcatgtttctctgaaagtgagggttaccgaccaccctgtgccgggacccactgtctttactgacgcctcctcaagcacccataagggggtggtagtctggagggagggcccaaggtgggagataaaagaaatagctgatttgggggcaagtgtacaacaactggaagcacgcgctgtggccatggcacttctgctgtggccgacaacgcccactaatgtagtgactgactccgcgtttgttgcgaaaatgttactcaagatgggacaggagggagtcccgtctacagcggcggcttttattttagaggatgcgttaagccaaaggtcagccatggccgccgttctccacgtgcggagtcattctgaagtgccagggtttttcacagaaggaaatgacgtggcagatagccaagccaccttccaagcgtatcccttgagagaggctaaagatcttcataccgctctccatattggaccccgcgcgctatccaaagcgtgtaatatatctatgcagcaggctagggaggttgttcagacctgcccgcattgtaattcagcccctgcgttggaggccggagtaaaccctaggggtttgggacccctacagatatggcagacagactttacgcttgagcctagaatggccccccgttcctggctcgctgttactgtggataccgcctcatcagcgatagtcgtaactcagcatggccgtgtcacatcggttgctgtacaacatcattgggccacggctatcgccgttttgggaagaccaaaggccataaaaacagataatgggtcctgcttcacgtctaaatccacgcgagagtggctcgcgagatgggggatagcacacaccaccgggattccgggtaattcccagggtcaagctatggtagagcgggccaaccggctcctgaaagataggatccgtgtgcttgcggagggggacggctttatgaaaagaatccccaccagcaaacagggggaactattagccaaggcaatgtatgccctcaatcactttgagcgtggtgaaaacacgaaaacaccgatacaaaaacactggagacctaccgttcttacagaaggacccccggttaaaatacgaatagagacaggggagtgggaaaaaggatggaacgtgctggtctggggacgaggttatgccgctgtgaaaaacagggacactgataaggttatttgggtaccctctcgaaaagttaaaccggacatcacccaaaaggatgaggtgactaagaaagatgaggcgagccctctttttgcaggcatttctgactggataccctgggaagacgagcaagaaggactccaaggagaaaccgctagcaacaagcaagaaagacccggagaagacacccttgctgccaacgagagttaagc

SEQ ID NO: 23 - DNA, 10197 bp, Artificial Sequence
  pCMV-gag-SRD-pol-PRC
aacgccaagcttgttccttttggggccgtccaacagggggcgccagttctctccgcgctcccgcgtggctggcccctgatggtcttagacctcaaggattgcttcttttctatccctcttgcggaacaagatcgcgaagcttttgcatttacgctcccctctgtgaataaccaggcccccgctcgaagattccaatggaaggtcttgccccaagggatgacctgttctcccactatctgtcagttggtagtgggtcaggtacttgagcccttgcgactcaagcacccatctctgtgcatgttgcattatatggatgatcttttgctagccgcctcaagtcacgatgggttggaagcggcaggggaggaggttatcagtacattggaaagagccgggttcactatttcgcctgataaggtccagagggagcccggagtacaatatcttgggtacaagttaggcagtacgtatgtagcacccgtaggcctggtagcagaacccaggatagccaccttgtgggatgttcaaaagctggtggggtcacttcagtggcttcgcccagcgttaggaatcccgccacgactgatgggccccttctatgagcagttacgagggtcagatcctaacgaggcgagggaatggaatctagacatgaaaatggcctggagagagatcgtacggcttagcaccactgctgccttggaacgatgggaccctgccctgcctctggaaggagcggtcgctagatgtgaacagggggcaataggggttttgggacagggactgtccacacacccaaggccatgcttgtggttattctccacccaacccaccaaggcgtttactgcttggttagaagtgctcacccttttgattactaagctacgtgcttcggcagtgcgaacctttggcaaggaggtcgatatcctcctgttgcctgcatgctttcgggaggaccttccgctcccagaggggatcctgttagcccttaaggggtttgcaggaaaaatcaggagtagtgacacgccatctatttttgacattgcgcgtccactgcatgtttctctgaaagtgagggttaccgaccaccctgtgccgggacccactgtctttactgacgcctcctcaagcacccataagggggtggtagtctggagggagggcccaaggtgggagataaaagaaatagctgatttgggggcaagtgtacaacaactggaagcacgcgctgtggccatggcacttctgctgtggccgacaacgcccactaatgtagtgactgactccgcgtttgttgcgaaaatgttactcaagatgggacaggagggagtcccgtctacagcggcggcttttattttagaggatgcgttaagccaaaggtcagccatggccgccgttctccacgtgcggagtcattctgaagtgccagggtttttcacagaaggaaatgacgtggcagatagccaagccaccttccaagcgtatcccttgagagaggctaaagatcttcataccgctctccatattggaccccgcgcgctatccaaagcgtgtaatatatctatgcagcaggctagggaggttgttcagacctgcccgcattgtaattcagcccctgcgttggaggccggagtaaaccctaggggtttgggacccctacagatatggcagacagactttacgcttgagcctagaatggccccccgttcctggctcgctgttactgtggataccgcctcatcagcgatagtcgtaactcagcatggccgtgtcacatcggttgctgtacaacatcattgggccacggctatcgccgttttgggaagaccaaaggccataaaaacagataatgggtcctgcttcacgtctaaatccacgcgagagtggctcgcgagatgggggatagcacacaccaccgggattccgggtaattcccagggtcaagctatggtagagcgggccaaccggctcctgaaagataggatccgtgtgcttgcggagggggacggctttatgaaaagaatccccaccagcaaacagggggaactattagccaaggcaatgtatgccctcaatcactttgagcgtggtgaaaacacgaaaacaccgatacaaaaacactggagacctaccgttcttacagaaggacccccggttaaaatacgaatagagacaggggagtgggaaaaaggatggaacgtgctggtctggggacgaggttatgccgctgtgaaaaacagggacactgataaggttatttgggtaccctctcgaaaagttaaaccggacatcacccaaaaggatgaggtgactaagaaagatgaggcgagccctctttttgcaggcatttctgactggataccctgggaagacgagcaagaaggactccaaggagaaaccgctagcaacaagcaagaaagacccggagaagacacccttgctgccaacgagagttaagcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaaccagctggggctcgaggggggatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgcctttactgagcactctttaatagtggactcttgttccaaactggaacaacactcaaccctatctcggtctattcttttgatttataagatttccatcgccatgtaaaagtgttacaattagcattaaattacttctttatatgctactattcttttggcttcgttcacggggtgggtaccgagctcgaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttggatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctggatcccgtcgacctcgagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagaccggaagcttggatcaagcatggaagccgtcataaaggtgatttcgtccgcgtgtaagacctattgcgggaaaacctctccttctaagaaggaaataggggctatgttgtccctgttacaaaaggaagggttgcttatgtctccctcagacttatattccccggggtcctgggatcccattaccgcggcgctctcccagcgggcaatgatacttggaaaatcgggagagttaaaaacctggggattggttttgggggcattgaaggcggctcgagaggaacaggttacatctgagcaagcaaagttttggttgggattagggggagggagggtctctcccccaggtccggagtgcatcgagaaaccagctacggagcggcgaatcgacaaaggggaggaagtgggcgaaacaactgtgcagcgagatgcgaagatggcgccggaggaagcggccacacctaaaaccgttggcacatcctgctatcattgcggaacagctgttggctgcaattgcgccaccgccacagcctcggcccctcctcccccttatgtggggagtggtttgtatccttccctggcgggggtgggagagcagcagggccagggagatagggcggagcagccaagggaggagccagggcacgcgggtcaggcccctgggccggccctgactgactgggcaagggtaagagaggagcttgcgagtacaggtccgcccgtggtggccatgcccgtagtgattaagacagagggacccgcctggacccctctggagccaaaattgatcacaagactggctgatacggtcaggaccaagggcttacgatccccgatcactatggcagaagtggaagcgctcatgtcctccccgttgctgccgcatgacgtcacgaatctaatgagagttattttaggacctgccccatatgccttatggatggacgcttggggagtccaactccagacggttatagcggcagccactcgcgacccccgacacccagcgaacggtcaagggcggggggaacggactaacttggatcgattaaagggcttagctgatgggatggtgggcaacccacagggtcaggccgcattattaagaccgggggaattggttgctattacggcgtcggctctccaggcgtttagagaagttgcccggctggcggaacctgcaggtccatgggcggacatcacgcagggaccatctgagtcctttgttgattttgccaatcggcttataaaggcggttgaggggtcagatctcccgcctaccgcgcgggctccggtgattattgactgctttaggcagaagtcgcagccagacattcagcagcttatacgggcagcaccctccacactgaccaccccaggagagataatcaaatatgtgctggataggcagaaaactgcccctcttacggatcaaggcatagctgcggccatgtcgtctgctatccagcccttagttatggcagtagtcaatagagagagggatggacaaactgggtcgggtggtcgtgcccgagggctctgctacacttgtggatccccgggacattatcaggcacagtgcccgaaaaaacgaaaatcaggaaacagccgtgagcgatgtcagctgtgtgacgggatgggacacaacgctaaacagtgtaggaagcgggatggcaaccagggccaacgcccaggaagaggtctctcttcggggccgtggcccggccctgagcagcctgccgtctcgttagcgatgacaatggaacataaagatcgccccttggttagggtcattctgactaacactgggagtcatccagtcaaacagcgttcggtgtatatcaccgcgctgttggactccggagcagacatcactattatttcggaggaggattggcctactgattggccggtggtggacaccgcgaacccacagatccatggcataggagggggaattcccatgcgaaaatctcgtgacatgatagagttgggggttattaaccgagacgggtccttggagcgacccctgctcctcttccccgcagtggctatggttagagggagtatcctaggaagagattgtctgcagggcctagggctccgcttgacaaatttatagggagggccactgttcttactgttgcgctacatctggctattccgctcaaatggaagccagaccacacgcctgtgtggattgaccagtggccccttcctgagggtaaacttgtagcgctaacgcaattagtggaaaaagaattgcagctaggacatatagaaccttcacttagttgttggaacacacctgtctttgtgatccggaaggcttccgggtcttatcgcttattgcatgacttgcgcgctgtt

SEQ ID NO: 24 - DNA, 10149 bp, Artificial Sequence
  p407-gag-SRD-pol-PRC
gtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagaccggaagcttggatcaagcatggaagccgtcataaaggtgatttcgtccgcgtgtaagacctattgcgggaaaacctctccttctaagaaggaaataggggctatgttgtccctgttacaaaaggaagggttgcttatgtctccctcagacttatattccccggggtcctgggatcccattaccgcggcgctctcccagcgggcaatgatacttggaaaatcgggagagttaaaaacctggggattggttttgggggcattgaaggcggctcgagaggaacaggttacatctgagcaagcaaagttttggttgggattagggggagggagggtctctcccccaggtccggagtgcatcgagaaaccagctacggagcggcgaatcgacaaaggggaggaagtgggcgaaacaactgtgcagcgagatgcgaagatggcgccggaggaagcggccacacctaaaaccgttggcacatcctgctatcattgcggaacagctgttggctgcaattgcgccaccgccacagcctcggcccctcctcccccttatgtggggagtggtttgtatccttccctggcgggggtgggagagcagcagggccagggagatagggcggagcagccaagggaggagccagggcacgcgggtcaggcccctgggccggccctgactgactgggcaagggtaagagaggagcttgcgagtacaggtccgcccgtggtggccatgcccgtagtgattaagacagagggacccgcctggacccctctggagccaaaattgatcacaagactggctgatacggtcaggaccaagggcttacgatccccgatcactatggcagaagtggaagcgctcatgtcctccccgttgctgccgcatgacgtcacgaatctaatgagagttattttaggacctgccccatatgccttatggatggacgcttggggagtccaactccagacggttatagcggcagccactcgcgacccccgacacccagcgaacggtcaagggcggggggaacggactaacttggatcgattaaagggcttagctgatgggatggtgggcaacccacagggtcaggccgcattattaagaccgggggaattggttgctattacggcgtcggctctccaggcgtttagagaagttgcccggctggcggaacctgcaggtccatgggcggacatcacgcagggaccatctgagtcctttgttgattttgccaatcggcttataaaggcggttgaggggtcagatctcccgcctaccgcgcgggctccggtgattattgactgctttaggcagaagtcgcagccagacattcagcagcttatacgggcagcaccctccacactgaccaccccaggagagataatcaaatatgtgctggataggcagaaaactgcccctcttacggatcaaggcatagctgcggccatgtcgtctgctatccagcccttagttatggcagtagtcaatagagagagggatggacaaactgggtcgggtggtcgtgcccgagggctctgctacacttgtggatccccgggacattatcaggcacagtgcccgaaaaaacgaaaatcaggaaacagccgtgagcgatgtcagctgtgtgacgggatgggacacaacgctaaacagtgtaggaagcgggatggcaaccagggccaacgcccaggaagaggtctctcttcggggccgtggcccggccctgagcagcctgccgtctcgttagcgatgacaatggaacataaagatcgccccttggttagggtcattctgactaacactgggagtcatccagtcaaacagcgttcggtgtatatcaccgcgctgttggactccggagcagacatcactattatttcggaggaggattggcctactgattggccggtggtggacaccgcgaacccacagatccatggcataggagggggaattcccatgcgaaaatctcgtgacatgatagagttgggggttattaaccgagacgggtccttggagcgacccctgctcctcttccccgcagtggctatggttagagggagtatcctaggaagagattgtctgcagggcctagggctccgcttgacaaatttatagggagggccactgttcttactgttgcgctacatctggctattccgctcaaatggaagccagaccacacgcctgtgtggattgaccagtggccccttcctgagggtaaacttgtagcgctaacgcaattagtggaaaaagaattgcagctaggacatatagaaccttcacttagttgttggaacacacctgtctttgtgatccggaaggcttccgggtcttatcgcttattgcatgacttgcgcgctgttaacgccaagcttgttccttttggggccgtccaacagggggcgccagttctctccgcgctcccgcgtggctggcccctgatggtcttagacctcaaggattgcttcttttctatccctcttgcggaacaagatcgcgaagcttttgcatttacgctcccctctgtgaataaccaggcccccgctcgaagattccaatggaaggtcttgccccaagggatgacctgttctcccactatctgtcagttggtagtgggtcaggtacttgagcccttgcgactcaagcacccatctctgtgcatgttgcattatatggatgatcttttgctagccgcctcaagtcacgatgggttggaagcggcaggggaggaggttatcagtacattggaaagagccgggttcactatttcgcctgataaggtccagagggagcccggagtacaatatcttgggtacaagttaggcagtacgtatgtagcacccgtaggcctggtagcagaacccaggatagccaccttgtgggatgttcaaaagctggtggggtcacttcagtggcttcgcccagcgttaggaatcccgccacgactgatgggccccttctatgagcagttacgagggtcagatcctaacgaggcgagggaatggaatctagacatgaaaatggcctggagagagatcgtacggcttagcaccactgctgccttggaacgatgggaccctgccctgcctctggaaggagcggtcgctagatgtgaacagggggcaataggggttttgggacagggactgtccacacacccaaggccatgcttgtggttattctccacccaacccaccaaggcgtttactgcttggttagaagtgctcacccttttgattactaagctacgtgcttcggcagtgcgaacctttggcaaggaggtcgatatcctcctgttgcctgcatgctttcgggaggaccttccgctcccagaggggatcctgttagcccttaaggggtttgcaggaaaaatcaggagtagtgacacgccatctatttttgacattgcgcgtccactgcatgtttctctgaaagtgagggttaccgaccaccctgtgccgggacccactgtctttactgacgcctcctcaagcacccataagggggtggtagtctggagggagggcccaaggtgggagataaaagaaatagctgatttgggggcaagtgtacaacaactggaagcacgcgctgtggccatggcacttctgctgtggccgacaacgcccactaatgtagtgactgactccgcgtttgttgcgaaaatgttactcaagatgggacaggagggagtcccgtctacagcggcggcttttattttagaggatgcgttaagccaaaggtcagccatggccgccgttctccacgtgcggagtcattctgaagtgccagggtttttcacagaaggaaatgacgtggcagatagccaagccaccttccaagcgtatcccttgagagaggctaaagatcttcataccgctctccatattggaccccgcgcgctatccaaagcgtgtaatatatctatgcagcaggctagggaggttgttcagacctgcccgcattgtaattcagcccctgcgttggaggccggagtaaaccctaggggtttgggacccctacagatatggcagacagactttacgcttgagcctagaatggccccccgttcctggctcgctgttactgtggataccgcctcatcagcgatagtcgtaactcagcatggccgtgtcacatcggttgctgtacaacatcattgggccacggctatcgccgttttgggaagaccaaaggccataaaaacagataatgggtcctgcttcacgtctaaatccacgcgagagtggctcgcgagatgggggatagcacacaccaccgggattccgggtaattcccagggtcaagctatggtagagcgggccaaccggctcctgaaagataggatccgtgtgcttgcggagggggacggctttatgaaaagaatccccaccagcaaacagggggaactattagccaaggcaatgtatgccctcaatcactttgagcgtggtgaaaacacgaaaacaccgatacaaaaacactggagacctaccgttcttacagaaggacccccggttaaaatacgaatagagacaggggagtgggaaaaaggatggaacgtgctggtctggggacgaggttatgccgctgtgaaaaacagggacactgataaggttatttgggtaccctctcgaaaagttaaaccggacatcacccaaaaggatgaggtgactaagaaagatgaggcgagccctctttttgcaggcatttctgactggataccctgggaagacgagcaagaaggactccaaggagaaaccgctagcaacaagcaagaaagacccggagaagacacccttgctgccaacgagagttaagcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaaccagctggggctcgaggggggatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgcctttactgagcactctttaatagtggactcttgttccaaactggaacaacactcaaccctatctcggtctattcttttgatttataagatttccatcgccatgtaaaagtgttacaattagcattaaattacttctttatatgctactattcttttggcttcgttcacggggtgggtaccgagctcgaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttggatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctggatcccgtcgacctcgagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtaatgtagtcttatgcaatactcttgtagtcttgcaacatgcttatgtaacgatgagttagcaacatgccttataaggagagaaaaagcaccgtgcatgccgattggtgggagtaaggtggtatgatcgtggtatgatcgtgccttgttaggaaggcaacagacgggtctaacacggattggacgaaccactgaattccgcattgcagagatattgtatttaagtgcctagctcgatacaataaacgccatttgaccattcagcccagtacatgaccttatgggactttcctacttggcagtacatctac

SEQ ID NO: 25 - DNA, 10431 bp, Artificial Sequence
  pLTRrev-CMV-gag-SRD-pol-PRC
ggcgctctcccagcgggcaatgatacttggaaaatcgggagagttaaaaacctggggattggttttgggggcattgaaggcggctcgagaggaacaggttacatctgagcaagcaaagttttggttgggattagggggagggagggtctctcccccaggtccggagtgcatcgagaaaccagctacggagcggcgaatcgacaaaggggaggaagtgggcgaaacaactgtgcagcgagatgcgaagatggcgccggaggaagcggccacacctaaaaccgttggcacatcctgctatcattgcggaacagctgttggctgcaattgcgccaccgccacagcctcggcccctcctcccccttatgtggggagtggtttgtatccttccctggcgggggtgggagagcagcagggccagggagatagggcggagcagccaagggaggagccagggcacgcgggtcaggcccctgggccggccctgactgactgggcaagggtaagagaggagcttgcgagtacaggtccgcccgtggtggccatgcccgtagtgattaagacagagggacccgcctggacccctctggagccaaaattgatcacaagactggctgatacggtcaggaccaagggcttacgatccccgatcactatggcagaagtggaagcgctcatgtcctccccgttgctgccgcatgacgtcacgaatctaatgagagttattttaggacctgccccatatgccttatggatggacgcttggggagtccaactccagacggttatagcggcagccactcgcgacccccgacacccagcgaacggtcaagggcggggggaacggactaacttggatcgattaaagggcttagctgatgggatggtgggcaacccacagggtcaggccgcattattaagaccgggggaattggttgctattacggcgtcggctctccaggcgtttagagaagttgcccggctggcggaacctgcaggtccatgggcggacatcacgcagggaccatctgagtcctttgttgattttgccaatcggcttataaaggcggttgaggggtcagatctcccgcctaccgcgcgggctccggtgattattgactgctttaggcagaagtcgcagccagacattcagcagcttatacgggcagcaccctccacactgaccaccccaggagagataatcaaatatgtgctggataggcagaaaactgcccctcttacggatcaaggcatagctgcggccatgtcgtctgctatccagcccttagttatggcagtagtcaatagagagagggatggacaaactgggtcgggtggtcgtgcccgagggctctgctacacttgtggatccccgggacattatcaggcacagtgcccgaaaaaacgaaaatcaggaaacagccgtgagcgatgtcagctgtgtgacgggatgggacacaacgctaaacagtgtaggaagcgggatggcaaccagggccaacgcccaggaagaggtctctcttcggggccgtggcccggccctgagcagcctgccgtctcgttagcgatgacaatggaacataaagatcgccccttggttagggtcattctgactaacactgggagtcatccagtcaaacagcgttcggtgtatatcaccgcgctgttggactccggagcagacatcactattatttcggaggaggattggcctactgattggccggtggtggacaccgcgaacccacagatccatggcataggagggggaattcccatgcgaaaatctcgtgacatgatagagttgggggttattaaccgagacgggtccttggagcgacccctgctcctcttccccgcagtggctatggttagagggagtatcctaggaagagattgtctgcagggcctagggctccgcttgacaaatttatagggagggccactgttcttactgttgcgctacatctggctattccgctcaaatggaagccagaccacacgcctgtgtggattgaccagtggccccttcctgagggtaaacttgtagcgctaacgcaattagtggaaaaagaattgcagctaggacatatagaaccttcacttagttgttggaacacacctgtctttgtgatccggaaggcttccgggtcttatcgcttattgcatgacttgcgcgctgttaacgccaagcttgttccttttggggccgtccaacagggggcgccagttctctccgcgctcccgcgtggctggcccctgatggtcttagacctcaaggattgcttcttttctatccctcttgcggaacaagatcgcgaagcttttgcatttacgctcccctctgtgaataaccaggcccccgctcgaagattccaatggaaggtcttgccccaagggatgacctgttctcccactatctgtcagttggtagtgggtcaggtacttgagcccttgcgactcaagcacccatctctgtgcatgttgcattatatggatgatcttttgctagccgcctcaagtcacgatgggttggaagcggcaggggaggaggttatcagtacattggaaagagccgggttcactatttcgcctgataaggtccagagggagcccggagtacaatatcttgggtacaagttaggcagtacgtatgtagcacccgtaggcctggtagcagaacccaggatagccaccttgtgggatgttcaaaagctggtggggtcacttcagtggcttcgcccagcgttaggaatcccgccacgactgatgggccccttctatgagcagttacgagggtcagatcctaacgaggcgagggaatggaatctagacatgaaaatggcctggagagagatcgtacggcttagcaccactgctgccttggaacgatgggaccctgccctgcctctggaaggagcggtcgctagatgtgaacagggggcaataggggttttgggacagggactgtccacacacccaaggccatgcttgtggttattctccacccaacccaccaaggcgtttactgcttggttagaagtgctcacccttttgattactaagctacgtgcttcggcagtgcgaacctttggcaaggaggtcgatatcctcctgttgcctgcatgctttcgggaggaccttccgctcccagaggggatcctgttagcccttaaggggtttgcaggaaaaatcaggagtagtgacacgccatctatttttgacattgcgcgtccactgcatgtttctctgaaagtgagggttaccgaccaccctgtgccgggacccactgtctttactgacgcctcctcaagcacccataagggggtggtagtctggagggagggcccaaggtgggagataaaagaaatagctgatttgggggcaagtgtacaacaactggaagcacgcgctgtggccatggcacttctgctgtggccgacaacgcccactaatgtagtgactgactccgcgtttgttgcgaaaatgttactcaagatgggacaggagggagtcccgtctacagcggcggcttttattttagaggatgcgttaagccaaaggtcagccatggccgccgttctccacgtgcggagtcattctgaagtgccagggtttttcacagaaggaaatgacgtggcagatagccaagccaccttccaagcgtatcccttgagagaggctaaagatcttcataccgctctccatattggaccccgcgcgctatccaaagcgtgtaatatatctatgcagcaggctagggaggttgttcagacctgcccgcattgtaattcagcccctgcgttggaggccggagtaaaccctaggggtttgggacccctacagatatggcagacagactttacgcttgagcctagaatggccccccgttcctggctcgctgttactgtggataccgcctcatcagcgatagtcgtaactcagcatggccgtgtcacatcggttgctgtacaacatcattgggccacggctatcgccgttttgggaagaccaaaggccataaaaacagataatgggtcctgcttcacgtctaaatccacgcgagagtggctcgcgagatgggggatagcacacaccaccgggattccgggtaattcccagggtcaagctatggtagagcgggccaaccggctcctgaaagataggatccgtgtgcttgcggagggggacggctttatgaaaagaatccccaccagcaaacagggggaactattagccaaggcaatgtatgccctcaatcactttgagcgtggtgaaaacacgaaaacaccgatacaaaaacactggagacctaccgttcttacagaaggacccccggttaaaatacgaatagagacaggggagtgggaaaaaggatggaacgtgctggtctggggacgaggttatgccgctgtgaaaaacagggacactgataaggttatttgggtaccctctcgaaaagttaaaccggacatcacccaaaaggatgaggtgactaagaaagatgaggcgagccctctttttgcaggcatttctgactggataccctgggaagacgagcaagaaggactccaaggagaaaccgctagcaacaagcaagaaagacccggagaagacacccttgctgccaacgagagttaagcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaaccagctggggctcgaggggggatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgcctttactgagcactctttaatagtggactcttgttccaaactggaacaacactcaaccctatctcggtctattcttttgatttataagatttccatcgccatgtaaaagtgttacaattagcattaaattacttctttatatgctactattcttttggcttcgttcacggggtgggtaccgagctcgaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttggatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctggatcccgtcgacctcgagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagtctgaagccttctgcttcatgcatgtgctcgtagtcgtcagggaatcaacggtccggccatcaacccaggtgcacaccaatgtggtgaatggtcaaatggcgtttattgtatcgagctaggcacttaaatacaatatctctgcaatgcggaattcagtggttcgtccaatccgtgttagacccgtctgttgccttcctaacaaggcacgatcataccacgatcataccaccttactcccaccaatcggcatgcacggtgctttttctctccttataaggcatgttgctaactcatcgttacataagcatgttgcaagactacaagagtattgcataagactacattacgcgtcgagccgccttcaatgcccccaaaaccaatccccaggtttttaactctcccgattttccaagtaccatagcccgctgagagagcgccgcggtaatgggatcccaggaccccggggaatataagtctgagggggacgtaagcaacccttccttttgtaacagggacaacatagcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagaccggaagcttggatcaagcatggaagccgtcataaaggtgatttcgtccgcgtgtaagacctattgcgggaaaacctctccttctaagaaggaaataggggctatgttgtccctgttacaaaaggaagggttgcttatgtctccctcagacttatattccccggggtcctgggatcccattaccgc